(19) United States
(12) Patent Application Publication
(10) Pub. No.: US 2026/0218192 A1
(43) Pub. Date: Jul. 30, 2026

(54) COMPOSITIONS AND METHODS FOR TREATMENT OF MONOGENIC NEURODEVELOPMENT DISORDERS

(71) Applicant: PYC THERAPEUTICS LIMITED, Nedlands (AU)

(72) Inventors: Anja STIRNWEISS, Nedlands (AU); Dulce Vargas LANDIN, Nedlands (AU); Jessica NICHOLS, Nedlands (AU); Janya GRAINOK, Nedlands (AU); Adam MARTIN, Nedlands (AU)

(21) Appl. No.: 19/636,707

(22) Filed: Apr. 1, 2026

Related U.S. Application Data
(63) Continuation of application No. PCT/AU2024/051273, filed on Nov. 28, 2024.

(30) Foreign Application Priority Data
Nov. 29, 2023 (AU) 2023903841
Nov. 29, 2023 (AU) 2023903842
Dec. 4, 2023 (AU) 2023903916
Dec. 5, 2023 (AU) 2023903924
Dec. 5, 2023 (AU) 2023903927
Jun. 6, 2024 (AU) 2024901708
Jul. 17, 2024 (AU) 2024902223
Nov. 13, 2024 (AU) 2024903714

(51) Int. Cl. C12N 15/113; A61K 31/7088; A61P 25/28

(52) U.S. Cl. CPC C12N 15/113; A61K 31/7088; A61P 25/28; C12N 2310/321

(57) ABSTRACT

Described herein are antisense oligonucleotides (ASOs), vectors, and related compositions and methods for increasing endogenous expression of SHANK3 protein and uses thereof for conditions associated with SHANK3 haploinsufficiency such as Phelan-McDermid syndrome.

### CROSS-REFERENCE TO RELATED APPLICATIONS

The present application is a continuation application of International Patent Application No. PCT/AU2024/051273 filed on Nov. 28, 2024, which claims priority from AU 2023903841 filed on Nov. 29, 2023, AU 2023903842 filed on Nov. 29, 2023, AU 2023903916 filed on Dec. 4, 2023, AU 2023903924 filed on Dec. 5, 2023, AU 2023903927 filed on Dec. 5, 2023, AU 2024901708 filed on Jun. 6, 2024, AU 2024902223 filed on Jul. 17, 2024, and AU 2024903714 filed on Nov. 13, 2024, the entire contents each of which are incorporated herein by reference.

### SEQUENCE LISTING

The instant application contains a Sequence Listing which has been submitted electronically in XML format and is hereby incorporated by reference in its entirety. Said XML copy, created on Mar. 31, 2026, is named “047763-5033-US_SequenceListing.xml” and is approximately 22,306,874 bytes in size.

### TECHNICAL FIELD

The present disclosure generally is directed to oligonucleotides and related compositions and methods for treating conditions associated with mutations in the SHANK3 gene.

### BACKGROUND

SHANK3 is a widely expressed scaffolding protein enriched in the post-synaptic density of excitatory synapses in the brain. SHANK3 recruits and stabilizes ionotropic and metabotropic glutamate receptors (AMPA, NMDA, mGluR) to the post-synaptic density. SHANK3 gene mutations/deletions and SHANK3 haploinsufficiency underlie the rare genetic neurodevelopmental disorder, Phelan-McDermid syndrome, and have been attributed to 0.5%-1% of autism spectrum disorders (ASD), 2% of intellectual disability diagnoses, and 0.6%-2.16% of atypical schizophrenia diagnoses.

Phelan-McDermid syndrome is characterized by intellectual disability of varying degrees, neonatal hypotonia, absent to severely delayed speech development, moderate to profound developmental delay, motor regression and minor dysmorphic features. Approximately 14-70% of affected individuals develop seizures ranging from mild to severe. Other complications include kidney abnormalities, gastrointestinal issues, reduced perspiration and risk of overheating, lack of perception of pain, arachnoid cysts, or other comorbid neuropsychiatric illnesses. Children are typically diagnosed in early childhood, often due to significant delays in reaching early developmental milestones. There is currently no effective treatment for conditions caused by SHANK3 haploinsufficiency such as Phelan-McDermid syndrome. Thus, there is an ongoing need to provide effective compositions and methods for treating such conditions.

### SUMMARY

The SHANK3 gene includes 22 exons spanning 58 kb of genomic DNA on the terminal end of chromosome 22 (22q13 region) and its major protein product is a 1,607 amino acid polypeptide. There are at least six known isoforms that are temporally and spatially specific and have distinct functions at the synapse. SHANK3 contains five protein-protein interaction domains, and each isoform contains distinct combinations of these five domains.

While not wishing to be bound by theory, SHANK3 haploinsufficiency due to loss of function gene mutation(s), including nonsense, missense and frameshift mutations, as well as partial or whole gene deletions, results in insufficient protein production.

The present disclosure provides antisense oligonucleotides (ASO), antisense RNA (AR) expression vectors, and related compositions and methods to increase SHANK3 protein levels by modulating the stability of SHANK3 mRNA to increase the level of SHANK3 mRNAs encoding functional SHANK3 isoforms. Also disclosed are methods for treating conditions associated with SHANK3 haploinsufficiency.

Accordingly, in one aspect provided herein is an antisense oligonucleotide that binds within a targeted portion of the 3′ UTR of a SHANK3 mRNA (SEQ ID NO:1);

whereby binding of the antisense oligonucleotide within the targeted portion in a mammalian cell results in an increased level of SHANK3 protein in the mammalian cell. In some examples the targeted portion comprises the sequence according to SEQ ID NO:1079. In some examples the targeted portion consists of the sequence according to SEQ ID NO:1079.

In a related aspect provided herein is vector for expression, in a mammalian neuron, of an antisense RNA (AR) that binds within a targeted portion of the 3′ UTR of a SHANK3 mRNA;

whereby binding of the AR within the targeted portion in a mammalian cell results in an increased level of SHANK3 protein in the mammalian cell.

In some examples the sequence of the SHANK3 mRNA 3′ UTR comprises the sequence corresponding to SEQ ID NO:1. In some examples the targeted portion comprises the sequence according to SEQ ID NO:1079. In some examples the targeted portion consists of the sequence according to SEQ ID NO:1079.

In some examples the nucleotide sequence of the ASO or AR is at least 60%, 65%, 70%, 75%, 80%, 85%, 90%, 95%, or 100% complementary to the nucleotide sequence of the targeted portion over the length of the ASO or the AR.

In some examples the mammalian cell in which a SHANK3 level is increased is a neuron.

In some examples the vector includes a neuron-selective promoter for driving expression of the AR in the mammalian neuron. In some examples the neuron-selective promoter is selective for expression in a neuron type selected from the list consisting of cortical, striatal, cerebellar and hippocampal excitatory or inhibitory neurons, including, but not limited to cortical glutamatergic neurons, cortical GABAergic neurons, hippocampal glutamatergic neurons, and striatal inhibitory neurons. In some examples the vector includes an inducible promoter. In some examples the vector is a non-viral vector. In some examples a non-viral vector further comprises a transfection agent. In other examples the vector is a viral vector. In some examples, where the vector is a viral vector, the viral vector is a recombinant virus selected from the group consisting of: adeno-associated virus (AAV), adenovirus, lentivirus, and anellovirus.

In some examples the nucleotide sequence of the ASO or AR comprises up to two base mismatches to the targeted portion.

In some examples the nucleotide sequence of the ASO or AR corresponds to any one of SEQ ID NOs:1080-1093.

In some examples the nucleotide sequence of the ASO corresponds to any one of SEQ ID NOs:1080-1093 or 1096-1133. In some examples the nucleotide sequence of the ASO corresponds to any one of SEQ ID NOs:1080-1093. In some examples the nucleotide sequence of the ASO corresponds to any one of SEQ ID NOs:1084, 1087, or 1091-1093. In some examples the nucleotide sequence of the ASO corresponds to SEQ ID NO:1084. In some examples the nucleotide sequence of the ASO corresponds to SEQ ID NO:1087. In some examples the nucleotide sequence of the ASO corresponds to SEQ ID NO:1091. In some examples the nucleotide sequence of the ASO corresponds to SEQ ID NO:1092. In some examples the nucleotide sequence of the ASO corresponds to SEQ ID NO:1093.

In some examples the nucleotide sequence of the ASO or AR corresponds to any one of SEQ ID NOs:1096-1133. In some examples the nucleotide sequence of the ASO or AR corresponds to any one of SEQ ID NOs:1096, 1109, 1119, and 1128. In some examples the nucleotide sequence of the ASO corresponds to SEQ ID NO:1096. In some examples the nucleotide sequence of the ASO corresponds to SEQ ID NO:1109. In some examples the nucleotide sequence of the ASO corresponds to SEQ ID NO:1119. In some examples the nucleotide sequence of the ASO corresponds to SEQ ID NO:1128.

In some examples any of the foregoing ASOs include a backbone modification. In some examples the backbone modification includes a phosphorothioate linkage or a phosphorodiamidate linkage. In other examples the ASO includes a phosphorodiamidate morpholino, an arabinonucleic acid, a locked nucleic acid, a peptide nucleic acid, a 2′-O-modification such as a 2′-O-methyl, a 2′-Fluoro, or a 2′-O-methoxyethyl moiety, or an abasic spacer subunit. In some examples the abasic spacer subunit comprises the structure according to Formula I.

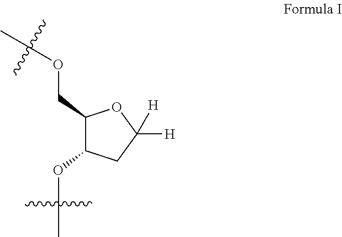 [SMILES: *OC[C@H]1OCC[C@@H]1O*]

In some examples the nucleotide sequence of the ASO comprises one or more abasic spacer subunits and/or locked nucleic acids. In some examples the nucleotide sequence of the ASO comprises one or more abasic spacer subunits and one or more locked nucleic acids. In some examples, where the ASO comprises at least one abasic spacer subunit, at least one locked nucleic acid, or both, the nucleotide sequence of the ASO corresponds to any one of SEQ ID NOs:1096-1133. In some examples the nucleotide sequence of the ASO or AR targeting the 3′ UTR corresponds to any one of SEQ ID NOs:3-1133, SEQ ID NOs:1134-4583, and SEQ ID NOs:16254-18750.

In some examples the ASO includes at least one modified sugar moiety. In other examples each sugar moiety in the ASO is a modified sugar moiety. In some examples the ASO includes a 2′-O-methoxyethyl moiety. In other examples each nucleotide of the ASO includes a 2′-O-methoxyethyl moiety.

In some examples of any of the foregoing ASOs or vectors, the nucleotide sequence of the ASO or AR is 10 to 50 nucleotides, 15 to 40 nucleotides, 17 to 30 nucleotides, 15 to 30 nucleotides, 18 to 40 nucleotides, 17 to 25 nucleotides, 20 to 35 nucleotides, 15 to 30 nucleotides, 20 to 30 nucleotides, 22 to 30 nucleotides, 24 to 30 nucleotides, 25 to 30 nucleotides, or 26 to 30 nucleotides in length. In some examples, where the ASO is 17 to 30 nucleotides in length, the ASO includes one or more phosphorodiamidate morpholino moieties. In other examples, where the ASO is 15 to 30 nucleotides in length, the ASO includes one or more phosphorodiamidate morpholino moieties.

In some examples any of the foregoing ASOs are linked to a functional moiety. In some examples the functional moiety includes a delivery moiety. In some examples the delivery moiety is selected from the group consisting of: lipids, polyethers, peptides, carbohydrates, receptor binding peptide (RBP), and antibodies. In some examples, wherein the ASO includes a delivery moiety, the delivery moiety includes a cell-penetrating peptide (CPP). In some examples the CPP comprises the amino acid sequence corresponding to SEQ ID NO:2. In some examples the delivery moiety includes a N-acetylgalactosamine (GalNAc) or glycan moiety. In some examples the delivery moiety includes a fatty acid or lipid moiety. In some embodiments the fatty acid chain length is about C8 to C20. In other examples the functional moiety includes a stabilising moiety. In some examples the functional moiety is covalently linked to the ASO. In other examples the functional moiety is non-covalently linked to the ASO. In some examples the functional moiety is linked to the 5′ end of the ASO. In other examples the functional moiety is linked to the 3′ end of the ASO. In some examples any of the foregoing ASOs also include a delivery nanocarrier, wherein the nanocarrier is complexed with the ASO. In some examples the delivery nanocarrier is selected from the group consisting of: lipoplexes, liposomes, exosomes, inorganic nanoparticles, and DNA nanostructures. In some examples the delivery nanocarrier includes a lipid nanoparticle (LNP) encapsulating the ASO.

In a related aspect provided herein is a pharmaceutical composition that includes any of the foregoing ASOs, vectors, or compositions and a pharmaceutically acceptable excipient. In some preferred embodiments the pharmaceutical compositions comprises an ASO as disclosed herein. In some embodiments the pharmaceutical composition is provided as a gymnotic formulation of the ASO (i.e., a formulation devoid of agents to facilitate transfection of the ASO).

In a further related aspect provided herein is a method for preventing or treating a condition associated with SHANK3 haploinsufficiency, the method comprising administering to a subject in need thereof a therapeutically effective amount of the foregoing pharmaceutical composition. In some examples the condition to be treated is Phelan-McDermid syndrome, an autism spectrum disorder, schizophrenia, or an intellectual disability. In some examples the condition to be treated is Phelan-McDermid syndrome. In some examples the subject to be treated is a human subject.

In a further aspect provided herein is the use of any of the foregoing antisense oligonucleotides or vectors in the manufacture of a medicament for prevention or treatment of a condition associated with SHANK3 haploinsufficiency.

In some examples of the foregoing methods of treatment or uses, the level of SHANK3 protein in at least a plurality of cells in the subject is increased about 1.1 to about 5 fold in cells (e.g., neurons) in vitro or ex vivo, e.g., 1.2 fold, 1.3 fold, 1.5 fold, 1.7 fold, 2 fold, 2.2 fold, 2.5 fold, 2.7 fold, 3 fold, 3.3 fold, 3.5 fold, 4 fold, 4.3 fold, 4.5 fold, 4.7 fold, or another increase in SHANK3 protein levels from about 1.1 fold to about 5 fold in cells in a subject compared to the level in the absence of the pharmaceutical composition.

In some examples of the foregoing methods of treatment or uses, the ratio of the levels of SHANK3 protein essential isoforms relative to each other following the treatment are unchanged.

In yet another aspect provided herein is a genetically modified cell comprising any of the foregoing ASOs or vectors. In some examples the genetically modified cell is a mammalian cell. In some examples the genetically modified mammalian cell is a human cell. In some examples the genetically modified mammalian cell is a genetically modified neuron or neural progenitor. In some examples, the genetically modified neuron is selected from the group consisting of: cortical, striatal, cerebellar and hippocampal excitatory or inhibitory neurons, including, but not limited to cortical glutamatergic neurons, cortical GABAergic neurons, hippocampal glutamatergic neurons, and striatal inhibitory neurons. In some examples the genetically modified neuron is a cortical glutamatergic neuron obtained by differentiation of an hiPSC line, wherein the hiPSC line is derived from a subject suffering from Phelan-McDermid syndrome or a subject not suffering from Phelan-McDermid syndrome. In some embodiments the genetically modified mammalian cell is not a human embryonic cell. In some examples the genetically modified mammalian cell is from a cell line. In some examples the cell line is a human induced pluripotent stem cell (hiPSC) line or a cell line derived from neurons.

In other examples the level of SHANK3 can be increased by modulating splicing (including alternative splicing) of SHANK3 pre-mRNA transcripts. The term alternative splicing refers to a process whereby exons, or part of an exon of a gene, or introns or part of an intron may be included within or excluded from the final mRNA transcript. Mature noncanonical mRNA transcripts can be non-productive due to a frame shift that may induce the nonsense mediated decay pathway. In other cases, translation of the noncanonical mRNA can produce a truncated but non-functional protein. Alternative splicing of the SHANK3 pre-RNA transcripts can downregulate overall SHANK3 mRNA and protein expression.

Introns are removed by a large RNA-protein complex termed the spliceosome, which orchestrates complex interactions between primary transcripts, small nuclear RNAs (snRNAs) and a large number of proteins. Spliceosomes assemble on each intron in an ordered manner, starting with recognition of the 5′ splice site (5′ss) by U1 snRNA or the 3′ splice site (3′ ss) by the U2 pathway, which involves binding of the U2 auxiliary factor (U2AF) to the 3′ ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ ss and stabilizes U2AF65 binding. In addition to the BPS/PPT unit and 3′ ss/5′ ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude.

In some examples the present disclosure provides antisense oligonucleotides (ASO), antisense RNA (AR) expression vectors, and related compositions and methods to increase SHANK3 protein levels. While not wishing to be bound by theory, it is believed that in some examples ASOs effect an increase in SHANK3 protein levels by modulating splicing of SHANK3 mRNA to increase the level of canonical SHANK3 mRNA encoding full length, functional SHANK3. More specifically, it is believed that the ASOs modulate splicing to decrease aberrant intron retention, particularly any one of introns 6, 7, 16, 17, 18, or 20. Also disclosed are methods for treating conditions associated with SHANK3 haploinsufficiency.

Accordingly, in one aspect provided herein is an antisense oligonucleotide that binds within a targeted portion of a SHANK3 pre-mRNA, whereby the level of SHANK3 mRNA encoding full length, functional SHANK3 is increased, wherein the sequence of the targeted portion corresponds to SEQ ID NO:1 or SEQ ID NO:18751.

In a related aspect provided herein is a vector for expression, in a mammalian neuron, of an antisense RNA (AR) that binds within a targeted portion of a SHANK3, whereby the level of SHANK3 mRNA encoding full length, functional SHANK3 is increased, wherein the sequence of the targeted portion corresponds to SEQ ID NO:1. In some examples the vector includes a neuron-selective promoter for driving expression of the antisense RNA in the mammalian neuron. In some examples the neuron-selective promoter is selective for expression in a neuron type selected from the list consisting of: cortical excitatory neurons, cortical inhibitory neurons, hippocampal excitatory neurons, hippocampal inhibitory neurons, striatal inhibitory neurons, striatal excitatory neurons, cerebellar excitatory neurons, cerebellar inhibitory neurons, and GABAergic neurons. In some examples the vector includes an inducible promoter. In some examples the vector is a non-viral vector. In some examples a non-viral vector further comprises a transfection agent. In other examples the vector is a viral vector. In some examples, where the vector is a viral vector, the viral vector is a recombinant virus selected from the group consisting of: adeno-associated virus (AAV), adenovirus, lentivirus, and anellovirus.

In some examples of any of the foregoing ASOs or vectors, binding of the antisense oligonucleotide or AR to the SHANK3 pre-mRNA modulates splicing of a SHANK3 mRNA derived therefrom In some examples of any of the foregoing methods, ASOs, vectors, or compositions, the modulation of splicing reduces a level of aberrant retention of an intron. In some examples the sequence of an antisense oligonucleotide or AR disclosed herein includes up to two base mismatches to the targeted portion.

In some examples the nucleotide sequence of the ASO or AR is at least 60%, 65%, 70%, 75%, 80%, 85%, 90%, 95%, or 100% complementary to the nucleotide sequence of the targeted portion over the length of the ASO or the AR. In some examples the nucleotide sequence of the ASO or AR that binds to a targeted portion of the SHANK3 pre-mRNA corresponds to any one of SEQ ID NOs:4584-16253.

In some examples any of the foregoing ASOs include a backbone modification. In some examples the backbone modification includes a phosphorothioate linkage or a phosphorodiamidate linkage. In other examples the ASO includes a phosphorodiamidate morpholino, an arabinonucleic acid, a locked nucleic acid, a peptide nucleic acid, or a 2′-O-modification such as a 2′-O-methyl, a 2′-Fluoro, or a 2′-O-methoxyethyl moiety. In some examples the ASO includes at least one modified sugar moiety. In other examples each sugar moiety in the ASO is a modified sugar moiety. In some examples the ASO includes a 2′-O-methoxyethyl moiety. In other examples each nucleotide of the ASO includes a 2′-O-methoxyethyl moiety.

In some examples of any of the foregoing ASOs or vectors, the nucleotide sequence of the ASO or AR is 10 to 50 nucleotides, 15 to 40 nucleotides, 18 to 40 nucleotides, 17 to 25 nucleotides, 20 to 35 nucleotides, 20 to 30 nucleotides, 22 to 30 nucleotides, 24 to 30 nucleotides, 25 to 30 nucleotides, or 26 to 30 nucleotides in length. In some examples. In some examples of any of the foregoing ASOs or vectors, the nucleotide sequence of the ASO or AR is 17 to 30 nucleotides in length.

In some examples any of the foregoing ASOs also includes a linked functional moiety. In some examples the functional moiety includes a delivery moiety. In some examples the delivery moiety is selected from the group consisting of lipids, peptides, carbohydrates, and antibodies. In some examples, wherein the ASO includes a delivery moiety, the delivery moiety includes a cell-penetrating peptide (CPP). In some examples the delivery moiety includes a N-acetylgalactosamine (GalNAc) or glycan moiety. In some examples the delivery moiety includes a fatty acid or lipid moiety. In some embodiments the fatty acid chain length is about C8 to C20. In other examples the functional moiety includes a stabilising moiety. In some examples the functional moiety is covalently linked to the ASO. In other examples the functional moiety is non-covalently linked to the ASO. In some examples the functional moiety is linked to the 5′ end of the ASO. In other examples the functional moiety is linked to the 3′ end of the ASO. In some embodiments of any of the foregoing methods, the level of SHANK3 protein in at least a plurality of mammalian cells is increased about 1.1 to about 5 fold in cells (e.g., neurons) in vitro or ex vivo, e.g., 1.2 fold, 1.3 fold, 1.5 fold, 1.7 fold, 2 fold, 2.2 fold, 2.5 fold, 2.7 fold, 3 fold, 3.3 fold, 3.5 fold, 4 fold, 4.3 fold, 4.5 fold, 4.7 fold, or another increase in SHANK3 protein levels from about 1.1 fold to about 5 fold in cells in a subject or in vitro or in vivo, compared to the level in a plurality of mammalian cells in the absence of the oligonucleotide or the antisense RNA.

In a related aspect provided herein is a pharmaceutical composition that includes any of the foregoing ASOs or vectors and a pharmaceutically acceptable excipient.

In a further related aspect provided herein is a method for preventing or treating a condition associated with SHANK3 haploinsufficiency, the method comprising administering to a subject in need thereof a therapeutically effective amount of the foregoing pharmaceutical composition. In some examples the condition to be treated is Phelan-McDermid syndrome, an autism spectrum disorder, schizophrenia, or an intellectual disability. In some examples the condition to be treated is Phelan-McDermid syndrome. In some examples the subject to be treated is a human subject.

In a further aspect provided herein is the use of any of the foregoing antisense oligonucleotides, vectors, or compositions in the manufacture of a medicament for prevention or treatment of a condition associated with SHANK3 haploinsufficiency.

In some examples of the foregoing methods of treatment or uses, the level of SHANK3 protein in a least a plurality of cells in the subject is increased about 1.1 to about 5 fold in cells (e.g., neurons) in vitro or ex vivo, e.g., 1.2 fold, 1.3 fold, 1.5 fold, 1.7 fold, 2 fold, 2.2 fold, 2.5 fold, 2.7 fold, 3 fold, 3.3 fold, 3.5 fold, 4 fold, 4.3 fold, 4.5 fold, 4.7 fold, or another increase in SHANK3 protein levels from about 1.1 fold to about 5 fold in cells in a subject or in vitro or in vivo. compared to the level in the absence of the pharmaceutical composition.

In yet another aspect provided herein is a genetically modified cell comprising any of the foregoing ASOs or vectors. In some examples the genetically modified cell is a mammalian cell. In some examples the genetically modified mammalian cell is a human cell. In some examples the genetically modified mammalian cell is a neuron or a neural progenitor. In some examples, the genetically modified mammalian cell is a neuron selected from the group consisting of: cortical glutamatergic neurons, cortical GABAergic neurons, hippocampal glutamatergic neurons, and striatal inhibitory neurons. In some examples the genetically modified mammalian cell is from a cell line. In some examples the cell line is a hiPSC cell line or a cell line derived from neurons.

### BRIEF DESCRIPTION OF THE ACCOMPANYING DRAWINGS

FIG. 1—Illustration of SHANK3 transcript and ASO target region. (A) SHANK3 transcript model (gene id ENSG00000251322.11 from Gencode v45). Exons and introns are indicated in black color and 5′ and 3′ UTRs indicated in lighter color. ASOs were designed to target the 3′ UTRs of SHANK3 transcript to mediate upregulation of SHANK3 gene expression. B) seed sequence of hsa-mir-504-5p, a target sequence for the miRNA hsa-mir-504-5p. (C) Negative SHANK3 gene expression regulation partly induced by the binding of hsa-mir-504-5p to the 3′ UTR of SHANK3 mRNA at its target site sequence, resulting in a reduction of SHANK3 protein. (D) illustration of a model in which a steric-blocking ASO binds directly to the 3′ UTR of SHANK3 transcripts inhibiting the binding of hsa-mir-504-5p and its induction of RNA degradation leading to a net increase in SHANK3 transcript levels and SHANK3 protein production.

FIG. 2—Human whole brain SHANK3 mRNA and hsa-mir-504-5p expression during human neurodevelopment. SHANK3 mRNA expression was measured using poly-A enriched bulk single-ended RNA-seq data from samples across all brain regions at 9 prenatal and 18 postnatal time points. (PCW, post-conception week; M, months; Y, years; cpm, counts per million)

FIG. 3—PPMO screen in SH-SY5Y cells to upregulate SHANK3 protein expression. SHANK3 protein expression was assessed in PPMO treated SH-SY5Y cells using an ELISA assay. A non-targeting control (NTC), predicted not to hybridize to human transcripts, was included as a negative control. Bar graph represents mean±S.D. n=2 independent experiments with 3 technical replicates per experiment. UT=untreated cells. NTC=non-targeting control. PPMOs with SEQ ID NOs: 160 and 687 were selected for further evaluation in iPSC-derived neurons.

FIG. 4—PPMO effect on SHANK3 protein expression in iPSC-derived glutamatergic neurons. iPSC-derived glutamatergic neurons derived from healthy controls (non-PMS controls) were incubated with a peptide-conjugated PMO (SEQ ID NOs: 160 and 687) in triplicate and a non-targeting control (NTC) for 7 days. A non-targeting control, predicted not to hybridize to human transcripts, was included as a negative control. Bar graph represents mean±S.D. of SHANK3 protein normalized to total protein amount loaded relative that of untreated cells. n=1 biological replicate per cell line. UT=untreated cells. NTC=non-targeting control.

FIG. 5—SHANK3 protein analysis in PPMO-treated iPSC-glutamatergic neurons derived from Phelan-McDermid Syndrome (PMS). iPSCs derived from in a PMS patient harboring a heterozygous large deletion in the SHANK3 gene, were differentiated into glutamatergic neurons used to test the efficacy of a PPMO targeted to the SHANK3 transcript. The PPMO has an oligonucleotide sequence corresponding to “SEQ ID NO: 1847” (originally disclosed in WO 2023/235915) linked to a CPP (SEQ ID NO:2 originally disclosed as “SEQ ID NO:12694” in WO 2023/235915) and was incubated with iPSC-derived glutamatergic neurons in triplicate for 7 days. A non-targeting control, predicted not to hybridize to human transcripts, was included as a negative control. Bar graph represents mean±S.D. of SHANK3 protein normalized to total protein amount loaded relative that of untreated cells. n=1 biological replicate. UT=untreated cells. Graph was generated using GraphPad Prism Ver 10.2.3 software. *p<0.05, **p<0.005, ***p<0.001. One-Way ANOVA.

FIG. 6—SHANK3 protein isoform analysis in ASO-treated hiPSC (non-affected subject)-derived glutamatergic neurons. iPSC-derived glutamatergic neurons were incubated with an ASO (SEQ ID NO: 1193 disclosed in WO 2023/235915) in triplicate for 7 days. Bar graph represents mean±S.D. of the SHANK3 protein isoform, normalized to the respective SHANK3 isoform relative to that of untreated cells. n=1 biological replicate per cell line. UT=untreated cells. Levels of each of the major isoforms evaluated were increased, and the relative ratio of expression levels between the essential isoforms appears unchanged.

FIG. 7—Illustration of SHANK3 transcript and ASO design within the target region of the 3′ UTR of SHANK3 transcript. (A) SHANK3 transcript model (gene id ENSG00000251322.11 from Gencode v45) is depicted, with exons and introns shown in dark gray and the 5′ and 3′ UTRs in a lighter shade. (B) A segment of the 3′ UTR sequence corresponding to SEQ ID NO:1079. The miR-504 seed sequence is highlighted in bold. (C) A black line indicates the target sequences of ASOs, designed to upregulate SHANK3 gene expression. This is achieved by altering the secondary structure of mRNA or steric blocking the binding of regulators that reduce the efficacy of SHANK3 protein translation. The numbers correspond to SEQ ID numbers of ASOs as disclosed herein (see also Tables 1-3 of the Appendix); locked nucleic acid (LNA)-modified sequences are marked with a (*) after their SEQ ID number.

FIG. 8—Effect of 2′MOEs on SHANK3 protein levels in human iPSC-derived glutamatergic neurons. The bar plots represent the mean±SD of SHANK3 protein normalized to the total protein amount loaded, relative to the normalized SHANK3 protein of untreated cells (y-axis). Data is derived from three technical replicates from one biological replicate. Bar plots in white represent untreated cell controls (UT) and to non-targeting control (NTC) at 1 μM; grey bars represent ASO-treated cells at 0.5 μM; and black bars represent ASO-treated cells at 1 μM. ASOs are split by length. The dotted line represents the baseline of untreated cells at 1. UT refers to untreated cells and NTC indicates the non-targeting control. Graph was generated using GraphPad Prism Version 10.2.3 software.

FIG. 9—Effect of selected MOEs on SHANK3 protein levels in human iPSC-derived neurons. The bar plots represent the mean±SD of SHANK3 protein normalized to the total protein amount loaded, relative to the normalized SHANK3 protein of untreated cells (y-axis). Data is derived from three technical replicates from one biological replicate. Bar plots in colour white correspond to untreated cell controls (UT) and to non-targeting control (NTC) at highest concentration (30 μM); striped pattern to cells treated with a 7.5 μM dose; grey colour to cells treated with a 15 μM dose; and black colour to cells treated with a 30 μM dose. The bar graphs are grouped by ASO length used in the corresponding experiments. The dotted line represent the baseline of untreated cells at 1.0-fold change. UT stands for untreated cells and NTC for non-targeting control. Graph was generated using GraphPad Prism Version 10.2.3 software.

FIG. 10—Effect of selected LNA modified 2′MOEs on SHANK3 protein levels in human iPSC-derived neurons. The bar plots represent the mean±SD of SHANK3 protein normalized to the total protein amount loaded, relative to the normalized SHANK3 protein of untreated cells (y-axis). Data is derived from three technical replicates from one biological replicate. Bar plots in colour white correspond to untreated cell controls (UT) and to non-targeting control (NTC) at highest concentration (30 μM); striped pattern to cells treated with a 7.5 μM dose; grey colour to cells treated with a 15 μM dose; and black colour to cells treated with 30 μM dose. The dotted line represents the baseline of untreated cells at 1.0-fold change. Panel (A) shows the SHANK3 protein changes after the treatment with 18-mer ASOs with sequences corresponding to SEQ ID NOs: 1091, 1093, and 1092; while panel (B) shows the ASO derivates from a 15-mer, a 17-mer and a 20-mer with sequences corresponding to SEQ ID NOs: 1084, 1094, and 1087, respectively. UT stands for untreated cells and NTC for non-targeting control. Graphs were generated using GraphPad Prism Version 10.2.3 software.

FIG. 11—Effect of selected MOEs on SHANK3 protein levels in human iPSC-derived neurons from Phelan-McDermid Syndrome patients. The bar plots represent the mean±SD of SHANK3 protein normalized to the total protein amount loaded, relative to the normalized SHANK3 protein of untreated cells (y-axis). Data is derived from three technical replicates from one biological replicate. Panel (A) shows changes in SHANK3 protein levels following treatment with ASOs corresponding to SEQ ID NOs: 1094, 1087, 1091, 1093, and 1092; in which the white bar indicates untreated controls (UT); bars with stripes represent cells treated at 7.5 μM; bars with a dotted pattern indicate cells treated at 15 μM; and bars in solid gray represent cells treated at 30 μM; and black represent cells treated at 45 μM. Panel (B) shows changes in SHANK3 protein levels after treatment with SEQ ID NOs: 1122, 1093, 1091, 1126, 1128, and 1092. White bar represent untreated controls (UT), striped bar for 7.5 μM, gray bar for 15 μM dose, and black bar for 30 μM. In both panels, dotted lines represent the baseline of untreated cells at 1.0-fold change. UT denotes untreated cells. Graphs were generated using GraphPad Prism Version 10.2.3 software. * p. adj. <0.05, ** p. adj. <0.01, *** p. adj. <0.005, **** p. adj. <0.001. One-Way ANOVA.

FIG. 12—Functional assessment of gymnotic ASO treatment in Phelan-McDermid patient iPSC-derived glutamatergic neurons. The bar plots represent the mean±SD of the signal intensity of spontaneous calcium oscillations (y-axis). Data is derived from three technical replicates from one biological replicate. Panel (A) shows the baseline signal intensity of spontaneous calcium oscillations in two untreated non-PMS cell lines (SHANK3-non-deficient) shown in white and grey, two untreated PMS patient cell lines (SHANK3-deficient) shown in black. The left side (including PMS patient #1) of the plot corresponds to a 20,000-30,000 cells seeding; while the right side (including PMS patient #2) to a 10,000 cells seeding. The dotted line represents the baseline level of spontaneous calcium oscillations signal in untreated SHANK3-non-deficient commercial cells at 1.0-fold change. Panel (B) shows the functional assessment in PMS patient cells 21 days after a single-dose ASO treatment. White bars represent untreated cell control (UT) and non-targeting control (NTC); grey bars represent cells treated with a 15 μM ASO; black bar represent cells treated with a 30 μM concentration. The dotted line represents the untreated baseline at 1.0-fold change. Panel (C) shows the functional assessment in PMS patient cells 21 days after a double-dose ASO treatment, White bar represent control group including untreated cell control (UT), non-targeting control (NTC), negative assay control (BAPTA) and positive assay control (Ionomycin). Black bar represent cells treated with ASO at 15 μM. The dotted line represents the baseline of untreated cells at 1.0-fold change. UT stands for untreated cells and NTC to non-targeting control. BAPTA is used as an assay control to chelate free calcium ions, thereby suppressing calcium-dependent signaling. Ionomycin acts as a calcium ionophore, increasing intracellular calcium levels by facilitating calcium entry into cells. Graphs were generated using GraphPad Prism Version 10.2.3 software.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The present disclosure is performed without undue experimentation using, unless otherwise indicated, conventional techniques of molecular biology, microbiology, virology, recombinant DNA technology, peptide synthesis in solution, solid phase peptide synthesis, and immunology. Such techniques are described and explained throughout the literature in sources such as Perbal 1984, Sambrook et al., 2001, Brown (editor) 1991, Glover and Hames (editors) 1995 and 1996, Ausubel et al. including all updates until present, Coligan et al. (editors) (including all updates until present), Maniatis et al. 1982, Gait (editor) 1984, Hames and Higgins (editors) 1984, Freshney (editor) 1986.

The term “and/or”, e.g., “X and/or Y” shall be understood to mean either “X and Y” or “X or Y” and shall be taken to provide explicit support for both meanings or for either meaning.

The term “about”, unless stated to the contrary, refers to ±20%, more preferably ±10%, of the designated value. For the avoidance of doubt, the term “about” followed by a designated value is to be interpreted as also encompassing the exact designated value itself (for example, “about 10” also encompasses 10 exactly).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “antisense oligonucleotide”, “antisense oligomer” or “ASO,” as used herein, encompasses oligonucleotides and any other oligomeric molecule that comprises nucleobases capable of hybridizing to a complementary sequence on a target RNA transcript, including, but not limited to, those that do not comprise a sugar moiety, such as in the case of a peptide nucleic acid (PNA). Preferably, the ASO is an ASO that is resistant to nuclease cleavage or degradation.

The phrase “binds to a targeted portion” or “binds within a targeted portion,” in reference to an ASO or AR, as used herein, refers to specific hybridization between the ASO or AR nucleotide sequence and a target nucleotide sequence that is complementary within the ranges set forth herein. In some examples the targeted portion comprises the sequence according to SEQ ID NO:1079. In some examples the targeted portion consists of the sequence according to SEQ ID NO:1079. In some examples, specific hybridization occurs where, under ex vivo conditions, the hybridization occurs under high stringency conditions. By “high stringency conditions” is meant that the ASO or AR, under such ex vivo conditions, hybridize to a target sequence in an amount that is detectably stronger than non-specific hybridization. High stringency conditions, then, are conditions that distinguish a polynucleotide with an exact complementary sequence, or one containing only a few scattered mismatches from a random sequence that happened to have a few small regions (e.g., 1-5 bases) that matched the probe. Such small regions of complementarity are more easily melted than a full-length complement of 12-17 or more bases, and moderate stringency hybridization makes them easily distinguishable. In one example, high stringency conditions include, for example, low salt and/or high temperature conditions, such as provided by about 0.02-0.1 M NaCl or the equivalent, at temperatures of about 50-70° C. The skilled person will appreciate that under in vivo conditions, the specificity of hybridization between an ASO or an AR and its target sequence is defined in terms of the level of complementarity between the ASO or an AR and the target sequence to which it hybridizes within a cell.

The phrase “antisense oligonucleotide or AR comprises a mismatch to the targeted region” as used herein refers to the fact that the antisense oligonucleotide or AR sequence differs from the reverse complement of the target sequence with which it is to hybridize at the referred-to (mismatched) position. For example, for a target sequence:

5′-AGUGGAUUGCUAGU-3′, a perfectly complementary ASO sequence would include 5′-ACUAGCAAUCCACU-3′, where the ASO 5′-ACUAGCACUCCACU-3′ includes a “base mismatch” with respect to the target sequence at the underlined/bold position.

The term “gymnotic” in reference to an ASO or ASO delivery as used herein refers to delivery of an ASO into cells in the absence of a transfection agent.

The term “peptide” is intended to include compounds composed of amino acid residues linked by amide bonds. A peptide may be natural or unnatural, ribosome translated or synthetically derived. Typically, a peptide will consist of between 2 and 200 amino acids. For example, the peptide may have a length in the range of 10 to 20 amino acids or 10 to 30 amino acids or 10 to 40 amino acids or 10 to 50 amino acids or 10 to 60 amino acids or 10 to 70 amino acids or 10 to 80 amino acids or 10 to 90 amino acids or 10 to 100 amino acids, including any length within said range(s). The peptide may comprise or consist of fewer than about 150 amino acids or fewer than about 125 amino acids or fewer than about 100 amino acids or fewer than about 90 amino acids or fewer than about 80 amino acids or fewer than about 70 amino acids or fewer than about 60 amino acids or fewer than about 50 amino acids.

Peptides, as referred to herein, include “inverso” peptides in which all L-amino acids are substituted with the corresponding D-amino acids, “retro-inverso” peptides in which the sequence of amino acids is reversed and all L-amino acids are replaced with D-amino acids.

Peptides may comprise amino acids in both L- and/or D-form. For example, both L- and D-forms may be used for different amino acids within the same peptide sequence. In some examples the amino acids within the peptide sequence are in L-form, such as natural amino acids. In some examples the amino acids within the peptide sequence are a combination of L- and D-form. Further, peptides may comprise unusual, but naturally occurring, amino acids including, but not limited to, hydroxyproline (Hyp), beta-alanine, citrulline (Cit), omithine (Orn), norleucine (Nle), 3-nitrotyrosine, nitroarginine, pyroglutamic acid (Pyr). Peptides may also incorporate unnatural amino acids including, but not limited to, homo amino acids, N-methyl amino acids, alpha-methyl amino acids, beta (homo) amino acids, gamma amino acids, and N-substituted glycines. Peptides may be linear peptides or cyclic peptides.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical bond or a disulfide bond. Examples of non-covalent bonds include hydrogen bonds, ionic bonds, Van der Waals forces, and hydrophobic interactions.

Percentage amino acid sequence identity with respect to a given amino acid sequence is defined as the percentage of amino acid residues in a candidate sequence that are identical to the amino acid residues in the reference sequence, after aligning the sequences and introducing gaps, if necessary, to achieve the maximum percent sequence identity, and not considering any conservative substitutions as part of the sequence identity. Amino acid sequence identity may be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized, which include Gap Open: 10.0 and Gap Extend 0.5. The default matrix “Blosum62” is utilized for amino acid sequences and the default matrix. Percent (%) or percentage “nucleic acid sequence identity” with respect to the nucleotide sequences disclosed herein is defined as the percentage of nucleotides in a candidate sequence that are identical with the nucleotides in the reference sequence, after aligning the sequences and introducing gaps, if necessary, to achieve the maximum percent sequence identity. Alignment for purposes of determining percent nucleic acid sequence identity can be achieved in various ways that are known in the art, for instance, using publicly available computer software such as BLAST or ALIGN. The skilled person can readily determine appropriate parameters for measuring alignment, including any algorithms needed to achieve maximal alignment over the full length of the sequences being compared.

The term “cell penetrating peptide” (CPP) refers to a peptide that is capable of crossing a cellular membrane. In one example, a CPP is capable of translocating across a mammalian cell membrane and entering into a cell. In another example, a CPP may direct a conjugate to a desired subcellular compartment. Thus, a CPP may direct or facilitate penetration of a molecule of interest across a phospholipid, mitochondrial, endosomal, lysosomal, vesicular, or nuclear membrane. A CPP may be translocated across the membrane with its amino acid sequence complete and intact, or alternatively partially degraded.

A CPP may direct a molecule of interest, such as an ASO disclosed herein, from outside a cell through the plasma membrane, and into the cytoplasm or a desired subcellular compartment. Alternatively, or in addition, a CPP may direct a molecule of interest across the blood-brain, trans-mucosal, hematoretinal, skin, gastrointestinal and/or pulmonary barriers.

The term “peptide ligand” or “receptor binding peptide” refers to a peptide that is capable of binding to a membrane surface receptor to enable transport of the peptide across a cellular membrane. In one example a peptide ligand may enable transport across the cellular membrane via the natural endocytosis of the targeted receptor. In another example the peptide ligand may utilise a complementary mechanism of transport across the cellular membrane including utilising a conjugated CPP. In one example, a peptide ligand is capable of translocating across a mammalian cell membrane and to enter a cell. In another example, a peptide ligand may direct a conjugate to a desired subcellular compartment. Thus, a peptide ligand may direct or facilitate cellular uptake of a molecule of interest across a phospholipid, mitochondrial, endosomal, lysosomal, vesicular, or nuclear membrane. A peptide ligand may be translocated across the membrane with its amino acid sequence complete and intact, or alternatively partially degraded.

A peptide ligand via its binding to a target receptor may direct a molecule of interest, such as an ASO disclosed herein, from outside a cell through the plasma membrane, and into the cytoplasm or a desired subcellular compartment. Alternatively, or in addition, a peptide ligand via its binding to a target receptor may direct a molecule of interest across a relevant biological barrier, e.g., the blood-brain, trans-mucosal, hematoretinal, skin, gastrointestinal, and/or pulmonary barriers.

### Compositions for Increasing SHANK3 Protein Levels

In the case of rare, monogenic diseases, the loss of one functional allele, e.g., SHANK3 allele, can result in haploinsufficiency and the associated disease.

While not wishing to be bound by theory, it is believed that ASOs can act by a number of mechanisms, including, but not limited to targeting sequences within the 3′ UTR of SHANK3 mRNA that can stabilize SHANK3 transcripts and/or net translation, e.g., by masking binding sites for miRNAs (e.g., a binding site for miR-504) located within the SHANK3 mRNA 3′ UTR to sterically hinder (“mask”) access of these miRNAs to their binding sites, thereby resulting in an increased level of SHANK3 mRNA and ultimately allowing increased translation of SHANK3 protein. Alternatively, alterations in the secondary structure of the 3′ UTR induced by ASO hybridization could also result in increased translation and/or stabilization of the targeted SHANK3 transcript.

Accordingly, disclosed herein is an ASO that binds within a targeted portion of the 3′ UTR of a SHANK3 mRNA; whereby binding of the antisense oligonucleotide within the targeted portion in a mammalian cell results in an increased level of SHANK3 protein in the mammalian cell.

For reference, the nucleotide sequence of the canonical human SHANK3 mRNA 3′ UTR is provided herein as SEQ ID NO:1 (provided in the Appendix). In some examples the targeted portion comprises the sequence according to SEQ ID NO:1079. In some examples the targeted portion consists of the sequence according to SEQ ID NO:1079 (provided in the Appendix).

### Antisense Oligonucleotides (ASOs) and Antisense RNAs (ARs)

In some examples of the compositions and methods described herein, ASOs and ARs have a sequence that is completely or nearly completely complementary across its length to the target sequence. ASOs and ARs are designed so that they bind (hybridize) to a target RNA sequence (e.g., a targeted portion of a mRNA transcript) and remain hybridized under physiological conditions. Selection of suitable sequences for ASOs and ARs generally avoids, where possible, similar nucleic acid sequences in other (i.e., off-target) locations in the genome or in cellular mRNAs or miRNAs, such that the likelihood the ASO or AR will hybridize at such sites is limited. In some examples, ASOs disclosed herein bind to a targeted region within the SHANK3 mRNA 3′ UTR.

In some examples, ASOs or ARs “specifically hybridize” to or are “specific” to a target nucleic acid or a targeted portion of a SHANK3 mRNA 3′ UTR. At a given ionic strength and pH, the Tₘ is the temperature at which 50% of a target sequence hybridizes to a complementary oligonucleotide.

ASO and AR sequences are “complementary” to their target sequences when hybridization occurs in an antiparallel configuration between two single-stranded polynucleotides. Complementarity is quantifiable in terms of the proportion (e.g., the percentage) of bases in opposing strands that are expected to form hydrogen bonds with each other, according to generally accepted base-pairing rules. The nucleotide sequence of an ASO or AR need not be 100% complementary to that of its target nucleic acid to hybridize. In certain examples, the nucleotide sequences of ASOs or ARs in the compositions disclosed herein can be at least 60%, at least 65%, at least 70%, at least 75%, at least 80%, at least 85%, at least 90%, at least 95%, at least 96%, at least 97%, at least 98%, or at least 99% sequence complementary to the nucleotide sequence of the targeted portion of an RNA transcript over the length of the ASO or AR nucleotide sequence. For example, an ASO or AR in which 18 of 20 nucleotides of ASO or AR sequence are complementary to a target region, and would therefore specifically hybridize, would represent 90 percent complementarity. In such an example, the remaining non-complementary nucleotides of the ASO or AR could be clustered together or interspersed with complementary nucleotides and need not be contiguous. Complementarity of an ASO or AR sequence to a target nucleotide sequence (expressed as “percent complementarity” to its target sequence; or “percent identity” to its reverse complement sequence) can be determined routinely using algorithms known in the art, as exemplified in the BLAST programs (basic local alignment search tools) and PowerBLAST programs (Altschul, et al., 1990, J Mol. Biol., 215:403-410; Zhang et al., 1997, Genome Res., 7:649-656).

In some examples, an ASO or AR does not hybridize to all nucleotides in a target sequence and the nucleotide positions at which it does hybridize may be contiguous or noncontiguous. ASOs or ARs may hybridize over one or more segments of a SHANK3 mRNA 3′ UTR such that intervening or adjacent segments (e.g., one or more abasic spacer subunits) are not involved in the hybridization event (e.g., a loop structure or hairpin structure may be formed).

In some examples the nucleotide sequence of the ASO or the AR is at least 60%, 65%, 70%, 75%, 80%, 85%, 90%, 95%, or 100% complementary to the nucleotide sequence of the targeted portion of the SHANK3 mRNA over the length of the ASO or the AR. In some examples the nucleotide sequence of the ASO or the AR comprises a sequence that: (a) has at least about 40% identity to about 60% identity of the nucleotide sequence of an ASO or AR sequence disclosed herein, e.g., 45% identity, 48% identity, 50% identity, 52% identity, 55% identity, 58% identity or another sequence identity from about 40% identity to about 60% identity to the entire length of the sequence of any ASO or AR disclosed herein; and (b) comprises a contiguous sequence of at least 8 bases to 16 bases that is 100% identical to a contiguous sequence of at least 8 to 16 bases in any one of the ASO or AR sequences disclosed herein, e.g., 100% sequence-identical to a contiguous 9 bases, 10 bases, 11 bases, 12 bases, 13 bases, 14 bases, 15 bases, or 16 bases of an ASO or AR sequence disclosed herein. The ASOs or ARs for use in the compositions described herein may be of any length suitable for specific hybridization to a target sequence. In some examples, the nucleotide sequence of the ASOs or ARs consist of 8 to 50 nucleotides. For example, the ASO or AR sequence can be 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleotides in length. In some examples, the nucleotide sequence of the ASOs or ARs consist of 8 to 50 nucleotides. For example, the ASO or AR sequence can be 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleotides in length. In some examples, the ASOs consist of more than 50 nucleotides, but no more than 100 nucleotides in length. In some examples, the ASO or AR nucleotide sequence is from 8 to 50 nucleotides, 8 to 40 nucleotides, 8 to 35 nucleotides, 8 to 30 nucleotides, 8 to 25 nucleotides, 8 to 20 nucleotides, 8 to 15 nucleotides, 9 to 50 nucleotides, 9 to 40 nucleotides, 9 to 35 nucleotides, 9 to 30 nucleotides, 9 to 25 nucleotides, 9 to 20 nucleotides, 9 to 15 nucleotides, 10 to 50 nucleotides, 10 to 40 nucleotides, 10 to 35 nucleotides, 10 to 30 nucleotides, 10 to 25 nucleotides, 10 to 20 nucleotides, 10 to 15 nucleotides, 11 to 50 nucleotides, 11 to 40 nucleotides, 11 to 35 nucleotides, 11 to 30 nucleotides, 11 to 25 nucleotides, 11 to 20 nucleotides, 11 to 15 nucleotides, 12 to 50 nucleotides, 12 to 40 nucleotides, 12 to 35 nucleotides, 12 to 30 nucleotides, 12 to 25 nucleotides, 12 to 20 nucleotides, 12 to 15 nucleotides, 13 to 50 nucleotides, 13 to 40 nucleotides, 13 to 35 nucleotides, 13 to 30 nucleotides, 13 to 25 nucleotides, 13 to 20 nucleotides, 14 to 50 nucleotides, 14 to 40 nucleotides, 14 to 35 nucleotides, 14 to 30 nucleotides, 14 to 25 nucleotides, 14 to 20 nucleotides, 15 to 50 nucleotides, 15 to 40 nucleotides, 15 to 35 nucleotides, 15 to 30 nucleotides, 15 to 25 nucleotides, 15 to 20 nucleotides, 17 to 30 nucleotides, 17 to 25 nucleotides, 17 to 20 nucleotides, 20 to 50 nucleotides, 20 to 40 nucleotides, 20 to 35 nucleotides, 20 to 30 nucleotides, 20 to 25 nucleotides, 25 to 50 nucleotides, 25 to 40 nucleotides, 25 to 35 nucleotides, or 25 to 30 nucleotides in length. In some examples, the ASOs or ARs are 17 nucleotides in length. In other examples, the ASOs or ARs are 20 nucleotides in length. In some examples, the nucleotide sequence of the ASO or AR nucleotide is 25 nucleotides in length.

In other examples the ASOs or ARs comprise at least 10 contiguous nucleotides of an ASO or AR sequence described herein. In some examples ASOs or ARs comprise at least 10 contiguous nucleotides (subsequence) from each of two or more ASO or AR sequences described herein, where the two or more subsequences are not contiguous in a SHANK3 mRNA sequence.

In some examples for each occurrence of “G” in an ASO or AR sequence disclosed herein, the “G” is guanosine or inosine. In some examples for each occurrence of “T” in an ASO or AR sequence disclosed herein, the “T” is any one of: thymidine, inosine, uracil, or an isomeric or modified form of uracil (e.g., pseudouridine or N1-methyl-pseudouridine). In some examples for each occurrence of “C” in an ASO or AR sequence disclosed herein, the C is cytosine or a modified form of cytosine (e.g., 5′-methyl cytosine).

In some examples the nucleotide sequence of the ASO corresponds to any one of SEQ ID NOs:1080-1093 or 1096-1133. In some examples the nucleotide sequence of the ASO corresponds to any one of SEQ ID NOs:1080-1093. In some examples the nucleotide sequence of the ASO corresponds to any one of SEQ ID NOs:1084, 1087, or 1091-1093. In some examples the nucleotide sequence of the ASO corresponds to SEQ ID NO:1084. In some examples the nucleotide sequence of the ASO corresponds to SEQ ID NO:1087. In some examples the nucleotide sequence of the ASO corresponds to SEQ ID NO:1091. In some examples the nucleotide sequence of the ASO corresponds to SEQ ID NO:1092. In some examples the nucleotide sequence of the ASO corresponds to SEQ ID NO:1093.

In some examples any of the foregoing ASOs include a backbone modification. In some examples the backbone modification includes a phosphorothioate linkage or a phosphorodiamidate linkage. In other examples the ASO includes a phosphorodiamidate morpholino, an arabinonucleic acid, a locked nucleic acid, a peptide nucleic acid, a 2′-O-modification such as a 2′-O-methyl, a 2′-Fluoro, or a 2′-O-methoxyethyl moiety, or an abasic spacer subunit. In some examples the abasic spacer subunit comprises the structure according to Formula I.

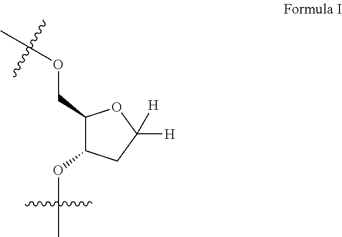 [SMILES: *OC[C@H]1OCC[C@@H]1O*]

In some examples the nucleotide sequence of the ASO comprises one or more abasic spacer subunit and/or one or more locked nucleic acids (LNAs). In some examples the nucleotide sequence of the ASO comprises one or more abasic spacer subunits and one or more LNAs. In some examples, where the ASO comprises at least one abasic spacer subunit, at least one locked nucleic acid, or both, the nucleotide sequence of the ASO corresponds to any one of SEQ ID NOs:1096-1133.

In some examples the ASO includes at least one modified sugar moiety. In other examples each sugar moiety in the ASO is a modified sugar moiety. In some examples the ASO includes a 2′-O-methoxyethyl moiety. In other examples each nucleotide of the ASO includes a 2′-O-methoxyethyl moiety.

In some examples the nucleotide sequence of the ASO or AR comprises the sequence of any one of SEQ ID NOs:3-1078. In some examples the nucleotide sequence of the ASO or AR comprises the sequence of SEQ ID NO:160 or SEQ ID NO:687. In some examples the nucleotide sequence of the ASO or AR comprises the sequence of SEQ ID NO:160. In some examples the nucleotide sequence of the ASO or AR comprises the sequence of SEQ ID NO:687. In some examples the nucleotide sequence of the ASO comprises the nucleotide sequence of SEQ ID NO:160. In other examples the nucleotide sequence of the ASO comprises the nucleotide sequence of SEQ ID NO:160.

In some examples the nucleotide sequence of the ASO or AR consists of the nucleotide sequence of any one of SEQ ID NOs:3-1078. In some examples the nucleotide sequence of the ASO or AR consists of the nucleotide sequence of SEQ ID NO:160 or SEQ ID NO:687. In some examples the nucleotide sequence of the ASO or AR consists of SEQ ID NO:160. In some examples the nucleotide sequence of the ASO or AR consists of SEQ ID NO:687. In some examples the nucleotide sequence of the ASO consists of SEQ ID NO:160. In some examples the nucleotide sequence of the ASO consists of SEQ ID NO:687. Sequences for the foregoing SEQ ID NOs are provided in Tables 1-3 in the Appendix.

### ASO Chemistry and Modifications

The ASOs used in the compositions described herein may comprise naturally-occurring nucleotides, nucleotide analogues, modified nucleotides, or any combination thereof. The term “naturally occurring nucleotides” includes deoxyribonucleotides and ribonucleotides. The term “modified nucleotides” includes nucleotides with modified or substituted sugar groups and/or having a modified backbone. In some examples, all the nucleotides of an ASO are modified nucleotides. Chemical modifications of ASOs or components of ASOs that are compatible with the compositions and methods described herein are known in the art as disclosed in, e.g., in U.S. Pat. Nos. 8,258,109, 5,656,612, U.S. Patent Publication No. 2012/0190728, and Roberts et al., 2020, Nature Rev. Drug Disc., 19:673-694.

One or more nucleotides of an ASO may be any naturally occurring, unmodified nucleobase such as adenine, guanine, cytosine, thymine, uracil and inosine, or any synthetic or modified nucleobase that is sufficiently similar to an unmodified nucleobase such that it is capable of hydrogen bonding with a nucleobase present on a target RNA transcript. Examples of suitable modified nucleobases include, but are not limited to, hypoxanthine, xanthine, 7-methylguanine, 5, 6-dihydrouracil, 5-methylcytosine, and 5-hydroxymethylcytosine.

In some examples an ASO includes 1, 2, or 3 abasic spacer subunits, residues that comprise a sugar backbone but do not include a nucleobase, interspersed at various positions within an ASO sequence. Abasic “spacer” subunits and their use in oligonucleotides is described in, e.g., PCT/US2022/044995. In some preferred examples the abasic spacer subunit comprises the structure according to Formula I:

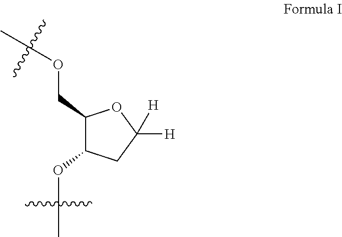 [SMILES: *OC[C@H]1OCC[C@@H]1O*]

In some examples an abasic spacer subunit comprises the structure according to Formula II:

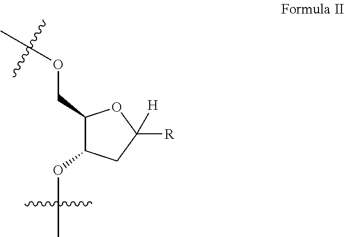

In some examples an ASO disclosed herein includes 1-3 abasic spacer subunits and 1-3 locked nucleic acid subunits.

ASOs include a “backbone” structure, that refers to the connection between nucleotides/monomers of the ASO. In naturally occurring oligonucleotides, the backbone comprises a 3′-5′ phosphodiester linkage connecting sugar moieties of adjacent nucleotides. Suitable types of backbone linkages for the ASOs described herein include, but are not limited to, phosphodiester, phosphorothioate, phosphorodithioate, phosphorodiamidate, phosphoroselenoate, phosphorodiselenoate, phosphoroanilothioate, phosphoraniladate, phosphoramidate, and the like. In some examples, the backbone modification is a phosphorothioate linkage. In other examples, the backbone modification is a phosphorodiamidate linkage. See, e.g., Roberts et al. supra; and Agrawal (2021), Biomedicines, 9:503. In some examples, the backbone structure of the ASO does not contain phosphorous-based linkages, but rather contains peptide bonds, for example in a peptide nucleic acid (PNA), or linking groups including carbamate, amides, and linear and cyclic hydrocarbon groups.

In some examples, the stereochemistry at each of the phosphorus internucleotide linkages of the ASO backbone is random. In other examples, the stereochemistry at each of the phosphorus internucleotide linkages of the ASO backbone is controlled and is not random. For example, U.S. Pat. No. 9,605,019 describes methods for independently selecting the handedness of chirality at each phosphorous atom in an oligonucleotide. In some examples, an ASO used in the compositions and methods provided herein, including, but not limited to, the ASOs the sequences of which are disclosed herein as SEQ ID NOs:1080-1093, 1096-1133, SEQ ID NOs:3-1078 (e.g., SEQ ID NO:160 or SEQ ID NO:687), and SEQ ID NOs: 1080-1133. In some examples, a composition or composition used in the methods disclosed herein comprises a pure diastereomeric ASO. In other examples, the composition comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some examples, the ASO has a non-random mixture of Rp and Sp configurations at its phosphorus internucleotide linkages. In some examples, an ASO used in the compositions and methods disclosed herein, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp.

In some examples, the ASOs described herein contain a sugar moiety that comprises ribose or deoxyribose, or a modified sugar moiety or sugar analogue, including a morpholine ring. Suitable examples of modified sugar moieties include, but are not limited to, 2′ substitutions such as 2′-O-modifications, 2′-O-methyl (2′-O-Me), 2′-O-methoxyethyl (2′MOE), 2′-O-aminoethyl, 2′F, N3′->P5′ phosphoramidate, 2′dimethylaminooxyethoxy, 2′dimethylaminoethoxyethoxy, 2′-guanidinidium, 2′-O-guanidinium ethyl, 2′-deoxy-2′-fluoroarabinonucleic acid (2′FANA), carbamate modified sugars, and bicyclic modified sugars. In some examples, the sugar moiety modification is selected from among 2′-O-Me, 2′F, and 2′MOE. In other examples, the sugar moiety modification is an extra bridge bond, such as in a locked nucleic acid (LNA). In some examples the sugar analogue contains a morpholine ring, such as phosphorodiamidate morpholino (PMO). In some examples, the sugar moiety comprises a ribofuransyl or 2′deoxyribofuransyl modification. In some examples, the sugar moiety comprises 2′4′-constrained 2′-O-methyloxyethyl (cMOE) modifications. In some examples, the sugar moiety comprises cEt 2′, 4′ constrained 2′-O ethyl BNA modifications. In other examples, the sugar moiety comprises tricycloDNA (tcDNA) modifications. In some examples, the sugar moiety comprises ethylene nucleic acid (ENA) modifications. In some examples, the sugar moiety comprises 2′-O-(2-N-methylcarbamoylethyl) (MCE). Modifications are known in the art as exemplified in Jarver, et al., 2014, Nucleic Acid Therapeutics, 24(1): 37-47.

In some examples, each constituent nucleotide of the ASO is modified in the same way, e.g., every linkage of the backbone of the ASO comprises a phosphorothioate linkage, or each ribose sugar moiety comprises a 2′-O-methyl modification. In other examples, a combination of different modifications is used, e.g., an ASO comprising a combination of phosphorodiamidate linkages and sugar moieties comprising morpholine rings (morpholinos).

In some examples, the ASO comprises one or more backbone modifications. In some examples, the ASO comprises one or more sugar moiety modification. In some examples, the ASO comprises one or more backbone modifications and one or more sugar moiety modifications. In some examples, the ASO comprises a 2′MOE modification and a phosphorothioate backbone. In some examples, the ASO comprises a peptide nucleic acid (PNA).

In some preferred examples, the ASO comprises a phosphorodiamidate morpholino (PMO).

The skilled person in the art will appreciate that ASOs may be modified in order to achieve desired properties or activities of the ASO or reduce undesired properties or activities of the ASO. In some examples, an ASO is modified to alter one or more properties. For example, such modifications can: enhance binding affinity to a target sequence on a pre-mRNA transcript; reduce binding to any non-target sequence; reduce degradation by cellular nucleases (e.g., RNase H); improve uptake of an ASO into a cell and/or particular subcellular compartments; alter the pharmacokinetics or pharmacodynamics of the ASO; and/or modulate the half-life of the ASO in vivo.

In some examples, the ASOs comprise one or more 2′-O-(2-methoxyethyl) (MOE) phosphorothioate-modified nucleotides, which have been shown to confer significantly enhanced resistance of ASOs to nuclease degradation and increased bioavailability.

In some examples the ASOs comprise one or more abasic spacer subunits, which may reduce off-target hybridization of the ASO.

Methods for synthesis and chemical modification of ASOs, as well as synthesis of ASO conjugates is well known in the art, and such ASOs are available commercially.

In some examples, a composition (e.g., a pharmaceutical composition) provided here includes two or more ASOs with different chemistries but complementary to the same targeted portion of the SHANK3 mRNA 3′ UTR. In other examples, a composition comprises two or more ASOs that are complementary to different targeted portions of the 3′ UTR.

In some examples, the compositions disclosed herein include ASOs that are linked to a functional moiety. In some examples, the functional moiety is a delivery moiety, a targeting moiety, a detection moiety, a stabilizing moiety, or a therapeutic moiety. In some examples the functional moiety includes a delivery moiety or a targeting moiety. In some examples the functional moiety includes a stabilizing moiety. In some preferred examples the functional moiety is a delivery moiety.

Suitable delivery moieties include, but are not limited to, lipids, polyethers, peptides, carbohydrates, glycans, receptor binding peptide (RBP), and antibodies.

In some examples, the delivery moiety includes a cell-penetrating peptide (CPP). Suitable examples of CPPs are described in, e.g., PCT/AU2020/051397. In some examples the amino acid sequence of the CPP comprises or consists of: RRSRTARAGRPGRNSSRPSAPR (SEQ ID NO:2). In other examples, the delivery moiety includes a RBP.

In other examples, the delivery moiety includes a carbohydrate. In some examples, a carbohydrate delivery moiety is selected from among N-acetylgalactosamine (GalNAc), N-Ac-Glucosamine (GluNAc), glycan, and a mannose. In one example, the carbohydrate delivery moiety comprises GalNac or a glycan moiety.

In other examples, the delivery moiety includes a lipid. In some examples the lipid contains saturated acids. In some examples the lipid contains unsaturated acids. In some examples the lipid contains saturated and unsaturated acids. Examples of suitable lipids as delivery moieties include, but are not limited to, cholesterol moiety, a cholesteryl moiety, and aliphatic lipids. In some examples the delivery moiety includes a fatty acid or lipid moiety. In some embodiments the fatty acid chain length is about C8 to C20. Examples of suitable fatty acid moieties and their conjugation to oligonucleotides are found in, e.g., International Patent Publication WO 2019232255 and in Prakash et al., (2019).

In further examples, the delivery moiety includes an antibody, as described in, e.g., Dugal-Tessier et al., (2021).

Suitable examples of stabilizing moieties include, but are not limited to, polyethylene glycol (PEG), poly(oligo(ethylene glycol) methyl ether methacrylate) (POEGMA), and Poly(2-oxazoline)s (POx).

In some examples, where an ASO is linked to a functional moiety, the functional moiety is covalently linked to the ASO. In other examples, the functional moiety is non-covalently linked to the ASO.

Functional moieties can be linked to one or more of any nucleotides in an ASO at any of several positions on the sugar, base or phosphate group, as understood in the art and described in the literature, e.g., using a linker. Linkers can include a bivalent or trivalent branched linker. In some examples, the functional moiety is linked to the 5′ end of the ASO. In other examples, the functional moiety is linked to the 3′ end of the ASO.

In some examples compositions comprising any of the ASOs disclosed herein also include a delivery nanocarrier complexed with ASO. In some examples, a delivery nanocarrier is selected from among lipoplexes, liposomes, exosomes, inorganic nanoparticles, and DNA nanostructures. In other examples the delivery nanocarrier includes a lipid nanoparticle encapsulating the ASO. Various delivery ASO-nanocarrier complex formats are known in the art, as reviewed in, e.g., Roberts et al., supra.

### Vectors for Expression of SHANK3 Antisense RNA (AR)

In some examples provided herein is a vector for expression, in a mammalian neuron or other cell type, of an antisense RNA (AR) that binds within a targeted portion of the 3′ UTR of a SHANK3 mRNA; whereby binding of the AR within the targeted portion in a mammalian cell results in an increased level of SHANK3 protein in the mammalian cell.

In some examples, the promoter used in the expression vector is a neuron type-selective promoter for driving expression of the AR in the mammalian cell. In some examples, the neuronal cell type-selective promoter is selective for expression in neurons selected from the list consisting of. cortical, striatal, cerebellar and hippocampal excitatory or inhibitory neurons, including, but not limited to cortical glutamatergic neurons, cortical GABAergic neurons, hippocampal glutamatergic neurons, and striatal inhibitory neurons.

In some the promoter is an inducible promoter, e.g., inducible by a ligand-regulated transactivator such as the tet-inducible rtTA, which allows titration of AR transcription in a target mammalian cell. In some examples, the promoter driving AR expression is a U6 or other Pol III promoter, which is particularly suitable for transcription of short RNA sequences such AR sequences disclosed herein. In some examples, an expression vector utilizes hybrid promoter systems, e.g., a Tet-O-regulated U6 promoter system as described in Lin et al. (2004), FEBS Letters, 577 (2004) 376-380. In some examples, where both cell type-specificity and inducibility of an AR expression vector are desired, a two-part expression system is used in which expression of a ligand-regulated transactivator is driven by a cell type-selective promoter and expression of an AR disclosed herein is driven by a promoter regulated by the ligand-regulated transactivator.

In some examples, the expression vectors used in the compositions disclosed herein are non-viral expression vectors, e.g., plasmid vectors, minicircle DNA vectors, linear amplicon expression cassettes, and the like.

In some examples, composition containing a non-viral expression virus further comprises a transfection agent. Exemplary transfection agents for transfection include, but are not limited to, jet-PEI® (available from Polyplus-Transfection® SA, Strasbourg, France); TurboFect in vivo Transfection Reagent (ThermoFisher), and cationic derivatives of polyisoprenoid alcohols (PTAI) as described in, e.g., Rak et al., (2016).

In other examples, the expression vectors to be used are viral vectors, i.e., non-replicative recombinant viruses suitable for expression of an AR disclosed herein.

Preferably, the recombinant virus for expression of the SHANK3 AR is a DNA virus. Suitable types of DNA viruses include adeno-associated virus (AAV), adenovirus, lentivirus, herpes simplex virus (HSV), and anelloviruses. Methods for design, production, and use of such types of recombinant DNA viruses are established in the art, as exemplified in Fukazawa et al., (2010), International J of Mol. Med, 25(1), 3-10, and in “Gene Therapy Protocols” for adenovirus; “Adeno-Associated Virus: Methods and Protocols” for AAV; Cody et al (2013), Journal of Genetic Syndromes & Gene Therapy, 4(1), 126, and “Herpes Simplex Virus: Methods and Protocols” for HSV; “Gene Therapy Protocols Vol. 1: Production and In Vivo Applications of Gene Transfer Vectors”; and Merten et al. (2016), Molecular Therapy—Methods & Clinical Development, 3, 16017, and Emeagi et al. (2013), Current Molecular Medicine 13(4), 602-625 for lentivirus. In some preferred examples, the viral vector is a recombinant AAV.

### Genetically Modified Cells

Also provided herein are genetically modified cells. In some examples the genetically modified cells are genetically modified bacterial cells (e.g., recombinant E. coli, for amplifying an AR expression vector disclosed herein). In other examples the genetically modified cells are mammalian cells that become genetically modified by virtue of the fact that they have been transfected with any of the ASOs or non-viral AR expression vectors; or transduced with any of the viral AR expression vectors disclosed herein. In some examples, the genetically modified mammalian cells are ex vivo, e.g., as a cultured cell population. In other examples, the genetically modified mammalian cells are in vivo, e.g., in a mouse. In some examples, the genetically modified mammalian cells are human cells.

In some examples the genetically modified mammalian cells are neurons or neural progenitors. Suitable examples of neurons include, but are not limited to, cortical, striatal, cerebellar and hippocampal excitatory or inhibitory neurons, including, but not limited to cortical glutamatergic neurons, cortical GABAergic neurons, hippocampal glutamatergic neurons, and striatal inhibitory neurons. In some examples such primary cell types can be obtained by differentiation of a human pluripotent stem cell line, e.g., an hiPSC line or a human embryonic stem cell (hESC) line. Methods for obtaining a variety of different neuronal cell types is known in the art, as reviewed in, e.g., Alia et al., (2019), Fitzgerald et al., (2020) and Kim et al., (2014). In other examples, the genetically modified mammalian cells are derived from a cell line. In some examples the cell line is pluripotent stem cell line (e.g., hiPSCs or hESCs) or a neuronal cell line. Suitable neuronal or neuronal stem cell lines include, but are not limited to, SH-SY5Y, NTera, CTXOE16, ReNcell VM, ReNcell Cx. In some preferred examples, the genetically modified mammalian cells express SHANK3 endogenously.

The genetically modified cells disclosed herein can be genetically modified by any of a number of methods and strategies known in the art, e.g., transient transfection, stable transfection, and viral transduction. In some examples transfection with ASOs or non-viral vectors is carried out by nucleofection. In other examples transfection of cells is by lipofection.

### Pharmaceutical Compositions

Also provided herein are pharmaceutical compositions comprising any of the foregoing ASOs, non-viral expression vectors, and viral expression vectors disclosed herein, and formulated with at least a pharmaceutically acceptable excipient, including a carrier, filler, preservative, adjuvant, solubilizer and/or diluent.

Pharmaceutical compositions containing any of the ASOs or expression vector compositions described herein, for use in the methods disclosed herein, can be prepared according to conventional techniques well known in the pharmaceutical industry and described in the published literature. In some examples, a pharmaceutical composition for treating a subject comprises a therapeutically effective amount of any ASO or expression vector disclosed herein.

Pharmaceutically acceptable salts are suitable for use in contact with the tissues of humans and lower animals without undue toxicity, irritation, allergic response, etc., and are commensurate with a reasonable benefit/risk ratio. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some examples, pharmaceutical compositions are formulated into any of a number of possible dosage routes or forms including, but not limited to, intravenous administration, intrathecal administration magna administration, tablets, capsules, gel capsules, liquid syrups, and soft gels. In some examples, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some examples, a pharmaceutical formulation disclosed herein is provided in a form including, but not limited to, a solution, emulsion, microemulsion, foam or liposome-containing formulation (e.g., cationic or noncationic liposomes).

In some examples, pharmaceutical formulations comprising any of the ASOs or expression vectors described herein may comprise one or more penetration enhancers, carriers, excipients or other active or inactive ingredients as appropriate and known to the skilled person. In some examples, where a pharmaceutical composition includes liposomes, such liposomes can also include sterically stabilized liposomes, e.g., liposomes comprising one or more specialized lipids. These specialized lipids result in liposomes with enhanced circulation lifetimes. In some examples, a sterically stabilized liposome comprises one or more glycolipids or is derivatized with one or more hydrophilic polymers, such as PEG moiety. In some examples, a surfactant is included in the pharmaceutical formulation.

In some examples, a pharmaceutical composition also includes a penetration enhancer to enhance the delivery of ASOs or non-viral expression vectors, e.g., to aid diffusion across cell membranes and/or enhance the permeability of a lipophilic drug. In some examples, the penetration enhancers include a surfactant, a fatty acid, a bile salt, or a chelating agent.

In some examples, where administration is via a systemic route, e.g., intravenous, the method also includes a step to facilitate transfer of any of the ASOs or vectors described herein across the blood brain barrier (BBB) into the CNS, and especially into the brain. In some examples the BBB is transiently disrupted, e.g., by administration of one or more antibodies that disrupt Netrin-1 binding to Unc5B as described in Boye et al., (2022).

In some examples, a pharmaceutical composition comprises a dose of ASOs or non-viral vectors ranging from about 0.01 mg/kg to 20 mg/kg, e.g., 0.05 mg/kg, 0.1 mg/kg, 0.2 mg/kg, 0.5 mg/kg, 1 mg/kg, 3 mg/kg, 5 mg/kg, 8 mg/kg, 10 mg/kg, 15 mg/kg, or another dose ranging from about 0.01 mg/kg to 20 mg/kg. In some examples, where an ASO disclosed herein is to be administered directly into the CNS or brain, e.g., by intracerebroventricular administration, the total dose ranges from about 50 mg to about 500 mg, e.g., 60 mg, 70 mg, 80 mg, 100 mg, 120 mg, 150 mg, 180 mg, 200 mg, 220 mg, 250 mg, 270 mg, 290 mg, 300 mg, 350 mg, 400 mg, 450 mg, or another dose from about 50 mg to about 500 mg. This dose range corresponds to approximately 0.050 mg/cm³ of brain volume to about 0.42 mg/cm³ of brain volume assuming an average human brain volume of about 1200 cm³.

In some examples, a pharmaceutical composition comprises multiple ASOs or AR expression vectors. In some examples, a pharmaceutical composition comprises, in addition to ASOs or AR expression vectors, another drug or therapeutic agent suitable for treatment of a subject suffering from SHANK3 haploinsufficiency.

### Methods

As described herein, a number of conditions (e.g., Phelan-McDermid syndrome) are associated with insufficient levels of functional SHANK3. Accordingly, the methods described herein include a method for preventing or treating a condition associated with SHANK3 haploinsufficiency by administering to the subject a therapeutically effective amount of a pharmaceutical composition comprising any of the ASOs or expression vectors disclosed herein. Likewise, in some examples, any of the ASOs or AR expression vectors disclosed herein are used in the manufacture of a medicament for treating a condition associated with SHANK3 haploinsufficiency. In some examples the condition associated with SHANK3 haploinsufficiency to be prevented or treated by the methods or with the compositions disclosed herein is Phelan-McDermid syndrome, an autism spectrum disorder, schizophrenia, or an intellectual disability. In some preferred examples, the condition is Phelan-McDermid syndrome.

Also provided herein is a method for increasing the amount of functional SHANK3 protein in a mammalian cell expressing SHANK3 mRNA, the method comprising contacting the cell with any of the ASOs or expression vectors disclosed herein.

In some examples, administration to a subject or contact with cells in vitro or ex vivo with any of the ASOs, AR expression vectors, or pharmaceutical compositions disclosed herein increases the level of SHANK3 protein about 1.1 to about 5 fold in cells (e.g., neurons) in vitro or ex vivo, e.g., 1.2 fold, 1.3 fold, 1.5 fold, 1.7 fold, 2 fold, 2.2 fold, 2.5 fold, 2.7 fold, 3 fold, 3.3 fold, 3.5 fold, 4 fold, 4.3 fold, 4.5 fold, 4.7 fold, or another increase in SHANK3 protein levels from about 1.1 fold to about 5 fold in cells in a subject or in vitro or in vivo.

Suitable routes of administration for treatment with the compositions, pharmaceutical compositions, or medicaments disclosed herein include, but are not limited to, intravenous, intra-arterial, intraparenchymal, intracerebroventricular, intra-cistema magna, intrathecal, intravenous, intra-arterial, subcutaneous, and topical.

As the skilled person will understand, the treatment methods disclosed herein include administration of the compositions and pharmaceutical compositions disclosed herein in a therapeutically effective amount to a subject (e.g., a human subject). The terms “effective amount” or “therapeutically effective amount,” as used herein, refer to a sufficient amount of a disclosed ASO, non-viral or viral expression vector being administered to relieve to some extent one or more of the symptoms and/or clinical indicia associated with SHANK3 haploinsufficiency in a particular disease or health condition. In some examples, an “effective amount” for therapeutic uses is the amount of one of the foregoing agents required to provide a clinically significant decrease in disease symptoms to prevent disease symptoms without undue adverse side effects. Examples of suitable symptoms to be reduced by the treatment methods provided herein included, but are not limited to, seizures, anxiety, repetitive behaviors, learning and memory deficits, and impaired sociability. An appropriate “effective amount” in any individual case may be determined using techniques, such as a dose escalation study. The term “therapeutically effective amount” includes, for example, a prophylactically effective amount. It is understood that “an effective amount” or “a therapeutically effective amount” can vary from subject to subject, due to variation in metabolism of the compound of any age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

### Combination Treatments

The pharmaceutical compositions comprising any of the ASOs or AR expression vectors, disclosed herein, can also be used in combination with other agents of therapeutic value in the treatment of a condition associated with SHANK3 haploinsufficiency. In general, other agents do not necessarily have to be administered in the same pharmaceutical composition, and may, because of different physical and chemical characteristics, preferably be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

Compositions and pharmaceutical compositions comprising ASOs and/or expression vectors, and an additional therapeutic agent may be administered concurrently (e.g., simultaneously, essentially simultaneously or within the same treatment protocol) or sequentially, depending upon the stage and progression of the SHANK3 haploinsufficiency-associated condition to be treated, the condition of the patient, and the choice of specific therapeutic agents used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies, dosages of co-administered therapeutic agents will of course vary depending on the type of co-agents employed, ASO or expression vector, and the disease stage of the patient to be treated.

Pharmaceutical compositions comprising ASOs, ARs, or expression vectors, and an additional therapeutic agent that make up a combination therapy disclosed herein may be a combined dosage form or in separate dosage forms intended for substantially simultaneous administration. The pharmaceutical compositions that make up the combination therapy may also be administered sequentially, with either therapeutic agent being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of various physiological parameters may also be evaluated to determine the optimal dose interval.

Examples of suitable therapeutic agents for co-administration with a composition or a pharmaceutical composition disclosed herein include, but are not limited to, Growth Hormone, Insulin-Like Growth Factor-1, Risperidone, Lumateperone, sodium valproate, lithium, and D-serine.

### EXAMPLES

### Example 1: Identification of SHANK3 Target Sequences in the 3′ UTR

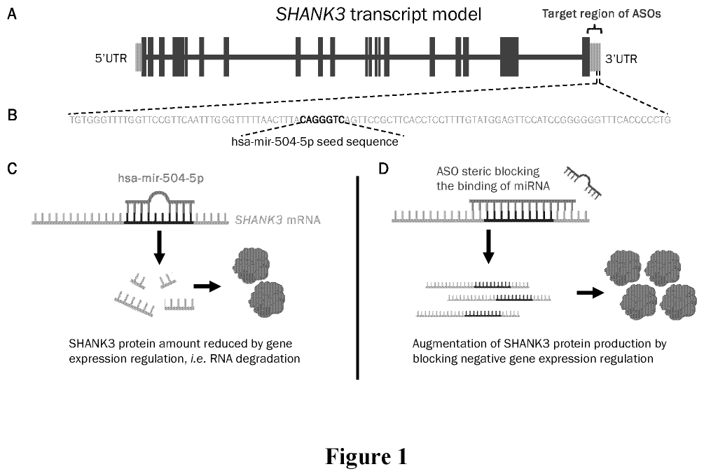

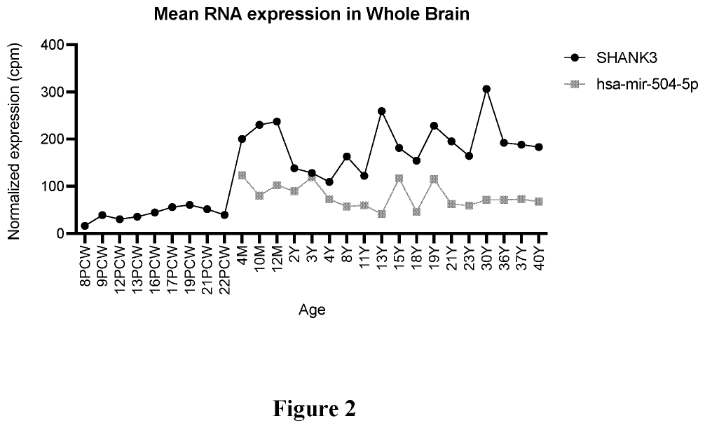

ASO sequences are designed as 1 nucleotide microwalks of SHANK3 3′ UTR target regions of interest. Matured sequences are generated to optimize therapeutic outcomes utilizing two approaches; employment of advanced algorithms to design mismatches that improve the ASO's neurotoxicity score and systematically introduce mismatches to enhance general tolerability and manufacturability. The resulting ASO sequences correspond to SEQ ID NOs:3-1078, which are provided in Table 1 (Appendix) and in the accompanying sequence listing; and SEQ ID NOs:16254-18750 provided in the accompanying sequence listing. FIG. 1 illustrates a potential mechanism of antisense oligonucleotides by masking binding sites for miRNAs (e.g., a binding site for miR-504) located within the SHANK3 mRNA 3′ UTR and prevent access of these miRNAs to their binding sites, thereby resulting in an increased level of SHANK3 gene expression. For RNA seq analysis of SHANK3 mRNA and miR-504, bulk-RNA-seq raw data was downloaded from the publicly available BrainSpan data repository. Fastq files were quality checked and adapter trimmed using the fastp tool. Then, processed reads were mapped against the T2T genome reference and the lift off gene annotation from Gencode v45. Transcript quantification was performed with Salmon tool using the same lift off gene annotation mentioned before. Transcript quantification was transformed to gene counts using the tximeta R tool. Gene expression analysis was performed in R with edgeR. Samples with <10 million reads were discarded, and data was normalized with the TMM edgeR method. MicroRNA filtered counts from the BrainSpan Small RNA-seq data was obtained from PsychENCODE processed data repository. MicroRNA counts were normalized and expression analysed using edgeR tool in R. FIG. 2 demonstrates the expression of miR-504 (hsa-mir-504-5p) and SHANK3 mRNA expression in human brain samples by RNAseq.

### Example 2: Screening of Peptide-Conjugated Phosphorodiamidate Morpholinos (PPMOs) in a Neuronal Cell Line

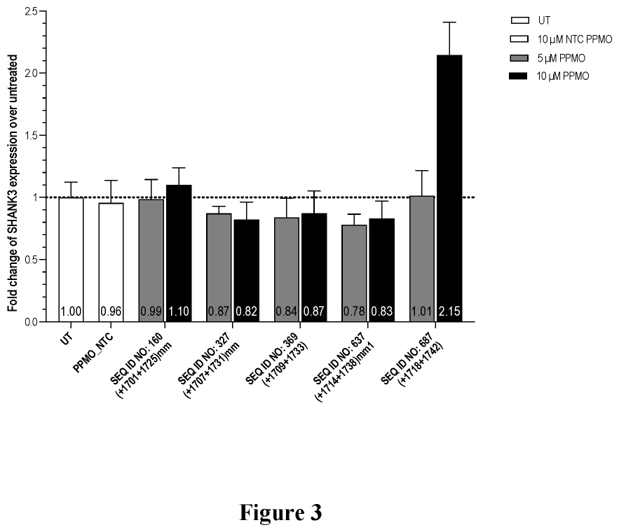

PMO sequences corresponding to (SEQ ID NOs:160, 327, 369, 637, and 687) in Example 1 were conjugated to a cell penetrating peptide (SEQ ID NO: 2) to generate PPMOs and assessed in SH-SY5Y cells for their ability to increase SHANK3 protein expression levels. PPMO were applied to SH-SY5Y neuronal cell line cultures at concentrations of 5 μM and 10 μM and incubated for 5 days. Five days after PPMO treatment, total protein was extracted on ice using RIPA buffer supplemented with 1% protease inhibitor cocktail (Sigma, cat no P8340) and 1×PhosSTOP (Sigma, cat no 4906837001). Protein lysates were cleared by centrifugation, and total protein was quantitated using BCA protein kits (ThermoFisher Scientific, cat no 23225). Expression levels of SHANK3 protein were quantified by Enzyme Linked Immunosorbent Assay (ELISA). Samples were diluted in phosphate buffered saline (PBS), pH 7.4 (1 part lysate+19 parts PBS) and further diluted in 5% RIPA buffer in PBS as necessary, and standard curves were prepared in 5% RIPA buffer in PBS to match. Diluted samples and standards were loaded onto pre-coated human SHANK3 ELISA plates, and the assays were performed as directed by the manufacturer (FineTest, Cat no. EH4462). The absorbances at 450 nm were measured using a VICTOR® Nivo™ multimode plate reader, and quantitative analysis was performed using GraphPad Prism Ver 10.2.3 software. A non-targeting control, predicted not to hybridize to human transcripts, was included as a negative control. Results were first normalized to total protein concentration and then expressed as fold-change relative to untreated cells (UT). FIG. 3 demonstrates the efficacy of PPMOs in inducing SHANK3 protein upregulation. The results showed that the PPMO having an oligonucleotide sequence corresponding to SEQ ID NO:687 significantly upregulates SHANK3 protein up to two fold in SH-SY5Y cells.

### Example 3: Effect of a Selected PPMO on SHANK3 Protein Levels in Human iPSC-Derived Neurons

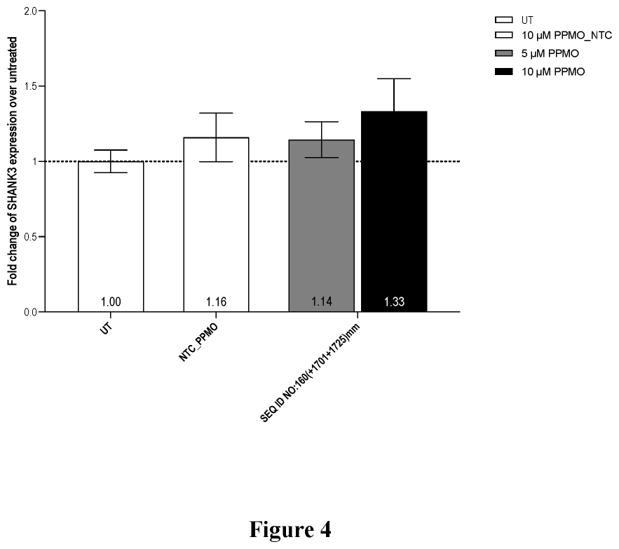

A peptide-conjugated PMO (PPMO) having the nucleotide sequence corresponding to SEQ ID NO:160 was selected to test its efficacy in iPSC-derived glutamatergic neurons derived from a healthy control subject (Cell ID: 1332iso). The iPSC-derived glutamatergic neurons were incubated with the PPMO for seven days. At day 7 post-treatment, total protein was extracted, cleared, and quantified as previously described in Example 2. The upregulation level of SHANK3 protein was quantitated using Enzyme Linked Immunosorbent Assay (ELISA). Samples were pre-diluted in phosphate buffered saline (PBS), pH 7.4 (1 part lysate+19 parts PBS) and standard curves were prepared in 5% RIPA buffer in PBS to match. Diluted samples and standards were loaded onto pre-coated human SHANK3 ELISA plates, and the assays were performed as directed by the manufacturer (FineTest, cat no EH4462). The absorbances at 450 nm were measured using a VICTOR® Nivo™ multimode plate reader, and quantitative analysis was performed using GraphPad Prism Ver 10.2.3 software. A non-targeting control, predicted not to hybridize to human transcripts, was included as a negative control. Results were first normalized to total protein concentration and then expressed as fold-change relative to UT. FIG. 4 present the assessment of SHANK3 protein expression at day 7 post PPMO treatment. The results demonstrate 1.3 fold SHANK3 protein upregulation following 10 μM PPMO treatment in iPSC-derived glutamatergic neurons.

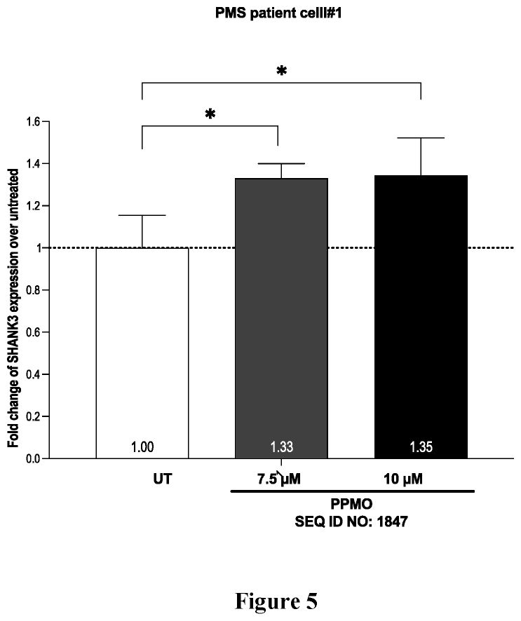

In a subsequent experiment, a PPMO having an oligonucleotide sequence corresponding to SEQ ID NO:1847 disclosed in WO 2023/235915, and having CPP as in examples above (SEQ ID NO:2) was tested in Phelan-McDermid Syndrome (PMS) patient hiPSC-derived glutamatergic neurons. The PPMO was incubated with iPSC-derived glutamatergic neurons in triplicate for 7 days. A non-targeting control, predicted not to hybridize to human transcripts, was included as a negative control. FIG. 5 shows a bar graph representing mean+S.D. of SHANK3 protein normalized to total protein amount loaded relative that of untreated cells. n=1 biological replicate. UT=untreated cells. Graph was generated using GraphPad Prism Ver 10.2.3 software. *p<0.05, **p<0.005, ***p<0.001. One-Way ANOVA. The PPMO was found to increase levels of SHANK3 protein in PMS glutamatergic neurons to a level similar to those found in control neurons.

SHANK3 has six main isoforms encoded by six different promoters located along the gene. There are five main isoforms well characterized in the brain including a, c, d, e and f (Monteiro et al., 2017). SHANK3 protein isoform ‘a’ is the full length protein of ~185 kDa and contains the six main SHANK3 functional protein domains: (1) SHANK/ProSAP N-terminal (SPN) domain interacts with small GTPases of the Ras superfamily involved in the regulation of synaptic F-actin structure and dynamics and in postsynaptic signal transduction; (2) Ankyrin repeat (ANK) region binds the cytoskeletal protein α-fodrin, an adhesive junction associated protein δ-catenin, and a component of a ubiquitin ligase complex, sharpin; (3) Src homology 3 (SH3) domain directly associates with the Ca2+ channel Ca1.3; (4) PSD-95/DLG/ZO-1 (PDZ) domain is involved in a direct interaction with SAPAP1 or the GluA1 subunit of AMPA receptors; (5) Proline-rich (Pro) region harbors multiple protein interaction sites including those for homer1 and cortactin, relevant for linking of SHANK3 to other PSD scaffolds and the regulation of spinous actin dynamics; (6) Sterile alpha motif (SAM) domain facilitates oligomerization of SHANK3 within the postsynaptic density (PSD) and is required for postsynaptic targeting. SHANK3 isoform ‘c’ contains the last four proteins domains, SH3, PDZ, Pro and SAM domains, and has a mass of ~140 kDa. SHANK3 protein isoform ‘d’ has the last three protein domains (PDZ, Pro and SAM) and a mass ~127 kDa. SHANK3 protein isoform ‘e’ only carries the two last protein domain (Pro and SAM) and has a mass of ~118 kDa. Finally, SAHNK3 protein isoform ‘f’ starts within the Pro domain and includes the SAM domain, making a mass ~88 kDa. The isoforms described above are the main isoform models for a, c, d, e and f, however there are some variants of these ones with and without internal exons (Monteiro et al., supra. The molecular weight (MW) of each isoform can vary due to these isoform variants and post-transcriptional modification.

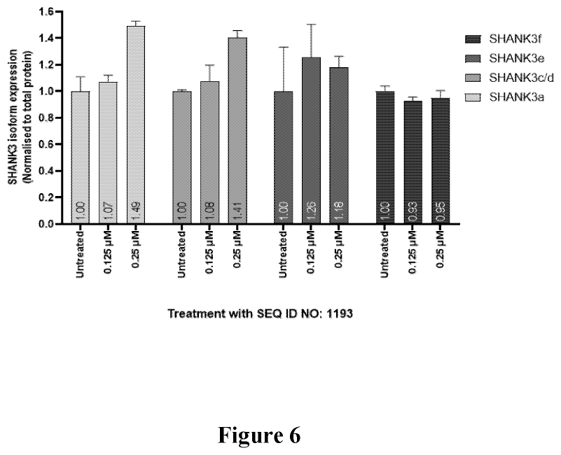

Thus, in order to assess the effect of one ASO on expression levels of some separate SHANK3 isoforms, a subsequent experiment, an ASO having the sequence corresponding to SEQ ID NO:1193 disclosed in WO 2023/235915 was tested in commercially available human iPSC-derived glutamatergic neurons (cat no: io1001, bit.bio) to determine the effect of this ASO on various SHANK3 isoforms known to be expressed in glutamatergic neurons. The iPSC-derived glutamatergic neurons were transfected with the ASO at 0.125 μM and 0.25 μM using ViaFect™ Transfection Reagent (cat no: E4981, Promega) and incubated for seven days. At day 7 post-treatment, total protein was extracted, cleared, and quantified. The upregulation level of SHANK3 protein isoforms was assessed using western blot assay. On day 7 post-treatment, protein was extracted on ice using RIPA buffer supplemented with 1% protease inhibitor cocktail (Sigma, cat no P8340) and 1× PhosSTOP (Sigma, cat no 4906837001). Protein lysates were cleared by centrifugation, and total protein was quantitated using BCA protein kits (ThermoFisher Scientific, cat no 23225). Samples were prepared by the addition of 1× Laemmli sample buffer (Bio-Rad, cat no 1610747) and Bond-Breaker™ TCEP (ThermoFisher Scientific, cat no 77720), boiled to denature proteins, then separated on NuPAGE™ 4 to 12% Bis-Tris protein gels (Invitrogen, cat no WG1401BOX). Protein was transferred to 0.2 μm nitrocellulose membranes (Bio-Rad, cat no 162112) by wet transfer overnight at 4° C. The membrane was stained for total protein (Licor, cat no 926-11021) and rabbit monoclonal anti-SHANK3 primary antibody (Cell Signaling Technology, cat no 64555S) followed by anti-rabbit secondary antibody (IRDye® 800CW preabsorbed, Abcam, cat no ab216773). Blots were imaged on an Odyssey Imager, and quantitative analysis was performed using Image Studio Ver 5.5 software. The raw fluorescence signal for each SHANK3 protein isoform was first normalized to the raw fluorescence signal of loading control (total protein). The expression level of the normalized SHANK3 protein isoform was compared to the one of the untreated control, which was set as the baseline value of 1. The expression level of the normalized SHANK3 protein isoforms was compared to those of the untreated control, which were set as the baseline value of 1. FIG. 6 shows the assessment of SHANK3 isoform protein expression at day 7 post ASO treatment determined by western blot assay. The results demonstrate approximately 1.2-1.5 fold upregulation of major SHANK3 isoforms a, c/d. Thus, while levels of each of the major isoforms evaluated were increased, the relative ratio of expression levels between the essential isoforms appears unchanged.

### Example 4: Design for the Maturation of 2′MOE ASO Sequences Targeting the 3′UTR of SHANK3 Transcript

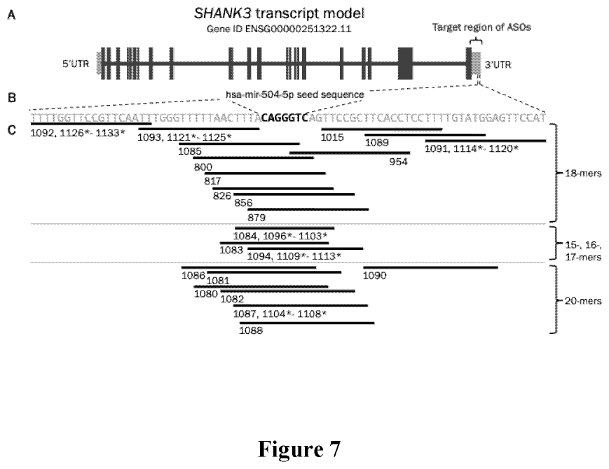

The 2′-O-methoxyethyl (2′MOE) sequences that target the SHANK3 transcript adjacent to and within the hsa-miR-504 seed sequence (SEQ ID NO: 1079) were further refined by adjusting their length (15-, 16-, 18- and 20-mers) and/or refining their target sequence to reduce GC content and improve neurotoxicity profiles. The resulting ASO sequences correspond to SEQ ID NOs:1080-1093, which are provided in Table 2 and are illustrated in FIG. 7C. FIG. 7 shows the binding sites within the target mRNA region (SEQ ID NO:1079) for ASOs with SEQ ID NOs: 1094 and 1095 (herein) which are originally disclosed in WO 2023/235915 as SEQ ID NO:1193 (“MOE55”) and SEQ ID NO:1847 (“PMO40”), respectively. ASO sequences corresponding to SEQ ID NOs.: 1080-1093, are provided in Table 2 and are illustrated in FIG. 7.

### Example 5: Screening of 2′MOE ASOs Targeting the 3′UTR of the SHANK3 Transcript

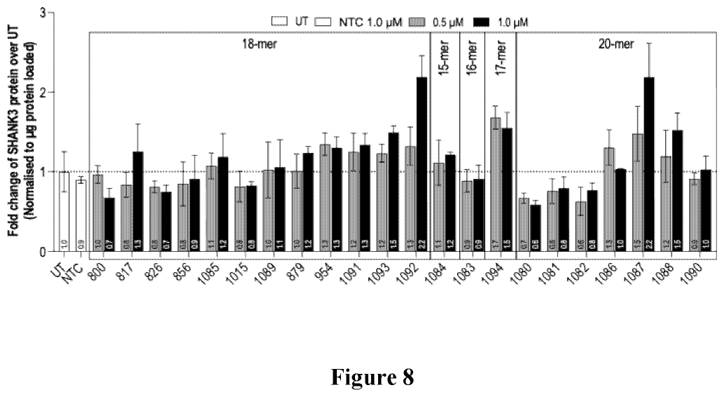

2′-O-methoxyethyl (2′MOE) ASOs corresponding to SEQ ID NOs: 800, 817, 826,856, 879, 954, 1015, and 1080-1094 were tested to assess their ability to increase SHANK3 protein expression levels. ASOs were transfected at concentrations of 0.5 μM and 1 μM to commercial iPSC-derived Cortical Glutamatergic Neurons (Cat. No. BX-0300-30, BrainXell) using ViaFect Transfection Reagent (Cat no. E4982, Promega). Additionally, a non-targeting control, predicted not to hybridize to human transcripts, was included as a negative control. Seven days after 2′MOE treatment, total protein was extracted, cleared, and quantified as described in Example 2. Expression levels of SHANK3 were assessed following the protocols described in Example 3. FIG. 8 presents the assessment of SHANK3 protein expression in commercial iPSC-derived glutamatergic neurons (Cat. No. BX-0300-30, BrainXells) at day 7 post 2′MOE treatment (n=1 biological replicate; n=3 technical replicates). The results demonstrate that ASOs with SEQ ID NOs: 954, 1092, 1094, 1086, and 1087 increased the expression of SHANK3 protein by >=1.3-fold at 0.5 μM. Additionally, ASOs SEQ ID NOs:: 954, 1091, 1092, 1094, 1087 and 1088 showed a >=1.3-fold SHANK3 protein upregulation at 1 μM treatment in commercial iPSC-derived glutamatergic neurons (Cat. No. BX-0300-30, BrainXells). Furthermore, ASOs with SEQ ID NOs: 1085, 1089, 879, and 1084 showed a slight increased level of SHANK3 protein (>=1.1) at one or both concentrations.

### Example 6: Gymnotic Screening of Selected 2′MOE ASOs on SHANK3 Protein Levels in Human iPSC-Derived Neurons

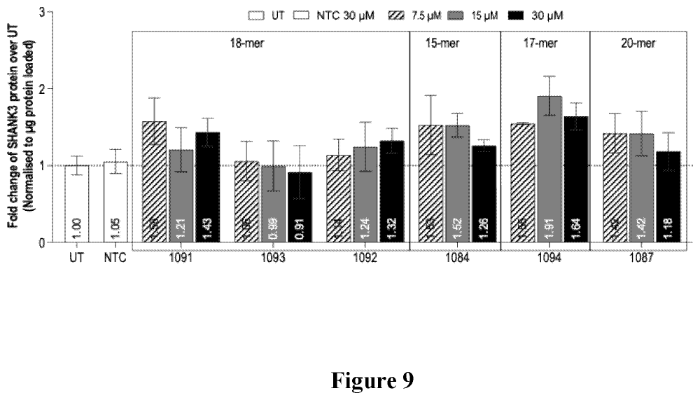

Based on the results from Example 5, six ASO sequences (SEQ ID NOs: 1084, 1087, 1091, 1092, 1093, and 1094) were selected fore screening in commercial iPSC-derived glutamatergic neurons (Cat. No. BX-0300-30, BrainXells) via gymnosis. In brief, ASOs with SEQ ID NOs: 1084, 1087, 1091, 1092, 1093, and 1094 were directly incubated with iPSC-derived glutamatergic neurons at concentrations of 7.5 μM, 15 μM, and 30 μM. At 14-day post treatment, total protein was harvested as described in Example 2 and SHANK3 protein expression levels were determined as described in Example 3. FIG. 9 illustrates the effect of the selected 2′MOEs on SHANK3 protein levels in human iPSC-derived neurons (n=1 biological replicate; n=3 technical replicates). In some test cases (SEQ ID NOs: 1091, 1084, 1094, and 1087) 2′MOE treatment demonstrated a >=1.3 fold SHANK3 protein upregulation at 7.5 μM treatment in commercial iPSC-derived glutamatergic neurons (Cat. No. BX-0300-30, BrainXells). In some other cases (SEQ ID NOs: 1084, 1094, and 1087) 2′MOE treatment demonstrated a >=1.3 fold SHANK3 protein upregulation at 15 μM treatment in commercial iPSC-derived glutamatergic neurons (Cat. No. BX-0300-30, BrainXells). In some other cases (SEQ ID NOs: 1091, 1092, and 1094) 2′MOE treatment demonstrated a >=1.3 fold SHANK3 protein upregulation at 30 μM treatment in commercial iPSC-derived glutamatergic neurons (Cat. No. BX-0300-30, BrainXells). ASOs with SEQ ID NOs: 1091, 1092, 1084, 1094, and 1087 showed a slightly increased level of SHANK3 protein (>=1.1) at one or both concentrations. The differences in protein upregulation fold change between FIG. 8 and FIG. 9 may be attributed to the transfection efficiency of each ASO, whether delivered with the transfection reagent or via gymnosis.

### Example 7: Chemical Modification of Selected MOEs to Optimize ASOs for Increased SHANK3 Protein Expression

Locked nucleic acids (LNAs) were incorporated into the selected 2′MOE ASO sequences (SEQ ID NOs: 1084, 1087, 1091, 1092, 1093, and 1094) from Example 6 in order to increase their potency and specificity. Each ASO sequence (SEQ ID NOs: 1096-1103, 1107, 1111, 1112, 1119, 1120, 1125, and 1132) was designed with the addition of two or three LNAs. In these cases, LNAs were either distributed to divide the ASO into equal parts, or placed in specific nucleotides that have mismatches to potential off-target sequences, reducing the likelihood of the ASO to hybridize in a stable manner to off-target mRNA sequences. Additionally, one or two nucleotides within the parental ASO sequences (SEQ ID NOs: 1084, 1087, 1091, 1092, 1093, and 1094) were replaced by abasic spacers. To account for the loss of binding strength when using abasic spacers, ASO sequences including abasic spacers (SEQ ID NOs: 1104-1106, 1108-1110, 1113-1118, 1121-1124, 1126-1131 and 1133) comprise one or two LNAs on the nucleotides flanking an abasic spacer. Abasic spacers were included to reduce the GC content of the parental ASO sequences (SEQ ID NOs: 1084, 1087, 1091, 1092, 1093, and 1094) to improve their tolerability and manufacturing. All ASO sequences designed with LNAs and LNAs with abasic spacers are listed in Table 3, in which LNA modified nucleotides are denoted by a “*” and abasic spacers with an “S”.

### Example 8: Effect of Selected Derivatized MOEs on SHANK3 Protein Levels in Human iPSC-Derived Neurons

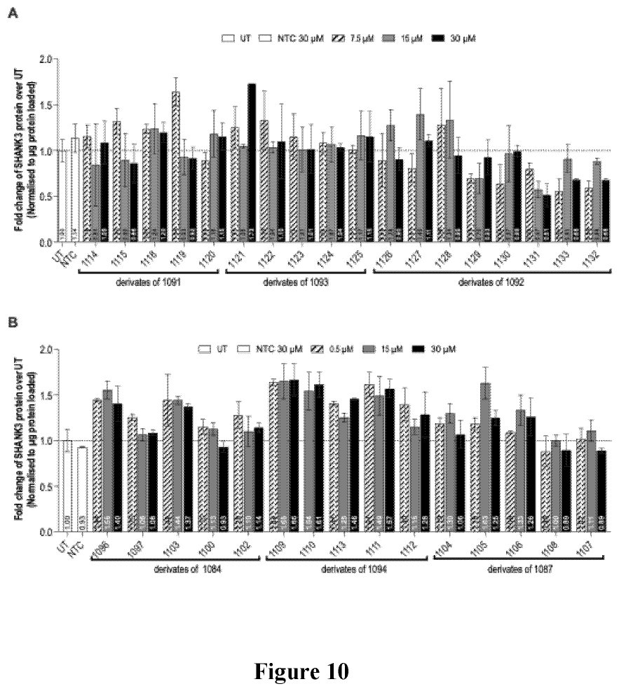

LNA modified 2′MOE ASOs (SEQ ID NOs:1096-1133) were screened in commercial iPSC-derived glutamatergic neurons (Cat. No. BX-0300-30, BrainXells) via gymnosis. ASOs were directly added to the media of seeded cells at three doses: 7.5 μM, 15 μM and 30 μM. Total protein was harvested at day 14 post-treatment as described in Example 2 and SHANK3 protein upregulation was determined as stated in Example 3. FIG. 10 illustrates the effect of LNA modified ASO sequences on SHANK3 protein levels in commercial iPSC-derived glutamatergic neurons (Cat. No. BX-0300-30, BrainXells) transfected via gymnosis (n=1 biological replicate; n=3 technical replicates). The bar plots represent the mean±SD of SHANK3 protein normalized to the total protein loaded, relative to the normalized SHANK3 protein of untreated cells. White bar plots correspond to untreated cell controls (UT) and non-targeting control (NTC); light gray, dark gray, and black bars represent cells treated with ASO at 7.5 μM, 15 μM and 30 μM, respectively. FIG. 10A shows the screening of derivates from three parental 18-mer ASO sequences (SEQ ID NOs: 1091, 1093, and 1092). In some cases, ASO sequences achieved >=1.3 fold at 7.5 μM (SEQ ID NOs: 1115, 1119, and 1122). In some other cases, ASO sequences achieved >=1.3 fold at 15 μM (SEQ ID NOs: 1127 and 1128). In some other cases, ASO sequences achieved >=1.3 fold at 30 μM (SEQ ID NO: 1121). In some other cases, ASO sequences with SEQ ID NOs: 1114, 1119, 1120, 1121, 1122, 1123, 1125, and 1127 presented a slight increase of SHANK3 protein of >=1.1-fold change. FIG. 10B shows the screening of derivates from three parental ASO sequences of diverse lengths 15-mer (SEQ ID NO:1084), 17-mer (SEQ ID NO:1094) and 20-mer (SEQ ID NO:1087). In some cases, ASO sequences achieved >=1.3 fold at 7.5 μM (SEQ ID NOs:1096, 1103, 1109, and 1111-1113). In some other cases, ASO sequences achieved 1.3 fold at 15 μM (SEQ ID NO.: 1096, 1103, 1109-1111 and 1104-1106). In some other cases, ASO sequences achieved >=1.3 fold at 30 μM (SEQ ID NOs: 1096, 1103, 1109-1111, and 1113). In some other cases, ASO sequences with SEQ ID NO: 1097, 1100, 1102, and 1107 presented a slight increase of SHANK3 protein of >=1.1-fold change.

### Example 9: Effect of Selected MOEs on SHANK3 Protein Levels in Human iPSC-Derived Neurons from Phelan-McDermid Syndrome Patients

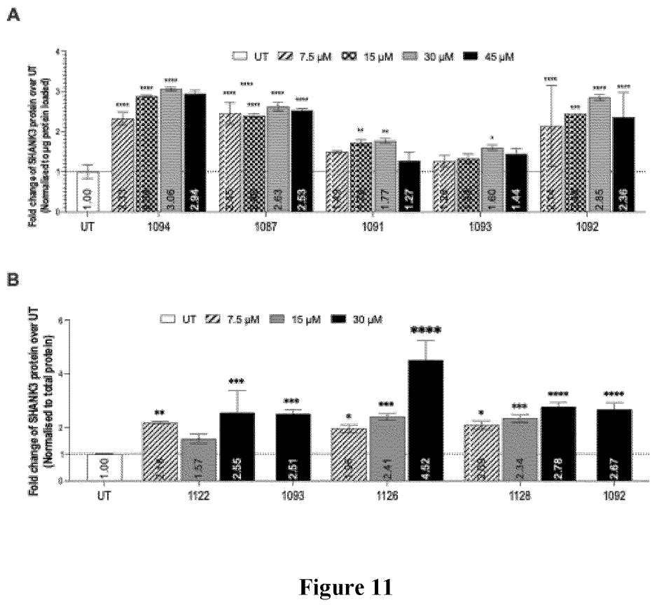

2′MOE ASOs having the nucleotide sequence corresponding to SEQ ID NOs: 1094, 1087, 1091, 1093 and 1092 were screened to test their efficacy in increasing SHANK3 protein in iPSC-derived glutamatergic neurons derived from a Phelan-McDermid Syndrome patient (PMS patient #1) via gymnotic uptake. ASOs were directly added to the media of cultured cells at four concentrations: 7.5 μM, 15 μM, 30 μM and 45 μM. Total protein was harvested at day 14 post-treatment as described in Example 2 and SHANK3 protein upregulation was determined as stated in Example 3. FIG. 11A illustrates the effect of tested ASOs on SHANK3 protein levels in iPSC-derived glutamatergic neurons derived from a Phelan-McDermid Syndrome patient transfected via gymnosis nN=1 biological replicate; n=3 technical replicates). In some cases, ASO treatment (SEQ ID NOs: 1094, 1087 and 1092) significantly increased >=1.5-fold SHANK3 protein at an ASO concentration of 7.5 μM. In some cases, ASO treatment (SEQ ID NOs: 1094, 1087, 1091 and 1092) significantly increased >=1.5-fold SHANK3 protein at an ASO concentration of 15 μM. In some cases, ASO treatment (SEQ ID NOs: 1094, 1087, 1091, 1093 and 1092) significantly increased >=1.5-fold SHANK3 protein at an ASO concentration of 30 μM. In some cases, ASO treatment (SEQ ID NOs: 1094, 1087 and 1092) significantly increased >=1.5-fold SHANK3 protein at an ASO concentration of 45 μM. In some cases, ASO treatment (SEQ ID NOs: 1094, 1087, 1091, 1093 and 1092) increased >=1.3-fold SHANK3 protein at three or all ASO concentrations tested. In some cases, ASO treatment (SEQ ID NOs: 1094, 1087, 1091, 1093 and 1092) increased >=1.1-fold SHANK3 protein at all ASO concentrations tested.

A subsequent experiment was conducted with LNA-modified and fully 2′MOE parental ASOs having nucleotide sequences corresponding to SEQ ID NOs:1122, 1093, 1126, 1128, and 1092. These ASOs were tested for their efficacy in iPSC-derived glutamatergic neurons from a Phelan-McDermid Syndrome patient (PMS patient #1) via gymnotic uptake. ASOs were incubated directly with cells at three concentrations: 7.5 μM, 15 μM and 30 μM. Parental ASO sequences with SEQ ID NOs: 1092 and 1093 were applied only at 30 μM. Total protein was harvested at day 14 post-treatment as described in Example 2, and SHANK3 protein upregulation was assessed as outlined in Example 3. FIG. 11B illustrates the effect of tested ASOs on SHANK3 protein levels in iPSC-derived glutamatergic neurons derived from a Phelan-McDermid Syndrome patient transfected via gymnosis (n=1 biological replicate; n=3 technical replicates). In all cases and concentrations tested, ASO sequences (SEQ ID NOs: 1122, 1093, 1126, 1128, and 1092) achieved >=1.5-fold change of SHANK3 protein upregulation over untreated cells. Significance of the fold change was calculated with a one-way ANOVA test where (*) denotes a p. adj <0.05, (**) a p. adj <0.01, (***) a p. adj <0.005 and (****) a p. adj <0.001.

### Example 10: Effect of Selected MOEs on SHANK3 Protein Levels in Human iPSC-Derived Neurons from Phelan-McDermid Syndrome Patients

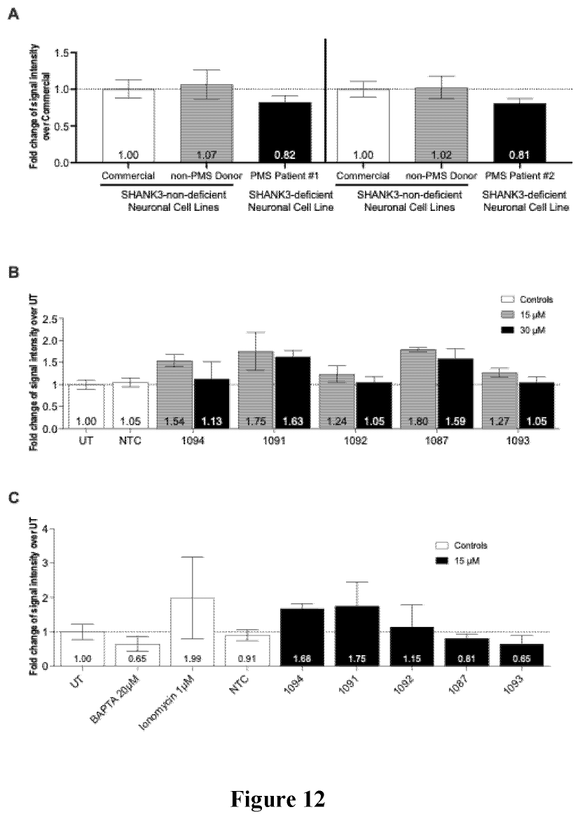

Spontaneous calcium oscillations have been shown to be impaired in PMS patient-derived neurons compared to unaffected control neurons (Darville, et al. 2016). To investigated whether PMS patient iPSC-derived neurons in this study show similar impairments in neuronal function, calcium oscillation activity was assessed in two independent PMS patient lines (PMS patient #1 and #2) compared to two non-PMS (non-SHANK3-deficient) control lines. In brief, intracellular calcium was measured with Fluo-4 Direct™ (cat no. F10471, ThermoFisher) following manufacture's protocol, and plates were incubated at 37° C. for 60 min. Plates were read on CLARIOstar (software version 5.70 R3) with fluorescence mode at 494/516 nm (ex/em) for 1 minute at 0.1 sec intervals. Raw data was pre-processed in R program adapted from Per Uhlen, 2004 for Spectral Analysis of Calcium Oscillations, in which a trend correction and catering of the signal is performed followed by normalizing the data to frequency to obtain the power spectral density, referred as signal intensity. Signal intensities lower than the mean signal intensity from blanks (wells without cells) were filter out. Remaining data was used to calculate the median signal intensity for each sample and condition. FIG. 12A illustrates the signal intensity baseline of the calcium oscillations in two PMS patient and two non-PMS control iPSC-derived glutamatergic neurons (commercial BrainXells and donor iPSC). The bar plots are divided in two sets, one for each PMS cell line, as PMS patient #1 together with its non-PMS controls was seeded at 25,000-30,000 cells per well, while the set of PMS patient #2 at 10,000 cells per well. Both PMS patient lines show a decreased signal intensity compared to both SHANK3-non-deficient lines. To test whether increased SHANK3 protein following ASO treatment resulted in increased neuronal activity in Phelan-McDermid patient iPSC-derived glutamatergic neurons (PMS patient #1), calcium oscillations were assessed 21 days post gymnotic ASO treatment. In brief, iPSC-derived neurons were single-dose treated with 2′MOEs corresponding to SEQ ID NOs: 1094, 1091, 1092, 1087, and 1093 at 15 μM and 30 μM and assessed 21 days post-treatment (FIG. 12B). Additionally, a double-dose treatment with SEQ ID NOs:1094, 1091, 1092, 1087 and 1093 was performed for the 15 μM, 7 days after the first treatment (FIG. 12C). At day 21 post-treatment, intracellular calcium was measured and analysed as described above. Untreated ASO wells were incubated for 60 minutes with BAPTA-AM, as a negative control, at 20 μM (Cat no. B1205, Invitrogen); or positive control Ionomycin at 1 μM (Cat no. 124222, Invitrogen) prior to plate reading (FIG. 12C). FIG. 12B illustrates the change in the signal intensity of the calcium oscillations as a response to the single-dose gymnotic ASO treatment at 15 μM and 30 μM, day-21 post treatment. All ASO treatments with SEQ ID NO: 1094, 1091, 1092, 1087 and 1093 increased (>=1.1-fold change) calcium oscillations signal intensity compared to PMS untreated cells. FIG. 12C illustrates the change in the signal intensity of the calcium oscillations as a response to the double-dose gymnotic ASO treatment at 15 μM on day-21 post treatment. ASO treatment with SEQ ID NO: 1094, 1091 and 1092 increased (>=1.1-fold change) the signal intensity of calcium oscillations compared to PMS untreated cells.

### Example 11: Identification of SHANK3 Target Sequences

Identification of annotated and expressed transcripts that could generate the canonical transcript of the SHANK3 gene was performed by the sequence alignment of all SHANK3 protein-coding and NMD transcripts described in Gencode v38. An ASO sequence “micro-walk” of 18mers and 25-mers in 1 bp increments was performed over the sequences of intron 7, 17, and 21 of the ENST00000262795.6 pre-mRNA transcript and designed to target the intronic splice enhancer motif to mediate exclusion of the retained intron or part thereof and generate productive SHANK3 mRNA transcript. The resulting ASO sequences correspond to SEQ ID NOs:4584-11700 provided in the accompanying sequence listing (incorporated by reference herein).

It will be appreciated by persons skilled in the art that numerous variations and/or modifications may be made to the invention as shown in the specific examples without departing from the spirit or scope of the invention as broadly described. The present examples are, therefore, to be considered in all respects as illustrative and not restrictive.

This application claims priority from: Australian Provisional Application No. 2023903841 filed on 29 Nov. 2023; Australian Provisional Application No. 2023903842 filed on 29 Nov. 2023; Australian Provisional Application No. 2023903916 filed on 4 Dec. 2023; Australian Provisional Application No. 2023903924 filed on 5 Dec. 2023; Australian Provisional Application No. 2023903927 filed on 6 Jun. 2024; Australian Provisional Application No. 2024901708 filed on 6 Jun. 2024, Australian Provisional Application No. 2024902223 filed on 17 Jul. 2024; and Australian Provisional Application No. 2024903714 filed on 13 Nov. 2024, the entire contents of each of which are hereby incorporated by reference in their entirety.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like that have been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Alia et al., (2019), Frontiers in Neuroscience, 13:684.
Boyé et al., (2022), Nature Communications, 13:1169.doi.org/10.1038/s41467-022-28785-9.
Darville et al., (2016), eBioMedicine, 9:293-305.
Dugal-Tessier et al., (2021), J Clin Med., 10(4):838.
Fitzgerald et al., (2020), Stem Cells, 38(11):1375-1386.
Kim et al., (2014), Frontiers in Neuroscience, 8:109.
Monteiro et al. (2017), Nature Rev Neurosci, 18(3):147-157.
Prakash et al., (2019), Nucleic Acids Research, 47(12):6029-6044.
Rak et al., (2016), J Gene Med, 18(11-12):331-342.

APPENDIX
Sequences and SEQ ID NOS

SEQ ID NO: 1
SHANK3 mRNA Canonical Transcript 3′ UTR Sequence (from SHANK3-
201 ENST00000262795.7)
CGCCCCACCCCCACTCCCGCCCCGGCCGTGCCCTGCCGGCAGGGCCCCCCACCCCCACCC
CGGGCCGCGGGCTCGGCCTGCCCCTTACGACGGCGCCCGGGCCAGGAATGTTGCATGAAT
CGTCCTGTTTGCTGTTGCTCGGAGACTCGCCCTGTACATTGCTTAGTGCCCTCACCGGCC
GCCCAGCCCACCCAGCGCACAGTCAGGAAGGGCGTGGACCAGGGAGGCTGGGGGGGGAGG
TGCCGGGGGTGGGGTGCCCTAGCGTGACCACCTCCTTCGCAGCTCCTGGTGGCCATTCTC
CCAGAGGGGGAACCTAGTCCAGCATGCGAGGTCAGGACCCGCCTTGGTGACTCGGGGGGA
GGGGGGAGACATTGGGATTCTCGATGGGGGCCAAGGAGCCCCCCTGTTTTGCATATTTTA
ATCCACTCTATATTTGGAACGAGAAAAGGAACAAATATCTCTGTCCGTAATAGTTTCCTC
TCCCCTCCCTTCTACTTCCACTGGTCCCACTGCAGCTGCCCAGTCTTCCATCTCCGGCCC
CTCACTGCCACTGCCACCCCACAACGGGGCAGGGGACGCTCCAGCTGGTCTGGGGTTGGC
CAGGGCCCTAGTGGCCCGCCCTGGGGCCCCAGCTCGGCCCCTCGCCTCGCTGAGCTCTAG
TGTGCCCCACCGACCCTTCAGGTGCTGCTCGTGGTGGGAGGGGCGGCAGGCCGCGGGTCC
TGCTGTGCACCCGCGGGACCAGCCGGCCTGGGAGACCATCGGCCGGGGGGGATGAGGGCA
GGGCCCTGCCGCTCCACCGCAGCCATCTTCCTCACAGGGTCTCTCCCCAAGGAGGGGGCT
AGCTTGGTCCCCATGCTCTTGGGCAACTACAGCAGAGAAGCCTCCCTGCCTTGGACCCCA
AAGTCTCCTGTCCTGCCCTTTATGTGTGTGGGTGAAACTGGGTGCGTCTGAGCACGTGGG
AGCCGTGTGTGTGCCTGATTACTGAGTGGCCACCAGGGGCCGCTCTGGACTAGCGCGGGG
CCGTGGAGGCGTGCACCGTGTGCATGCGTGGGGTGTACCTGTGAGAGCACCCTGTCTCCT
CTTCCAAAGAAAGTCAGAGGCCATCCTGCACCCTGGGTCCAGCTGTTTGCCCAGCCTGTC
CTTCCAGAGCCTCACCCAGCCTGAGCGGGGTTCCCTGGTGAATCCCTGCTGCTTGGGGAG
GCCCCAAGGGCCCCTTGGAGGCAGCGCCCCCACCTTGGGCTTCTGAGGGCATCATAGGGG
GACCCCTAGAGTCAGTTCACCACAGGCCCTGGGGAGAGTCAAAGACCCCCGAGGGTGCCC
AGCCCCCCACACTGTGACTCCTCACACTCAGCGATGACCTGTGGGGTGGGGGGCCCTGGG
ACGTTTTTAAACCTAGGGTTTGGAGTCTGGACTAAGCTCCATCCACGTCACTCACAAGTT
TCTGTTTATATTTCTAGCTTTTTTTAATAAAATAAAAAAAAAAAGAAAACAGAAGTTTTC
ACAACCCAGGGGCCTGGCACGCCGGTCTGTGCCTGCCCGCCCCGCCCTGGCCCACCGGCC
CCACTCCCTGGGCACAGAGTCACACCCACTCATCCTTCCGCCAACAGTCCAGGTCACACA
GCAGCAGTCACTGTAACAGACTGCCACATACACACTCGGTCTCACACTCACCTGTGGGTT
TTGGTTCCGTTCAATTTGGGTTTTTAACTTTACAGGGTCAGTTCCGCTTCACCTCCTTTT
GTATGGAGTTCCATCCGGGGGGTTTCACCCCCTGCTCCAGTCCTGAGGCCTCCTGACCCT
GACGTTGTGATACGCCCCACAGAGATCTATGTTTCTTATATTATTATTATTGATAATAAT
TATTATAATATTATTATGTAATAAATTTATAAGAAATGAA
SEQ ID NO: 2
CPP Amino Acid Sequence (Artificial/Synthetic)
RRSRTARAGRPGRNSSRPSAPR
SEQ ID NO: 1079
target region within the 3′UTR of SHANK3
TTTTGGTTCCGTTCAATTTGGGTTTTTAACTTTACAGGGTCAGTTCCGCTTCACCTCCTTTT
GTATGGAGTTCCAT

TABLE 1
Exemplary ASO or AR Sequences Targeting
SHANK3 mRNA 3′ UTR

| SEQ | | | |
|---|---|---|---|
| ID | | | ASO |
| NO | Sequence | Name | type |
| 3 | GTAAAGTTAAAAACCCAAATTGAAC | SHANK3 H22A(+1689+1713) | 25 mer |
| 4 | TTAAAGTTAAAAACCCAAATTGAAC | SHANK3 H22A(+1689+1713) 1mm_1 | 25 mer |
| 5 | GTAAAGTTAAAAATCCAAATTGAAC | SHANK3 H22A(+1689+1713) 1mm_14 | 25 mer |
| 6 | GTAAAGTTAAAAACTCAAATTGAAC | SHANK3 H22A(+1689+1713) 1mm_15 | 25 mer |
| 7 | GTAAAGTTAAAAACCTAAATTGAAC | SHANK3 H22A(+1689+1713) 1mm_16 | 25 mer |
| 8 | GTAAAGTTAAAAACCCAAATTTAAC | SHANK3 H22A(+1689+1713) 1mm_22 | 25 mer |
| 9 | GTAAAGTTAAAAACCCAAATTGAAT | SHANK3 H22A(+1689+1713) 1mm_25 | 25 mer |
| 10 | GTAAATTTAAAAACCCAAATTGAAC | SHANK3 H22A(+1689+1713) 1mm_6 | 25 mer |
| 11 | ATAAAATTAAAAACCCAAATTGAAC | SHANK3 H22A(+1689+1713) 2mm_1_6 | 25 mer |
| 12 | GTAAAGTTAAAAACCCAAATTAAAC | SHANK3 H22A(+1689+1713) mm1 | 25 mer |
| 13 | GTAAAATTAAAAACCCAAATTAAAC | SHANK3 H22A(+1689+1713) mm2 | 25 mer |
| 14 | TGTAAAGTTAAAAATCCAAATTGAA | SHANK3 H22A(+1690+1714) 1mm_15 | 25 mer |
| 15 | TGTAAAGTTAAAAACTCAAATTGAA | SHANK3 H22A(+1690+1714) 1mm_16 | 25 mer |
| 16 | TGTAAAGTTAAAAACCTAAATTGAA | SHANK3 H22A(+1690+1714) 1mm_17 | 25 mer |
| 17 | TTTAAAGTTAAAAACCCAAATTGAA | SHANK3 H22A(+1690+1714) 1mm_2 | 25 mer |
| 18 | TGTAAAGTTAAAAACCCAAATTTAA | SHANK3 H22A(+1690+1714) 1mm_23 | 25 mer |
| 19 | TGTAAATTTAAAAACCCAAATTGAA | SHANK3 H22A(+1690+1714) 1mm_7 | 25 mer |
| 20 | TGTAAAGTTAAAAACCCAAATTAAA | SHANK3 H22A(+1690+1714) mm1 | 25 mer |
| 21 | TGTAAAATTAAAAACCCAAATTAAA | SHANK3 H22A(+1690+1714) mm2 | 25 mer |
| 22 | CTGTAAAGTTAAAAACCCAAATTGA | SHANK3 H22A(+1691+1715) | 25 mer |
| 23 | TTGTAAAGTTAAAAACCCAAATTGA | SHANK3 H22A(+1691+1715) 1mm_1 | 25 mer |
| 24 | CTGTAAAGTTAAAAATCCAAATTGA | SHANK3 H22A(+1691+1715) 1mm_16 | 25 mer |
| 25 | CTGTAAAGTTAAAAACTCAAATTGA | SHANK3 H22A(+1691+1715) 1mm_17 | 25 mer |
| 26 | CTGTAAAGTTAAAAACCTAAATTGA | SHANK3 H22A(+1691+1715) 1mm_18 | 25 mer |
| 27 | CTGTAAAGTTAAAAACCCAAATTTA | SHANK3 H22A(+1691+1715) 1mm_24 | 25 mer |
| 28 | CTTTAAAGTTAAAAACCCAAATTGA | SHANK3 H22A(+1691+1715) 1mm_3 | 25 mer |
| 29 | CTGTAAATTTAAAAACCCAAATTGA | SHANK3 H22A(+1691+1715) 1mm_8 | 25 mer |
| 30 | CTGTAAAGTTAAAAACCCAAATTAA | SHANK3 H22A(+1691+1715) mm1 | 25 mer |
| 31 | CTGTAAAATTAAAAACCCAAATTAA | SHANK3 H22A(+1691+1715) mm2 | 25 mer |
| 32 | TCTGTAAAGTTAAAAACCCAAATTG | SHANK3 H22A(+1692+1716) 1mm_1 | 25 mer |
| 33 | CCTGTAAAGTTAAAAATCCAAATTG | SHANK3 H22A(+1692+1716) 1mm_17 | 25 mer |
| 34 | CCTGTAAAGTTAAAAACTCAAATTG | SHANK3 H22A(+1692+1716) 1mm_18 | 25 mer |
| 35 | CCTGTAAAGTTAAAAACCTAAATTG | SHANK3 H22A(+1692+1716) 1mm_19 | 25 mer |
| 36 | CTTGTAAAGTTAAAAACCCAAATTG | SHANK3 H22A(+1692+1716) 1mm_2 | 25 mer |
| 37 | CCTTTAAAGTTAAAAACCCAAATTG | SHANK3 H22A(+1692+1716) 1mm_4 | 25 mer |
| 38 | CCTGTAAATTTAAAAACCCAAATTG | SHANK3 H22A(+1692+1716) 1mm_9 | 25 mer |
| 39 | CCTGTAAAGTTAAAAACCCAAATTA | SHANK3 H22A(+1692+1716) mm1 | 25 mer |
| 40 | CCTGTAAAATTAAAAACCCAAATTA | SHANK3 H22A(+1692+1716) mm2 | 25 mer |
| 41 | TCCTGTAAAGTTAAAAACCCAAATT | SHANK3 H22A(+1693+1717) 1mm_1 | 25 mer |
| 42 | CCCTGTAAATTTAAAAACCCAAATT | SHANK3 H22A(+1693+1717) 1mm_10 | 25 mer |
| 43 | CCCTGTAAAGTTAAAAATCCAAATT | SHANK3 H22A(+1693+1717) 1mm_18 | 25 mer |
| 44 | CCCTGTAAAGTTAAAAACTCAAATT | SHANK3 H22A(+1693+1717) 1mm_19 | 25 mer |
| 45 | CTCTGTAAAGTTAAAAACCCAAATT | SHANK3 H22A(+1693+1717) 1mm_2 | 25 mer |
| 46 | CCCTGTAAAGTTAAAAACCTAAATT | SHANK3 H22A(+1693+1717) 1mm_20 | 25 mer |
| 47 | CCTTGTAAAGTTAAAAACCCAAATT | SHANK3 H22A(+1693+1717) 1mm_3 | 25 mer |
| 48 | CCCTTTAAAGTTAAAAACCCAAATT | SHANK3 H22A(+1693+1717) 1mm_5 | 25 mer |
| 49 | CCCTGTAAAATTAAAAACCCAAATT | SHANK3 H22A(+1693+1717) mm1 | 25 mer |
| 50 | CCCTATAAAATTAAAAACCCAAATT | SHANK3 H22A(+1693+1717) mm2 | 25 mer |
| 51 | ACCCTGTAAAGTTAAAAACCCAAAT | SHANK3 H22A(+1694+1718) | 25 mer |
| 52 | ACCCTGTAAATTTAAAAACCCAAAT | SHANK3 H22A(+1694+1718) 1mm_11 | 25 mer |
| 53 | ACCCTGTAAAGTTAAAAATCCAAAT | SHANK3 H22A(+1694+1718) 1mm_19 | 25 mer |
| 54 | ATCCTGTAAAGTTAAAAACCCAAAT | SHANK3 H22A(+1694+1718) 1mm_2 | 25 mer |
| 55 | ACCCTGTAAAGTTAAAAACTCAAAT | SHANK3 H22A(+1694+1718) 1mm_20 | 25 mer |
| 56 | ACCCTGTAAAGTTAAAAACCTAAAT | SHANK3 H22A(+1694+1718) 1mm_21 | 25 mer |
| 57 | ACTCTGTAAAGTTAAAAACCCAAAT | SHANK3 H22A(+1694+1718) 1mm_3 | 25 mer |
| 58 | ACCTTGTAAAGTTAAAAACCCAAAT | SHANK3 H22A(+1694+1718) 1mm_4 | 25 mer |
| 59 | ACCCTTTAAAGTTAAAAACCCAAAT | SHANK3 H22A(+1694+1718) 1mm_6 | 25 mer |
| 60 | ACCCTGTAAAATTAAAAACCCAAAT | SHANK3 H22A(+1694+1718) mm1 | 25 mer |
| 61 | ACCCTATAAAATTAAAAACCCAAAT | SHANK3 H22A(+1694+1718) mm2 | 25 mer |
| 62 | GACCCTGTAAAGTTAAAAACCCAAA | SHANK3 H22A(+1695+1719) | 25 mer |
| 63 | TACCCTGTAAAGTTAAAAACCCAAA | SHANK3 H22A(+1695+1719) 1mm_1 | 25 mer |
| 64 | GACCCTGTAAATTTAAAAACCCAAA | SHANK3 H22A(+1695+1719) 1mm_12 | 25 mer |
| 65 | GACCCTGTAAAGTTAAAAATCCAAA | SHANK3 H22A(+1695+1719) 1mm_20 | 25 mer |
| 66 | GACCCTGTAAAGTTAAAAACTCAAA | SHANK3 H22A(+1695+1719) 1mm_21 | 25 mer |
| 67 | GACCCTGTAAAGTTAAAAACCTAAA | SHANK3 H22A(+1695+1719) 1mm_22 | 25 mer |
| 68 | GATCCTGTAAAGTTAAAAACCCAAA | SHANK3 H22A(+1695+1719) 1mm_3 | 25 mer |
| 69 | GACTCTGTAAAGTTAAAAACCCAAA | SHANK3 H22A(+1695+1719) 1mm_4 | 25 mer |
| 70 | GACCTTGTAAAGTTAAAAACCCAAA | SHANK3 H22A(+1695+1719) 1mm_5 | 25 mer |
| 71 | GACCCTTTAAAGTTAAAAACCCAAA | SHANK3 H22A(+1695+1719) 1mm_7 | 25 mer |
| 72 | GACCCTGTAAAATTAAAAACCCAAA | SHANK3 H22A(+1695+1719) mm1 | 25 mer |
| 73 | GACCCTATAAAATTAAAAACCCAAA | SHANK3 H22A(+1695+1719) mm2 | 25 mer |
| 74 | TGACCCTGTAAATTTAAAAACCCAA | SHANK3 H22A(+1696+1720) 1mm_13 | 25 mer |
| 75 | TTACCCTGTAAAGTTAAAAACCCAA | SHANK3 H22A(+1696+1720) 1mm_2 | 25 mer |
| 76 | TGACCCTGTAAAGTTAAAAATCCAA | SHANK3 H22A(+1696+1720) 1mm_21 | 25 mer |
| 77 | TGACCCTGTAAAGTTAAAAACTCAA | SHANK3 H22A(+1696+1720) 1mm_22 | 25 mer |
| 78 | TGACCCTGTAAAGTTAAAAACCTAA | SHANK3 H22A(+1696+1720) 1mm_23 | 25 mer |
| 79 | TGATCCTGTAAAGTTAAAAACCCAA | SHANK3 H22A(+1696+1720) 1mm_4 | 25 mer |
| 80 | TGACTCTGTAAAGTTAAAAACCCAA | SHANK3 H22A(+1696+1720) 1mm_5 | 25 mer |
| 81 | TGACCTTGTAAAGTTAAAAACCCAA | SHANK3 H22A(+1696+1720) 1mm_6 | 25 mer |
| 82 | TGACCCTTTAAAGTTAAAAACCCAA | SHANK3 H22A(+1696+1720) 1mm_8 | 25 mer |
| 83 | TAACCCTGTAAAATTAAAAACCCAA | SHANK3 H22A(+1696+1720) 2mm_2_13 | 25 mer |
| 84 | TGACCCTGTAAAATTAAAAACCCAA | SHANK3 H22A(+1696+1720) mm1 | 25 mer |
| 85 | TGACCCTATAAAATTAAAAACCCAA | SHANK3 H22A(+1696+1720) mm2 | 25 mer |
| 86 | CTGACCCTGTAAAGTTAAAAACCCA | SHANK3 H22A(+1697+1721) | 25 mer |
| 87 | TTGACCCTGTAAAGTTAAAAACCCA | SHANK3 H22A(+1697+1721) 1mm_1 | 25 mer |
| 88 | CTGACCCTGTAAATTTAAAAACCCA | SHANK3 H22A(+1697+1721) 1mm_14 | 25 mer |
| 89 | CTGACCCTGTAAAGTTAAAAATCCA | SHANK3 H22A(+1697+1721) 1mm_22 | 25 mer |
| 90 | CTGACCCTGTAAAGTTAAAAACTCA | SHANK3 H22A(+1697+1721) 1mm_23 | 25 mer |
| 91 | CTGACCCTGTAAAGTTAAAAACCTA | SHANK3 H22A(+1697+1721) 1mm_24 | 25 mer |
| 92 | CTTACCCTGTAAAGTTAAAAACCCA | SHANK3 H22A(+1697+1721) 1mm_3 | 25 mer |
| 93 | CTGATCCTGTAAAGTTAAAAACCCA | SHANK3 H22A(+1697+1721) 1mm_5 | 25 mer |
| 94 | CTGACTCTGTAAAGTTAAAAACCCA | SHANK3 H22A(+1697+1721) 1mm_6 | 25 mer |
| 95 | CTGACCTTGTAAAGTTAAAAACCCA | SHANK3 H22A(+1697+1721) 1mm_7 | 25 mer |
| 96 | CTGACCCTTTAAAGTTAAAAACCCA | SHANK3 H22A(+1697+1721) 1mm_9 | 25 mer |
| 97 | CTAACCCTGTAAAATTAAAAACCCA | SHANK3 H22A(+1697+1721) 2mm_3_14 | 25 mer |
| 98 | CTGACCCTGTAAAATTAAAAACCCA | SHANK3 H22A(+1697+1721) mm1 | 25 mer |
| 99 | CTGACCCTATAAAATTAAAAACCCA | SHANK3 H22A(+1697+1721) mm2 | 25 mer |
| 100 | ACTGACCCTGTAAAGTTAAAAACCC | SHANK3 H22A(+1698+1722) | 25 mer |
| 101 | ACTGACCCTTTAAAGTTAAAAACCC | SHANK3 H22A(+1698+1722) 1mm_10 | 25 mer |
| 102 | ACTGACCCTGTAAATTTAAAAACCC | SHANK3 H22A(+1698+1722) 1mm_15 | 25 mer |
| 103 | ATTGACCCTGTAAAGTTAAAAACCC | SHANK3 H22A(+1698+1722) 1mm_2 | 25 mer |
| 104 | ACTGACCCTGTAAAGTTAAAAATCC | SHANK3 H22A(+1698+1722) 1mm_23 | 25 mer |
| 105 | ACTGACCCTGTAAAGTTAAAAACTC | SHANK3 H22A(+1698+1722) 1mm_24 | 25 mer |
| 106 | ACTGACCCTGTAAAGTTAAAAACCT | SHANK3 H22A(+1698+1722) 1mm_25 | 25 mer |
| 107 | ACTTACCCTGTAAAGTTAAAAACCC | SHANK3 H22A(+1698+1722) 1mm_4 | 25 mer |
| 108 | ACTGATCCTGTAAAGTTAAAAACCC | SHANK3 H22A(+1698+1722) 1mm_6 | 25 mer |
| 109 | ACTGACTCTGTAAAGTTAAAAACCC | SHANK3 H22A(+1698+1722) 1mm_7 | 25 mer |
| 110 | ACTGACCTTGTAAAGTTAAAAACCC | SHANK3 H22A(+1698+1722) 1mm_8 | 25 mer |
| 111 | ACTAACCCTGTAAAATTAAAAACCC | SHANK3 H22A(+1698+1722) 2mm_4_15 | 25 mer |
| 112 | ACTGACCCTGTAAAATTAAAAACCC | SHANK3 H22A(+1698+1722) mm1 | 25 mer |
| 113 | ACTGACCCTATAAAATTAAAAACCC | SHANK3 H22A(+1698+1722) mm2 | 25 mer |
| 114 | AACTGACCCTTTAAAGTTAAAAACC | SHANK3 H22A(+1699+1723) 1mm_11 | 25 mer |
| 115 | AACTGACCCTGTAAATTTAAAAACC | SHANK3 H22A(+1699+1723) 1mm_16 | 25 mer |
| 116 | AACTGACCCTGTAAAGTTAAAAATC | SHANK3 H22A(+1699+1723) 1mm_24 | 25 mer |
| 117 | AACTGACCCTGTAAAGTTAAAAACT | SHANK3 H22A(+1699+1723) 1mm_25 | 25 mer |
| 118 | AATTGACCCTGTAAAGTTAAAAACC | SHANK3 H22A(+1699+1723) 1mm_3 | 25 mer |
| 119 | AACTTACCCTGTAAAGTTAAAAACC | SHANK3 H22A(+1699+1723) 1mm_5 | 25 mer |
| 120 | AACTGATCCTGTAAAGTTAAAAACC | SHANK3 H22A(+1699+1723) 1mm_7 | 25 mer |
| 121 | AACTGACTCTGTAAAGTTAAAAACC | SHANK3 H22A(+1699+1723) 1mm_8 | 25 mer |
| 122 | AACTGACCTTGTAAAGTTAAAAACC | SHANK3 H22A(+1699+1723) 1mm_9 | 25 mer |
| 123 | AACTAACCCTGTAAAATTAAAAACC | SHANK3 H22A(+1699+1723) 2mm_5_16 | 25 mer |
| 124 | AACTGACCCTGTAAAATTAAAAACC | SHANK3 H22A(+1699+1723) mm1 | 25 mer |
| 125 | AACTGACCCTATAAAATTAAAAACC | SHANK3 H22A(+1699+1723) mm2 | 25 mer |
| 126 | GAACTGACCCTGTAAAGTTAAAAAC | SHANK3 H22A(+1700+1724) | 25 mer |
| 127 | TAACTGACCCTGTAAAGTTAAAAAC | SHANK3 H22A(+1700+1724) 1mm_1 | 25 mer |
| 128 | GAACTGACCTTGTAAAGTTAAAAAC | SHANK3 H22A(+1700+1724) 1mm_10 | 25 mer |
| 129 | GAACTGACCCTTTAAAGTTAAAAAC | SHANK3 H22A(+1700+1724) 1mm_12 | 25 mer |
| 130 | GAACTGACCCTGTAAATTTAAAAAC | SHANK3 H22A(+1700+1724) 1mm_17 | 25 mer |
| 131 | GAACTGACCCTGTAAAGTTAAAAAT | SHANK3 H22A(+1700+1724) 1mm_25 | 25 mer |
| 132 | GAATTGACCCTGTAAAGTTAAAAAC | SHANK3 H22A(+1700+1724) 1mm_4 | 25 mer |
| 133 | GAACTTACCCTGTAAAGTTAAAAAC | SHANK3 H22A(+1700+1724) 1mm_6 | 25 mer |
| 134 | GAACTGATCCTGTAAAGTTAAAAAC | SHANK3 H22A(+1700+1724) 1mm_8 | 25 mer |
| 135 | GAACTGACTCTGTAAAGTTAAAAAC | SHANK3 H22A(+1700+1724) 1mm_9 | 25 mer |
| 136 | AAACTGACCCTATAAAGTTAAAAAC | SHANK3 H22A(+1700+1724) 2mm_1_12 | 25 mer |
| 137 | AAACTGACCCTGTAAAATTAAAAAC | SHANK3 H22A(+1700+1724) 2mm_1_17 | 25 mer |
| 138 | AAACTAACCCTGTAAAGTTAAAAAC | SHANK3 H22A(+1700+1724) 2mm_1_6 | 25 mer |
| 139 | GAACTAACCCTGTAAAATTAAAAAC | SHANK3 H22A(+1700+1724) 2mm_6_17 | 25 mer |
| 140 | GAACTGACCCTGTAAAATTAAAAAC | SHANK3 H22A(+1700+1724) mm1 | 25 mer |
| 141 | GAACTGACCCTATAAAATTAAAAAC | SHANK3 H22A(+1700+1724) mm2 | 25 mer |
| 142 | GGAACTGACCCTGTAAAGTTAAAAA | SHANK3 H22A(+1701+1725) | 25 mer |
| 143 | TGAACTGACCCTGTAAAGTTAAAAA | SHANK3 H22A(+1701+1725) 1mm_1 | 25 mer |
| 144 | GGAACTGACTCTGTAAAGTTAAAAA | SHANK3 H22A(+1701+1725) 1mm_10 | 25 mer |
| 145 | GGAACTGACCTTGTAAAGTTAAAAA | SHANK3 H22A(+1701+1725) 1mm_11 | 25 mer |
| 146 | GGAACTGACCCTTTAAAGTTAAAAA | SHANK3 H22A(+1701+1725) 1mm_13 | 25 mer |
| 147 | GGAACTGACCCTGTAAATTTAAAAA | SHANK3 H22A(+1701+1725) 1mm_18 | 25 mer |
| 148 | GTAACTGACCCTGTAAAGTTAAAAA | SHANK3 H22A(+1701+1725) 1mm_2 | 25 mer |
| 149 | GGAATTGACCCTGTAAAGTTAAAAA | SHANK3 H22A(+1701+1725) 1mm_5 | 25 mer |
| 150 | GGAACTTACCCTGTAAAGTTAAAAA | SHANK3 H22A(+1701+1725) 1mm_7 | 25 mer |
| 151 | GGAACTGATCCTGTAAAGTTAAAAA | SHANK3 H22A(+1701+1725) 1mm_9 | 25 mer |
| 152 | AGAACTGACCCTATAAAGTTAAAAA | SHANK3 H22A(+1701+1725) 2mm_1_13 | 25 mer |
| 153 | AGAACTGACCCTGTAAAATTAAAAA | SHANK3 H22A(+1701+1725) 2mm_1_18 | 25 mer |
| 154 | AAAACTGACCCTGTAAAGTTAAAAA | SHANK3 H22A(+1701+1725) 2mm_1_2 | 25 mer |
| 155 | AGAACTAACCCTGTAAAGTTAAAAA | SHANK3 H22A(+1701+1725) 2mm_1_7 | 25 mer |
| 156 | GAAACTGACCCTATAAAGTTAAAAA | SHANK3 H22A(+1701+1725) 2mm_2_13 | 25 mer |
| 157 | GAAACTGACCCTGTAAAATTAAAAA | SHANK3 H22A(+1701+1725) 2mm_2_18 | 25 mer |
| 158 | GAAACTAACCCTGTAAAGTTAAAAA | SHANK3 H22A(+1701+1725) 2mm_2_7 | 25 mer |
| 159 | GGAACTAACCCTGTAAAATTAAAAA | SHANK3 H22A(+1701+1725) 2mm_7_18 | 25 mer |
| 160 | GGAACTGACCCTGTAAAATTAAAAA | SHANK3 H22A(+1701+1725) mm1 | 25 mer |
| 161 | GGAACTGACCCTATAAAATTAAAAA | SHANK3 H22A(+1701+1725) mm2 | 25 mer |
| 162 | TGGAACTGACCCTGTAAAGTTAAAA | SHANK3 H22A(+1702+1726) 1mm_1 | 25 mer |
| 163 | CGGAACTGATCCTGTAAAGTTAAAA | SHANK3 H22A(+1702+1726) 1mm_10 | 25 mer |
| 164 | CGGAACTGACTCTGTAAAGTTAAAA | SHANK3 H22A(+1702+1726) 1mm_11 | 25 mer |
| 165 | CGGAACTGACCTTGTAAAGTTAAAA | SHANK3 H22A(+1702+1726) 1mm_12 | 25 mer |
| 166 | CGGAACTGACCCTTTAAAGTTAAAA | SHANK3 H22A(+1702+1726) 1mm_14 | 25 mer |
| 167 | CGGAACTGACCCTGTAAATTTAAAA | SHANK3 H22A(+1702+1726) 1mm_19 | 25 mer |
| 168 | CTGAACTGACCCTGTAAAGTTAAAA | SHANK3 H22A(+1702+1726) 1mm_2 | 25 mer |
| 169 | CGTAACTGACCCTGTAAAGTTAAAA | SHANK3 H22A(+1702+1726) 1mm_3 | 25 mer |
| 170 | CGGAATTGACCCTGTAAAGTTAAAA | SHANK3 H22A(+1702+1726) 1mm_6 | 25 mer |
| 171 | CGGAACTTACCCTGTAAAGTTAAAA | SHANK3 H22A(+1702+1726) 1mm_8 | 25 mer |
| 172 | CAGAACTGACCCTATAAAGTTAAAA | SHANK3 H22A(+1702+1726) 2mm_2_14 | 25 mer |
| 173 | CAGAACTGACCCTGTAAAATTAAAA | SHANK3 H22A(+1702+1726) 2mm_2_19 | 25 mer |
| 174 | CAAAACTGACCCTGTAAAGTTAAAA | SHANK3 H22A(+1702+1726) 2mm_2_3 | 25 mer |
| 175 | CAGAACTAACCCTGTAAAGTTAAAA | SHANK3 H22A(+1702+1726) 2mm_2_8 | 25 mer |
| 176 | CGAAACTGACCCTATAAAGTTAAAA | SHANK3 H22A(+1702+1726) 2mm_3_14 | 25 mer |
| 177 | CGAAACTGACCCTGTAAAATTAAAA | SHANK3 H22A(+1702+1726) 2mm_3_19 | 25 mer |
| 178 | CGAAACTAACCCTGTAAAGTTAAAA | SHANK3 H22A(+1702+1726) 2mm_3_8 | 25 mer |
| 179 | CGGAACTAACCCTGTAAAATTAAAA | SHANK3 H22A(+1702+1726) 2mm_8_19 | 25 mer |
| 180 | CGGAACTGACCCTGTAAAATTAAAA | SHANK3 H22A(+1702+1726) mm1 | 25 mer |
| 181 | CGGAACTGACCCTATAAAATTAAAA | SHANK3 H22A(+1702+1726) mm2 | 25 mer |
| 182 | GCGGAACTGACCCTGTAAAGTTAAA | SHANK3 H22A(+1703+1727) | 25 mer |
| 183 | TCGGAACTGACCCTGTAAAGTTAAA | SHANK3 H22A(+1703+1727) 1mm_1 | 25 mer |
| 184 | GCGGAACTGATCCTGTAAAGTTAAA | SHANK3 H22A(+1703+1727) 1mm_11 | 25 mer |
| 185 | GCGGAACTGACTCTGTAAAGTTAAA | SHANK3 H22A(+1703+1727) 1mm_12 | 25 mer |
| 186 | GCGGAACTGACCTTGTAAAGTTAAA | SHANK3 H22A(+1703+1727) 1mm_13 | 25 mer |
| 187 | GCGGAACTGACCCTTTAAAGTTAAA | SHANK3 H22A(+1703+1727) 1mm_15 | 25 mer |
| 188 | GTGGAACTGACCCTGTAAAGTTAAA | SHANK3 H22A(+1703+1727) 1mm_2 | 25 mer |
| 189 | GCGGAACTGACCCTGTAAATTTAAA | SHANK3 H22A(+1703+1727) 1mm_20 | 25 mer |
| 190 | GCTGAACTGACCCTGTAAAGTTAAA | SHANK3 H22A(+1703+1727) 1mm_3 | 25 mer |
| 191 | GCGTAACTGACCCTGTAAAGTTAAA | SHANK3 H22A(+1703+1727) 1mm_4 | 25 mer |
| 192 | GCGGAATTGACCCTGTAAAGTTAAA | SHANK3 H22A(+1703+1727) 1mm_7 | 25 mer |
| 193 | GCGGAACTTACCCTGTAAAGTTAAA | SHANK3 H22A(+1703+1727) 1mm_9 | 25 mer |
| 194 | ACGGAACTGACCCTATAAAGTTAAA | SHANK3 H22A(+1703+1727) 2mm_1_15 | 25 mer |
| 195 | ACGGAACTGACCCTGTAAAATTAAA | SHANK3 H22A(+1703+1727) 2mm_1_20 | 25 mer |
| 196 | ACAGAACTGACCCTGTAAAGTTAAA | SHANK3 H22A(+1703+1727) 2mm_1_3 | 25 mer |
| 197 | ACGAAACTGACCCTGTAAAGTTAAA | SHANK3 H22A(+1703+1727) 2mm_1_4 | 25 mer |
| 198 | ACGGAACTAACCCTGTAAAGTTAAA | SHANK3 H22A(+1703+1727) 2mm_1_9 | 25 mer |
| 199 | GCAGAACTGACCCTATAAAGTTAAA | SHANK3 H22A(+1703+1727) 2mm_3_15 | 25 mer |
| 200 | GCAGAACTGACCCTGTAAAATTAAA | SHANK3 H22A(+1703+1727) 2mm_3_20 | 25 mer |
| 201 | GCAAAACTGACCCTGTAAAGTTAAA | SHANK3 H22A(+1703+1727) 2mm_3_4 | 25 mer |
| 202 | GCAGAACTAACCCTGTAAAGTTAAA | SHANK3 H22A(+1703+1727) 2mm_3_9 | 25 mer |
| 203 | GCGAAACTGACCCTATAAAGTTAAA | SHANK3 H22A(+1703+1727) 2mm_4_15 | 25 mer |
| 204 | GCGAAACTGACCCTGTAAAATTAAA | SHANK3 H22A(+1703+1727) 2mm_4_20 | 25 mer |
| 205 | GCGAAACTAACCCTGTAAAGTTAAA | SHANK3 H22A(+1703+1727) 2mm_4_9 | 25 mer |
| 206 | GCGGAACTAACCCTGTAAAATTAAA | SHANK3 H22A(+1703+1727) 2mm_9_20 | 25 mer |
| 207 | GCGGAACTGACCCTGTAAAATTAAA | SHANK3 H22A(+1703+1727) mm1 | 25 mer |
| 208 | GCGGAACTGACCCTATAAAATTAAA | SHANK3 H22A(+1703+1727) mm2 | 25 mer |
| 209 | AGCGGAACTGACCCTGTAAAGTTAA | SHANK3 H22A(+1704+1728) | 25 mer |
| 210 | AGCGGAACTTACCCTGTAAAGTTAA | SHANK3 H22A(+1704+1728) 1mm_10 | 25 mer |
| 211 | AGCGGAACTGATCCTGTAAAGTTAA | SHANK3 H22A(+1704+1728) 1mm_12 | 25 mer |
| 212 | AGCGGAACTGACTCTGTAAAGTTAA | SHANK3 H22A(+1704+1728) 1mm_13 | 25 mer |
| 213 | AGCGGAACTGACCTTGTAAAGTTAA | SHANK3 H22A(+1704+1728) 1mm_14 | 25 mer |
| 214 | AGCGGAACTGACCCTTTAAAGTTAA | SHANK3 H22A(+1704+1728) 1mm_16 | 25 mer |
| 215 | ATCGGAACTGACCCTGTAAAGTTAA | SHANK3 H22A(+1704+1728) 1mm_2 | 25 mer |
| 216 | AGCGGAACTGACCCTGTAAATTTAA | SHANK3 H22A(+1704+1728) 1mm_21 | 25 mer |
| 217 | AGTGGAACTGACCCTGTAAAGTTAA | SHANK3 H22A(+1704+1728) 1mm_3 | 25 mer |
| 218 | AGCTGAACTGACCCTGTAAAGTTAA | SHANK3 H22A(+1704+1728) 1mm_4 | 25 mer |
| 219 | AGCGTAACTGACCCTGTAAAGTTAA | SHANK3 H22A(+1704+1728) 1mm_5 | 25 mer |
| 220 | AGCGGAATTGACCCTGTAAAGTTAA | SHANK3 H22A(+1704+1728) 1mm_8 | 25 mer |
| 221 | AGCGGAACTAACCCTGTAAAATTAA | SHANK3 H22A(+1704+1728) 2mm_10_21 | 25 mer |
| 222 | AACGGAACTAACCCTGTAAAGTTAA | SHANK3 H22A(+1704+1728) 2mm_2_10 | 25 mer |
| 223 | AACGGAACTGACCCTATAAAGTTAA | SHANK3 H22A(+1704+1728) 2mm_2_16 | 25 mer |
| 224 | AACGGAACTGACCCTGTAAAATTAA | SHANK3 H22A(+1704+1728) 2mm_2_21 | 25 mer |
| 225 | AACAGAACTGACCCTGTAAAGTTAA | SHANK3 H22A(+1704+1728) 2mm_2_4 | 25 mer |
| 226 | AACGAAACTGACCCTGTAAAGTTAA | SHANK3 H22A(+1704+1728) 2mm_2_5 | 25 mer |
| 227 | AGCAGAACTAACCCTGTAAAGTTAA | SHANK3 H22A(+1704+1728) 2mm_4_10 | 25 mer |
| 228 | AGCAGAACTGACCCTATAAAGTTAA | SHANK3 H22A(+1704+1728) 2mm_4_16 | 25 mer |
| 229 | AGCAGAACTGACCCTGTAAAATTAA | SHANK3 H22A(+1704+1728) 2mm_4_21 | 25 mer |
| 230 | AGCAAAACTGACCCTGTAAAGTTAA | SHANK3 H22A(+1704+1728) 2mm_4_5 | 25 mer |
| 231 | AGCGAAACTAACCCTGTAAAGTTAA | SHANK3 H22A(+1704+1728) 2mm_5_10 | 25 mer |
| 232 | AGCGAAACTGACCCTATAAAGTTAA | SHANK3 H22A(+1704+1728) 2mm_5_16 | 25 mer |
| 233 | AGCGAAACTGACCCTGTAAAATTAA | SHANK3 H22A(+1704+1728) 2mm_5_21 | 25 mer |
| 234 | AGCGGAACTGACCCTGTAAAATTAA | SHANK3 H22A(+1704+1728) mm1 | 25 mer |
| 235 | AGCGGAACTGACCCTATAAAATTAA | SHANK3 H22A(+1704+1728) mm2 | 25 mer |
| 236 | AAGCGGAACTTACCCTGTAAAGTTA | SHANK3 H22A(+1705+1729) 1mm_11 | 25 mer |
| 237 | AAGCGGAACTGATCCTGTAAAGTTA | SHANK3 H22A(+1705+1729) 1mm_13 | 25 mer |
| 238 | AAGCGGAACTGACTCTGTAAAGTTA | SHANK3 H22A(+1705+1729) 1mm_14 | 25 mer |
| 239 | AAGCGGAACTGACCTTGTAAAGTTA | SHANK3 H22A(+1705+1729) 1mm_15 | 25 mer |
| 240 | AAGCGGAACTGACCCTTTAAAGTTA | SHANK3 H22A(+1705+1729) 1mm_17 | 25 mer |
| 241 | AAGCGGAACTGACCCTGTAAATTTA | SHANK3 H22A(+1705+1729) 1mm_22 | 25 mer |
| 242 | AATCGGAACTGACCCTGTAAAGTTA | SHANK3 H22A(+1705+1729) 1mm_3 | 25 mer |
| 243 | AAGTGGAACTGACCCTGTAAAGTTA | SHANK3 H22A(+1705+1729) 1mm_4 | 25 mer |
| 244 | AAGCTGAACTGACCCTGTAAAGTTA | SHANK3 H22A(+1705+1729) 1mm_5 | 25 mer |
| 245 | AAGCGTAACTGACCCTGTAAAGTTA | SHANK3 H22A(+1705+1729) 1mm_6 | 25 mer |
| 246 | AAGCGGAATTGACCCTGTAAAGTTA | SHANK3 H22A(+1705+1729) 1mm_9 | 25 mer |
| 247 | AAGCGGAACTAACCCTGTAAAATTA | SHANK3 H22A(+1705+1729) 2mm_11_22 | 25 mer |
| 248 | AAACGGAACTAACCCTGTAAAGTTA | SHANK3 H22A(+1705+1729) 2mm_3_11 | 25 mer |
| 249 | AAACGGAACTGACCCTATAAAGTTA | SHANK3 H22A(+1705+1729) 2mm_3_17 | 25 mer |
| 250 | AAACGGAACTGACCCTGTAAAATTA | SHANK3 H22A(+1705+1729) 2mm_3_22 | 25 mer |
| 251 | AAACAGAACTGACCCTGTAAAGTTA | SHANK3 H22A(+1705+1729) 2mm_3_5 | 25 mer |
| 252 | AAACGAAACTGACCCTGTAAAGTTA | SHANK3 H22A(+1705+1729) 2mm_3_6 | 25 mer |
| 253 | AAGCAGAACTAACCCTGTAAAGTTA | SHANK3 H22A(+1705+1729) 2mm_5_11 | 25 mer |
| 254 | AAGCAGAACTGACCCTATAAAGTTA | SHANK3 H22A(+1705+1729) 2mm_5_17 | 25 mer |
| 255 | AAGCAGAACTGACCCTGTAAAATTA | SHANK3 H22A(+1705+1729) 2mm_5_22 | 25 mer |
| 256 | AAGCAAAACTGACCCTGTAAAGTTA | SHANK3 H22A(+1705+1729) 2mm_5_6 | 25 mer |
| 257 | AAGCGAAACTAACCCTGTAAAGTTA | SHANK3 H22A(+1705+1729) 2mm_6_11 | 25 mer |
| 258 | AAGCGAAACTGACCCTATAAAGTTA | SHANK3 H22A(+1705+1729) 2mm_6_17 | 25 mer |
| 259 | AAGCGAAACTGACCCTGTAAAATTA | SHANK3 H22A(+1705+1729) 2mm_6_22 | 25 mer |
| 260 | AAGCGGAACTGACCCTGTAAAATTA | SHANK3 H22A(+1705+1729) mm1 | 25 mer |
| 261 | AAGCGGAACTGACCCTATAAAATTA | SHANK3 H22A(+1705+1729) mm2 | 25 mer |
| 262 | GAAGCGGAACTGACCCTGTAAAGTT | SHANK3 H22A(+1706+1730) | 25 mer |
| 263 | TAAGCGGAACTGACCCTGTAAAGTT | SHANK3 H22A(+1706+1730) 1mm_1 | 25 mer |
| 264 | GAAGCGGAATTGACCCTGTAAAGTT | SHANK3 H22A(+1706+1730) 1mm_10 | 25 mer |
| 265 | GAAGCGGAACTTACCCTGTAAAGTT | SHANK3 H22A(+1706+1730) 1mm_12 | 25 mer |
| 266 | GAAGCGGAACTGATCCTGTAAAGTT | SHANK3 H22A(+1706+1730) 1mm_14 | 25 mer |
| 267 | GAAGCGGAACTGACTCTGTAAAGTT | SHANK3 H22A(+1706+1730) 1mm_15 | 25 mer |
| 268 | GAAGCGGAACTGACCTTGTAAAGTT | SHANK3 H22A(+1706+1730) 1mm_16 | 25 mer |
| 269 | GAAGCGGAACTGACCCTTTAAAGTT | SHANK3 H22A(+1706+1730) 1mm_18 | 25 mer |
| 270 | GAAGCGGAACTGACCCTGTAAATTT | SHANK3 H22A(+1706+1730) 1mm_23 | 25 mer |
| 271 | GAATCGGAACTGACCCTGTAAAGTT | SHANK3 H22A(+1706+1730) 1mm_4 | 25 mer |
| 272 | GAAGTGGAACTGACCCTGTAAAGTT | SHANK3 H22A(+1706+1730) 1mm_5 | 25 mer |
| 273 | GAAGCTGAACTGACCCTGTAAAGTT | SHANK3 H22A(+1706+1730) 1mm_6 | 25 mer |
| 274 | GAAGCGTAACTGACCCTGTAAAGTT | SHANK3 H22A(+1706+1730) 1mm_7 | 25 mer |
| 275 | AAAGCGGAACTAACCCTGTAAAGTT | SHANK3 H22A(+1706+1730) 2mm_1_12 | 25 mer |
| 276 | AAAGCGGAACTGACCCTATAAAGTT | SHANK3 H22A(+1706+1730) 2mm_1_18 | 25 mer |
| 277 | AAAGCGGAACTGACCCTGTAAAATT | SHANK3 H22A(+1706+1730) 2mm_1_23 | 25 mer |
| 278 | AAAACGGAACTGACCCTGTAAAGTT | SHANK3 H22A(+1706+1730) 2mm_1_4 | 25 mer |
| 279 | AAAGCAGAACTGACCCTGTAAAGTT | SHANK3 H22A(+1706+1730) 2mm_1_6 | 25 mer |
| 280 | AAAGCGAAACTGACCCTGTAAAGTT | SHANK3 H22A(+1706+1730) 2mm_1_7 | 25 mer |
| 281 | GAAGCGGAACTAACCCTGTAAAATT | SHANK3 H22A(+1706+1730) 2mm_12_23 | 25 mer |
| 282 | GAAACGGAACTAACCCTGTAAAGTT | SHANK3 H22A(+1706+1730) 2mm_4_12 | 25 mer |
| 283 | GAAACGGAACTGACCCTATAAAGTT | SHANK3 H22A(+1706+1730) 2mm_4_18 | 25 mer |
| 284 | GAAACGGAACTGACCCTGTAAAATT | SHANK3 H22A(+1706+1730) 2mm_4_23 | 25 mer |
| 285 | GAAACAGAACTGACCCTGTAAAGTT | SHANK3 H22A(+1706+1730) 2mm_4_6 | 25 mer |
| 286 | GAAACGAAACTGACCCTGTAAAGTT | SHANK3 H22A(+1706+1730) 2mm_4_7 | 25 mer |
| 287 | GAAGCAGAACTAACCCTGTAAAGTT | SHANK3 H22A(+1706+1730) 2mm_6_12 | 25 mer |
| 288 | GAAGCAGAACTGACCCTATAAAGTT | SHANK3 H22A(+1706+1730) 2mm_6_18 | 25 mer |
| 289 | GAAGCAGAACTGACCCTGTAAAATT | SHANK3 H22A(+1706+1730) 2mm_6_23 | 25 mer |
| 290 | GAAGCAAAACTGACCCTGTAAAGTT | SHANK3 H22A(+1706+1730) 2mm_6_7 | 25 mer |
| 291 | GAAGCGAAACTAACCCTGTAAAGTT | SHANK3 H22A(+1706+1730) 2mm_7_12 | 25 mer |
| 292 | GAAGCGAAACTGACCCTATAAAGTT | SHANK3 H22A(+1706+1730) 2mm_7_18 | 25 mer |
| 293 | GAAGCGAAACTGACCCTGTAAAATT | SHANK3 H22A(+1706+1730) 2mm_7_23 | 25 mer |
| 294 | GAAGCGGAACTGACCCTGTAAAATT | SHANK3 H22A(+1706+1730) mm1 | 25 mer |
| 295 | GAAGCGGAACTGACCCTATAAAATT | SHANK3 H22A(+1706+1730) mm2 | 25 mer |
| 296 | TGAAGCGGAATTGACCCTGTAAAGT | SHANK3 H22A(+1707+1731) 1mm_11 | 25 mer |
| 297 | TGAAGCGGAACTTACCCTGTAAAGT | SHANK3 H22A(+1707+1731) 1mm_13 | 25 mer |
| 298 | TGAAGCGGAACTGATCCTGTAAAGT | SHANK3 H22A(+1707+1731) 1mm_15 | 25 mer |
| 299 | TGAAGCGGAACTGACTCTGTAAAGT | SHANK3 H22A(+1707+1731) 1mm_16 | 25 mer |
| 300 | TGAAGCGGAACTGACCTTGTAAAGT | SHANK3 H22A(+1707+1731) 1mm_17 | 25 mer |
| 301 | TGAAGCGGAACTGACCCTTTAAAGT | SHANK3 H22A(+1707+1731) 1mm_19 | 25 mer |
| 302 | TTAAGCGGAACTGACCCTGTAAAGT | SHANK3 H22A(+1707+1731) 1mm_2 | 25 mer |
| 303 | TGAAGCGGAACTGACCCTGTAAATT | SHANK3 H22A(+1707+1731) 1mm_24 | 25 mer |
| 304 | TGAATCGGAACTGACCCTGTAAAGT | SHANK3 H22A(+1707+1731) 1mm_5 | 25 mer |
| 305 | TGAAGTGGAACTGACCCTGTAAAGT | SHANK3 H22A(+1707+1731) 1mm_6 | 25 mer |
| 306 | TGAAGCTGAACTGACCCTGTAAAGT | SHANK3 H22A(+1707+1731) 1mm_7 | 25 mer |
| 307 | TGAAGCGTAACTGACCCTGTAAAGT | SHANK3 H22A(+1707+1731) 1mm_8 | 25 mer |
| 308 | TGAAGCGGAACTAACCCTGTAAAAT | SHANK3 H22A(+1707+1731) 2mm_13_24 | 25 mer |
| 309 | TAAAGCGGAACTAACCCTGTAAAGT | SHANK3 H22A(+1707+1731) 2mm_2_13 | 25 mer |
| 310 | TAAAGCGGAACTGACCCTATAAAGT | SHANK3 H22A(+1707+1731) 2mm_2_19 | 25 mer |
| 311 | TAAAGCGGAACTGACCCTGTAAAAT | SHANK3 H22A(+1707+1731) 2mm_2_24 | 25 mer |
| 312 | TAAAACGGAACTGACCCTGTAAAGT | SHANK3 H22A(+1707+1731) 2mm_2_5 | 25 mer |
| 313 | TAAAGCAGAACTGACCCTGTAAAGT | SHANK3 H22A(+1707+1731) 2mm_2_7 | 25 mer |
| 314 | TAAAGCGAAACTGACCCTGTAAAGT | SHANK3 H22A(+1707+1731) 2mm_2_8 | 25 mer |
| 315 | TGAAACGGAACTAACCCTGTAAAGT | SHANK3 H22A(+1707+1731) 2mm_5_13 | 25 mer |
| 316 | TGAAACGGAACTGACCCTATAAAGT | SHANK3 H22A(+1707+1731) 2mm_5_19 | 25 mer |
| 317 | TGAAACGGAACTGACCCTGTAAAAT | SHANK3 H22A(+1707+1731) 2mm_5_24 | 25 mer |
| 318 | TGAAACAGAACTGACCCTGTAAAGT | SHANK3 H22A(+1707+1731) 2mm_5_7 | 25 mer |
| 319 | TGAAACGAAACTGACCCTGTAAAGT | SHANK3 H22A(+1707+1731) 2mm_5_8 | 25 mer |
| 320 | TGAAGCAGAACTAACCCTGTAAAGT | SHANK3 H22A(+1707+1731) 2mm_7_13 | 25 mer |
| 321 | TGAAGCAGAACTGACCCTATAAAGT | SHANK3 H22A(+1707+1731) 2mm_7_19 | 25 mer |
| 322 | TGAAGCAGAACTGACCCTGTAAAAT | SHANK3 H22A(+1707+1731) 2mm_7_24 | 25 mer |
| 323 | TGAAGCAAAACTGACCCTGTAAAGT | SHANK3 H22A(+1707+1731) 2mm_7_8 | 25 mer |
| 324 | TGAAGCGAAACTAACCCTGTAAAGT | SHANK3 H22A(+1707+1731) 2mm_8_13 | 25 mer |
| 325 | TGAAGCGAAACTGACCCTATAAAGT | SHANK3 H22A(+1707+1731) 2mm_8_19 | 25 mer |
| 326 | TGAAGCGAAACTGACCCTGTAAAAT | SHANK3 H22A(+1707+1731) 2mm_8_24 | 25 mer |
| 327 | TGAAGCGGAACTGACCCTGTAAAAT | SHANK3 H22A(+1707+1731) mm1 | 25 mer |
| 328 | TGAAGCGGAACTGACCCTATAAAAT | SHANK3 H22A(+1707+1731) mm2 | 25 mer |
| 329 | TTGAAGCGGAACTGACCCTGTAAAG | SHANK3 H22A(+1708+1732) 1mm_1 | 25 mer |
| 330 | GTGAAGCGGAATTGACCCTGTAAAG | SHANK3 H22A(+1708+1732) 1mm_12 | 25 mer |
| 331 | GTGAAGCGGAACTTACCCTGTAAAG | SHANK3 H22A(+1708+1732) 1mm_14 | 25 mer |
| 332 | GTGAAGCGGAACTGATCCTGTAAAG | SHANK3 H22A(+1708+1732) 1mm_16 | 25 mer |
| 333 | GTGAAGCGGAACTGACTCTGTAAAG | SHANK3 H22A(+1708+1732) 1mm_17 | 25 mer |
| 334 | GTGAAGCGGAACTGACCTTGTAAAG | SHANK3 H22A(+1708+1732) 1mm_18 | 25 mer |
| 335 | GTGAAGCGGAACTGACCCTTTAAAG | SHANK3 H22A(+1708+1732) 1mm_20 | 25 mer |
| 336 | GTTAAGCGGAACTGACCCTGTAAAG | SHANK3 H22A(+1708+1732) 1mm_3 | 25 mer |
| 337 | GTGAATCGGAACTGACCCTGTAAAG | SHANK3 H22A(+1708+1732) 1mm_6 | 25 mer |
| 338 | GTGAAGTGGAACTGACCCTGTAAAG | SHANK3 H22A(+1708+1732) 1mm_7 | 25 mer |
| 339 | GTGAAGCTGAACTGACCCTGTAAAG | SHANK3 H22A(+1708+1732) 1mm_8 | 25 mer |
| 340 | GTGAAGCGTAACTGACCCTGTAAAG | SHANK3 H22A(+1708+1732) 1mm_9 | 25 mer |
| 341 | ATGAAGCGGAACTAACCCTGTAAAG | SHANK3 H22A(+1708+1732) 2mm_1_14 | 25 mer |
| 342 | ATGAAGCGGAACTGACCCTATAAAG | SHANK3 H22A(+1708+1732) 2mm_1_20 | 25 mer |
| 343 | ATGAAGCGGAACTGACCCTGTAAAA | SHANK3 H22A(+1708+1732) 2mm_1_25 | 25 mer |
| 344 | ATAAAGCGGAACTGACCCTGTAAAG | SHANK3 H22A(+1708+1732) 2mm_1_3 | 25 mer |
| 345 | ATGAAACGGAACTGACCCTGTAAAG | SHANK3 H22A(+1708+1732) 2mm_1_6 | 25 mer |
| 346 | ATGAAGCAGAACTGACCCTGTAAAG | SHANK3 H22A(+1708+1732) 2mm_1_8 | 25 mer |
| 347 | ATGAAGCGAAACTGACCCTGTAAAG | SHANK3 H22A(+1708+1732) 2mm_1_9 | 25 mer |
| 348 | GTGAAGCGGAACTAACCCTGTAAAA | SHANK3 H22A(+1708+1732) 2mm_14_25 | 25 mer |
| 349 | GTAAAGCGGAACTAACCCTGTAAAG | SHANK3 H22A(+1708+1732) 2mm_3_14 | 25 mer |
| 350 | GTAAAGCGGAACTGACCCTATAAAG | SHANK3 H22A(+1708+1732) 2mm_3_20 | 25 mer |
| 351 | GTAAAGCGGAACTGACCCTGTAAAA | SHANK3 H22A(+1708+1732) 2mm_3_25 | 25 mer |
| 352 | GTAAAACGGAACTGACCCTGTAAAG | SHANK3 H22A(+1708+1732) 2mm_3_6 | 25 mer |
| 353 | GTAAAGCAGAACTGACCCTGTAAAG | SHANK3 H22A(+1708+1732) 2mm_3_8 | 25 mer |
| 354 | GTAAAGCGAAACTGACCCTGTAAAG | SHANK3 H22A(+1708+1732) 2mm_3_9 | 25 mer |
| 355 | GTGAAACGGAACTAACCCTGTAAAG | SHANK3 H22A(+1708+1732) 2mm_6_14 | 25 mer |
| 356 | GTGAAACGGAACTGACCCTATAAAG | SHANK3 H22A(+1708+1732) 2mm_6_20 | 25 mer |
| 357 | GTGAAACGGAACTGACCCTGTAAAA | SHANK3 H22A(+1708+1732) 2mm_6_25 | 25 mer |
| 358 | GTGAAACAGAACTGACCCTGTAAAG | SHANK3 H22A(+1708+1732) 2mm_6_8 | 25 mer |
| 359 | GTGAAACGAAACTGACCCTGTAAAG | SHANK3 H22A(+1708+1732) 2mm_6_9 | 25 mer |
| 360 | GTGAAGCAGAACTAACCCTGTAAAG | SHANK3 H22A(+1708+1732) 2mm_8_14 | 25 mer |
| 361 | GTGAAGCAGAACTGACCCTATAAAG | SHANK3 H22A(+1708+1732) 2mm_8_20 | 25 mer |
| 362 | GTGAAGCAGAACTGACCCTGTAAAA | SHANK3 H22A(+1708+1732) 2mm_8_25 | 25 mer |
| 363 | GTGAAGCAAAACTGACCCTGTAAAG | SHANK3 H22A(+1708+1732) 2mm_8_9 | 25 mer |
| 364 | GTGAAGCGAAACTAACCCTGTAAAG | SHANK3 H22A(+1708+1732) 2mm_9_14 | 25 mer |
| 365 | GTGAAGCGAAACTGACCCTATAAAG | SHANK3 H22A(+1708+1732) 2mm_9_20 | 25 mer |
| 366 | GTGAAGCGAAACTGACCCTGTAAAA | SHANK3 H22A(+1708+1732) 2mm_9_25 | 25 mer |
| 367 | GTGAAGCGGAACTGACCCTGTAAAA | SHANK3 H22A(+1708+1732) mm1 | 25 mer |
| 368 | GTGAAGCGGAACTGACCCTATAAAA | SHANK3 H22A(+1708+1732) mm2 | 25 mer |
| 369 | GGTGAAGCGGAACTGACCCTGTAAA | SHANK3 H22A(+1709+1733) | 25 mer |
| 370 | TGTGAAGCGGAACTGACCCTGTAAA | SHANK3 H22A(+1709+1733) 1mm_1 | 25 mer |
| 371 | GGTGAAGCGTAACTGACCCTGTAAA | SHANK3 H22A(+1709+1733) 1mm_10 | 25 mer |
| 372 | GGTGAAGCGGAATTGACCCTGTAAA | SHANK3 H22A(+1709+1733) 1mm_13 | 25 mer |
| 373 | GGTGAAGCGGAACTTACCCTGTAAA | SHANK3 H22A(+1709+1733) 1mm_15 | 25 mer |
| 374 | GGTGAAGCGGAACTGATCCTGTAAA | SHANK3 H22A(+1709+1733) 1mm_17 | 25 mer |
| 375 | GGTGAAGCGGAACTGACTCTGTAAA | SHANK3 H22A(+1709+1733) 1mm_18 | 25 mer |
| 376 | GGTGAAGCGGAACTGACCTTGTAAA | SHANK3 H22A(+1709+1733) 1mm_19 | 25 mer |
| 377 | GTTGAAGCGGAACTGACCCTGTAAA | SHANK3 H22A(+1709+1733) 1mm_2 | 25 mer |
| 378 | GGTGAAGCGGAACTGACCCTTTAAA | SHANK3 H22A(+1709+1733) 1mm_21 | 25 mer |
| 379 | GGTTAAGCGGAACTGACCCTGTAAA | SHANK3 H22A(+1709+1733) 1mm_4 | 25 mer |
| 380 | GGTGAATCGGAACTGACCCTGTAAA | SHANK3 H22A(+1709+1733) 1mm_7 | 25 mer |
| 381 | GGTGAAGTGGAACTGACCCTGTAAA | SHANK3 H22A(+1709+1733) 1mm_8 | 25 mer |
| 382 | GGTGAAGCTGAACTGACCCTGTAAA | SHANK3 H22A(+1709+1733) 1mm_9 | 25 mer |
| 383 | AGTGAAGCGAAACTGACCCTGTAAA | SHANK3 H22A(+1709+1733) 2mm_1_10 | 25 mer |
| 384 | AGTGAAGCGGAACTAACCCTGTAAA | SHANK3 H22A(+1709+1733) 2mm_1_15 | 25 mer |
| 385 | AATGAAGCGGAACTGACCCTGTAAA | SHANK3 H22A(+1709+1733) 2mm_1_2 | 25 mer |
| 386 | AGTGAAGCGGAACTGACCCTATAAA | SHANK3 H22A(+1709+1733) 2mm_1_21 | 25 mer |
| 387 | AGTAAAGCGGAACTGACCCTGTAAA | SHANK3 H22A(+1709+1733) 2mm_1_4 | 25 mer |
| 388 | AGTGAAACGGAACTGACCCTGTAAA | SHANK3 H22A(+1709+1733) 2mm_1_7 | 25 mer |
| 389 | AGTGAAGCAGAACTGACCCTGTAAA | SHANK3 H22A(+1709+1733) 2mm_1_9 | 25 mer |
| 390 | GGTGAAGCGAAACTAACCCTGTAAA | SHANK3 H22A(+1709+1733) 2mm_10_15 | 25 mer |
| 391 | GGTGAAGCGAAACTGACCCTATAAA | SHANK3 H22A(+1709+1733) 2mm_10_21 | 25 mer |
| 392 | GATGAAGCGAAACTGACCCTGTAAA | SHANK3 H22A(+1709+1733) 2mm_2_10 | 25 mer |
| 393 | GATGAAGCGGAACTAACCCTGTAAA | SHANK3 H22A(+1709+1733) 2mm_2_15 | 25 mer |
| 394 | GATGAAGCGGAACTGACCCTATAAA | SHANK3 H22A(+1709+1733) 2mm_2_21 | 25 mer |
| 395 | GATAAAGCGGAACTGACCCTGTAAA | SHANK3 H22A(+1709+1733) 2mm_2_4 | 25 mer |
| 396 | GATGAAACGGAACTGACCCTGTAAA | SHANK3 H22A(+1709+1733) 2mm_2_7 | 25 mer |
| 397 | GATGAAGCAGAACTGACCCTGTAAA | SHANK3 H22A(+1709+1733) 2mm_2_9 | 25 mer |
| 398 | GGTAAAGCGAAACTGACCCTGTAAA | SHANK3 H22A(+1709+1733) 2mm_4_10 | 25 mer |
| 399 | GGTAAAGCGGAACTAACCCTGTAAA | SHANK3 H22A(+1709+1733) 2mm_4_15 | 25 mer |
| 400 | GGTAAAGCGGAACTGACCCTATAAA | SHANK3 H22A(+1709+1733) 2mm_4_21 | 25 mer |
| 401 | GGTAAAACGGAACTGACCCTGTAAA | SHANK3 H22A(+1709+1733) 2mm_4_7 | 25 mer |
| 402 | GGTAAAGCAGAACTGACCCTGTAAA | SHANK3 H22A(+1709+1733) 2mm_4_9 | 25 mer |
| 403 | GGTGAAACGAAACTGACCCTGTAAA | SHANK3 H22A(+1709+1733) 2mm_7_10 | 25 mer |
| 404 | GGTGAAACGGAACTAACCCTGTAAA | SHANK3 H22A(+1709+1733) 2mm_7_15 | 25 mer |
| 405 | GGTGAAACGGAACTGACCCTATAAA | SHANK3 H22A(+1709+1733) 2mm_7_21 | 25 mer |
| 406 | GGTGAAACAGAACTGACCCTGTAAA | SHANK3 H22A(+1709+1733) 2mm_7_9 | 25 mer |
| 407 | GGTGAAGCAAAACTGACCCTGTAAA | SHANK3 H22A(+1709+1733) 2mm_9_10 | 25 mer |
| 408 | GGTGAAGCAGAACTAACCCTGTAAA | SHANK3 H22A(+1709+1733) 2mm_9_15 | 25 mer |
| 409 | GGTGAAGCAGAACTGACCCTATAAA | SHANK3 H22A(+1709+1733) 2mm_9_21 | 25 mer |
| 410 | GGTGAAGCGGAACTGACCCTATAAA | SHANK3 H22A(+1709+1733) mm1 | 25 mer |
| 411 | AGGTGAAGCGGAACTGACCCTGTAA | SHANK3 H22A(+1710+1734) | 25 mer |
| 412 | AGGTGAAGCTGAACTGACCCTGTAA | SHANK3 H22A(+1710+1734) 1mm_10 | 25 mer |
| 413 | AGGTGAAGCGTAACTGACCCTGTAA | SHANK3 H22A(+1710+1734) 1mm_11 | 25 mer |
| 414 | AGGTGAAGCGGAATTGACCCTGTAA | SHANK3 H22A(+1710+1734) 1mm_14 | 25 mer |
| 415 | AGGTGAAGCGGAACTTACCCTGTAA | SHANK3 H22A(+1710+1734) 1mm_16 | 25 mer |
| 416 | AGGTGAAGCGGAACTGATCCTGTAA | SHANK3 H22A(+1710+1734) 1mm_18 | 25 mer |
| 417 | AGGTGAAGCGGAACTGACTCTGTAA | SHANK3 H22A(+1710+1734) 1mm_19 | 25 mer |
| 418 | ATGTGAAGCGGAACTGACCCTGTAA | SHANK3 H22A(+1710+1734) 1mm_2 | 25 mer |
| 419 | AGGTGAAGCGGAACTGACCTTGTAA | SHANK3 H22A(+1710+1734) 1mm_20 | 25 mer |
| 420 | AGGTGAAGCGGAACTGACCCTTTAA | SHANK3 H22A(+1710+1734) 1mm_22 | 25 mer |
| 421 | AGTTGAAGCGGAACTGACCCTGTAA | SHANK3 H22A(+1710+1734) 1mm_3 | 25 mer |
| 422 | AGGTTAAGCGGAACTGACCCTGTAA | SHANK3 H22A(+1710+1734) 1mm_5 | 25 mer |
| 423 | AGGTGAATCGGAACTGACCCTGTAA | SHANK3 H22A(+1710+1734) 1mm_8 | 25 mer |
| 424 | AGGTGAAGTGGAACTGACCCTGTAA | SHANK3 H22A(+1710+1734) 1mm_9 | 25 mer |
| 425 | AGGTGAAGCAAAACTGACCCTGTAA | SHANK3 H22A(+1710+1734) 2mm_10_11 | 25 mer |
| 426 | AGGTGAAGCAGAACTAACCCTGTAA | SHANK3 H22A(+1710+1734) 2mm_10_16 | 25 mer |
| 427 | AGGTGAAGCAGAACTGACCCTATAA | SHANK3 H22A(+1710+1734) 2mm_10_22 | 25 mer |
| 428 | AGGTGAAGCGAAACTAACCCTGTAA | SHANK3 H22A(+1710+1734) 2mm_11_16 | 25 mer |
| 429 | AGGTGAAGCGAAACTGACCCTATAA | SHANK3 H22A(+1710+1734) 2mm_11_22 | 25 mer |
| 430 | AAGTGAAGCAGAACTGACCCTGTAA | SHANK3 H22A(+1710+1734) 2mm_2_10 | 25 mer |
| 431 | AAGTGAAGCGAAACTGACCCTGTAA | SHANK3 H22A(+1710+1734) 2mm_2_11 | 25 mer |
| 432 | AAGTGAAGCGGAACTAACCCTGTAA | SHANK3 H22A(+1710+1734) 2mm_2_16 | 25 mer |
| 433 | AAGTGAAGCGGAACTGACCCTATAA | SHANK3 H22A(+1710+1734) 2mm_2_22 | 25 mer |
| 434 | AAATGAAGCGGAACTGACCCTGTAA | SHANK3 H22A(+1710+1734) 2mm_2_3 | 25 mer |
| 435 | AAGTAAAGCGGAACTGACCCTGTAA | SHANK3 H22A(+1710+1734) 2mm_2_5 | 25 mer |
| 436 | AAGTGAAACGGAACTGACCCTGTAA | SHANK3 H22A(+1710+1734) 2mm_2_8 | 25 mer |
| 437 | AGATGAAGCAGAACTGACCCTGTAA | SHANK3 H22A(+1710+1734) 2mm_3_10 | 25 mer |
| 438 | AGATGAAGCGAAACTGACCCTGTAA | SHANK3 H22A(+1710+1734) 2mm_3_11 | 25 mer |
| 439 | AGATGAAGCGGAACTAACCCTGTAA | SHANK3 H22A(+1710+1734) 2mm_3_16 | 25 mer |
| 440 | AGATGAAGCGGAACTGACCCTATAA | SHANK3 H22A(+1710+1734) 2mm_3_22 | 25 mer |
| 441 | AGATAAAGCGGAACTGACCCTGTAA | SHANK3 H22A(+1710+1734) 2mm_3_5 | 25 mer |
| 442 | AGATGAAACGGAACTGACCCTGTAA | SHANK3 H22A(+1710+1734) 2mm_3_8 | 25 mer |
| 443 | AGGTAAAGCAGAACTGACCCTGTAA | SHANK3 H22A(+1710+1734) 2mm_5_10 | 25 mer |
| 444 | AGGTAAAGCGAAACTGACCCTGTAA | SHANK3 H22A(+1710+1734) 2mm_5_11 | 25 mer |
| 445 | AGGTAAAGCGGAACTAACCCTGTAA | SHANK3 H22A(+1710+1734) 2mm_5_16 | 25 mer |
| 446 | AGGTAAAGCGGAACTGACCCTATAA | SHANK3 H22A(+1710+1734) 2mm_5_22 | 25 mer |
| 447 | AGGTAAAACGGAACTGACCCTGTAA | SHANK3 H22A(+1710+1734) 2mm_5_8 | 25 mer |
| 448 | AGGTGAAACAGAACTGACCCTGTAA | SHANK3 H22A(+1710+1734) 2mm_8_10 | 25 mer |
| 449 | AGGTGAAACGAAACTGACCCTGTAA | SHANK3 H22A(+1710+1734) 2mm_8_11 | 25 mer |
| 450 | AGGTGAAACGGAACTAACCCTGTAA | SHANK3 H22A(+1710+1734) 2mm_8_16 | 25 mer |
| 451 | AGGTGAAACGGAACTGACCCTATAA | SHANK3 H22A(+1710+1734) 2mm_8_22 | 25 mer |
| 452 | AGGTGAAGCGGAACTGACCCTATAA | SHANK3 H22A(+1710+1734) mm1 | 25 mer |
| 453 | TAGGTGAAGCGGAACTGACCCTGTA | SHANK3 H22A(+1711+1735) 1mm_1 | 25 mer |
| 454 | GAGGTGAAGTGGAACTGACCCTGTA | SHANK3 H22A(+1711+1735) 1mm_10 | 25 mer |
| 455 | GAGGTGAAGCTGAACTGACCCTGTA | SHANK3 H22A(+1711+1735) 1mm_11 | 25 mer |
| 456 | GAGGTGAAGCGTAACTGACCCTGTA | SHANK3 H22A(+1711+1735) 1mm_12 | 25 mer |
| 457 | GAGGTGAAGCGGAATTGACCCTGTA | SHANK3 H22A(+1711+1735) 1mm_15 | 25 mer |
| 458 | GAGGTGAAGCGGAACTTACCCTGTA | SHANK3 H22A(+1711+1735) 1mm_17 | 25 mer |
| 459 | GAGGTGAAGCGGAACTGATCCTGTA | SHANK3 H22A(+1711+1735) 1mm_19 | 25 mer |
| 460 | GAGGTGAAGCGGAACTGACTCTGTA | SHANK3 H22A(+1711+1735) 1mm_20 | 25 mer |
| 461 | GAGGTGAAGCGGAACTGACCTTGTA | SHANK3 H22A(+1711+1735) 1mm_21 | 25 mer |
| 462 | GAGGTGAAGCGGAACTGACCCTTTA | SHANK3 H22A(+1711+1735) 1mm_23 | 25 mer |
| 463 | GATGTGAAGCGGAACTGACCCTGTA | SHANK3 H22A(+1711+1735) 1mm_3 | 25 mer |
| 464 | GAGTTGAAGCGGAACTGACCCTGTA | SHANK3 H22A(+1711+1735) 1mm_4 | 25 mer |
| 465 | GAGGTTAAGCGGAACTGACCCTGTA | SHANK3 H22A(+1711+1735) 1mm_6 | 25 mer |
| 466 | GAGGTGAATCGGAACTGACCCTGTA | SHANK3 H22A(+1711+1735) 1mm_9 | 25 mer |
| 467 | AAGGTGAAGCAGAACTGACCCTGTA | SHANK3 H22A(+1711+1735) 2mm_1_11 | 25 mer |
| 468 | AAGGTGAAGCGAAACTGACCCTGTA | SHANK3 H22A(+1711+1735) 2mm_1_12 | 25 mer |
| 469 | AAGGTGAAGCGGAACTAACCCTGTA | SHANK3 H22A(+1711+1735) 2mm_1_17 | 25 mer |
| 470 | AAGGTGAAGCGGAACTGACCCTATA | SHANK3 H22A(+1711+1735) 2mm_1_23 | 25 mer |
| 471 | AAAGTGAAGCGGAACTGACCCTGTA | SHANK3 H22A(+1711+1735) 2mm_1_3 | 25 mer |
| 472 | AAGATGAAGCGGAACTGACCCTGTA | SHANK3 H22A(+1711+1735) 2mm_1_4 | 25 mer |
| 473 | AAGGTAAAGCGGAACTGACCCTGTA | SHANK3 H22A(+1711+1735) 2mm_1_6 | 25 mer |
| 474 | AAGGTGAAACGGAACTGACCCTGTA | SHANK3 H22A(+1711+1735) 2mm_1_9 | 25 mer |
| 475 | GAGGTGAAGCAAAACTGACCCTGTA | SHANK3 H22A(+1711+1735) 2mm_11_12 | 25 mer |
| 476 | GAGGTGAAGCAGAACTAACCCTGTA | SHANK3 H22A(+1711+1735) 2mm_11_17 | 25 mer |
| 477 | GAGGTGAAGCAGAACTGACCCTATA | SHANK3 H22A(+1711+1735) 2mm_11_23 | 25 mer |
| 478 | GAGGTGAAGCGAAACTAACCCTGTA | SHANK3 H22A(+1711+1735) 2mm_12_17 | 25 mer |
| 479 | GAGGTGAAGCGAAACTGACCCTATA | SHANK3 H22A(+1711+1735) 2mm_12_23 | 25 mer |
| 480 | GAAGTGAAGCAGAACTGACCCTGTA | SHANK3 H22A(+1711+1735) 2mm_3_11 | 25 mer |
| 481 | GAAGTGAAGCGAAACTGACCCTGTA | SHANK3 H22A(+1711+1735) 2mm_3_12 | 25 mer |
| 482 | GAAGTGAAGCGGAACTAACCCTGTA | SHANK3 H22A(+1711+1735) 2mm_3_17 | 25 mer |
| 483 | GAAGTGAAGCGGAACTGACCCTATA | SHANK3 H22A(+1711+1735) 2mm_3_23 | 25 mer |
| 484 | GAAATGAAGCGGAACTGACCCTGTA | SHANK3 H22A(+1711+1735) 2mm_3_4 | 25 mer |
| 485 | GAAGTAAAGCGGAACTGACCCTGTA | SHANK3 H22A(+1711+1735) 2mm_3_6 | 25 mer |
| 486 | GAAGTGAAACGGAACTGACCCTGTA | SHANK3 H22A(+1711+1735) 2mm_3_9 | 25 mer |
| 487 | GAGATGAAGCAGAACTGACCCTGTA | SHANK3 H22A(+1711+1735) 2mm_4_11 | 25 mer |
| 488 | GAGATGAAGCGAAACTGACCCTGTA | SHANK3 H22A(+1711+1735) 2mm_4_12 | 25 mer |
| 489 | GAGATGAAGCGGAACTAACCCTGTA | SHANK3 H22A(+1711+1735) 2mm_4_17 | 25 mer |
| 490 | GAGATGAAGCGGAACTGACCCTATA | SHANK3 H22A(+1711+1735) 2mm_4_23 | 25 mer |
| 491 | GAGATAAAGCGGAACTGACCCTGTA | SHANK3 H22A(+1711+1735) 2mm_4_6 | 25 mer |
| 492 | GAGATGAAACGGAACTGACCCTGTA | SHANK3 H22A(+1711+1735) 2mm_4_9 | 25 mer |
| 493 | GAGGTAAAGCAGAACTGACCCTGTA | SHANK3 H22A(+1711+1735) 2mm_6_11 | 25 mer |
| 494 | GAGGTAAAGCGAAACTGACCCTGTA | SHANK3 H22A(+1711+1735) 2mm_6_12 | 25 mer |
| 495 | GAGGTAAAGCGGAACTAACCCTGTA | SHANK3 H22A(+1711+1735) 2mm_6_17 | 25 mer |
| 496 | GAGGTAAAGCGGAACTGACCCTATA | SHANK3 H22A(+1711+1735) 2mm_6_23 | 25 mer |
| 497 | GAGGTAAAACGGAACTGACCCTGTA | SHANK3 H22A(+1711+1735) 2mm_6_9 | 25 mer |
| 498 | GAGGTGAAACAGAACTGACCCTGTA | SHANK3 H22A(+1711+1735) 2mm_9_11 | 25 mer |
| 499 | GAGGTGAAACGAAACTGACCCTGTA | SHANK3 H22A(+1711+1735) 2mm_9_12 | 25 mer |
| 500 | GAGGTGAAACGGAACTAACCCTGTA | SHANK3 H22A(+1711+1735) 2mm_9_17 | 25 mer |
| 501 | GAGGTGAAACGGAACTGACCCTATA | SHANK3 H22A(+1711+1735) 2mm_9_23 | 25 mer |
| 502 | GAGGTGAAGCGGAACTGACCCTATA | SHANK3 H22A(+1711+1735) mm1 | 25 mer |
| 503 | TGAGGTGAAGCGGAACTGACCCTGT | SHANK3 H22A(+1712+1736) 1mm_1 | 25 mer |
| 504 | GGAGGTGAATCGGAACTGACCCTGT | SHANK3 H22A(+1712+1736) 1mm_10 | 25 mer |
| 505 | GGAGGTGAAGTGGAACTGACCCTGT | SHANK3 H22A(+1712+1736) 1mm_11 | 25 mer |
| 506 | GGAGGTGAAGCTGAACTGACCCTGT | SHANK3 H22A(+1712+1736) 1mm_12 | 25 mer |
| 507 | GGAGGTGAAGCGTAACTGACCCTGT | SHANK3 H22A(+1712+1736) 1mm_13 | 25 mer |
| 508 | GGAGGTGAAGCGGAATTGACCCTGT | SHANK3 H22A(+1712+1736) 1mm_16 | 25 mer |
| 509 | GGAGGTGAAGCGGAACTTACCCTGT | SHANK3 H22A(+1712+1736) 1mm_18 | 25 mer |
| 510 | GTAGGTGAAGCGGAACTGACCCTGT | SHANK3 H22A(+1712+1736) 1mm_2 | 25 mer |
| 511 | GGAGGTGAAGCGGAACTGATCCTGT | SHANK3 H22A(+1712+1736) 1mm_20 | 25 mer |
| 512 | GGAGGTGAAGCGGAACTGACTCTGT | SHANK3 H22A(+1712+1736) 1mm_21 | 25 mer |
| 513 | GGAGGTGAAGCGGAACTGACCTTGT | SHANK3 H22A(+1712+1736) 1mm_22 | 25 mer |
| 514 | GGAGGTGAAGCGGAACTGACCCTTT | SHANK3 H22A(+1712+1736) 1mm_24 | 25 mer |
| 515 | GGATGTGAAGCGGAACTGACCCTGT | SHANK3 H22A(+1712+1736) 1mm_4 | 25 mer |
| 516 | GGAGTTGAAGCGGAACTGACCCTGT | SHANK3 H22A(+1712+1736) 1mm_5 | 25 mer |
| 517 | GGAGGTTAAGCGGAACTGACCCTGT | SHANK3 H22A(+1712+1736) 1mm_7 | 25 mer |
| 518 | AGAGGTGAAACGGAACTGACCCTGT | SHANK3 H22A(+1712+1736) 2mm_1_10 | 25 mer |
| 519 | AGAGGTGAAGCAGAACTGACCCTGT | SHANK3 H22A(+1712+1736) 2mm_1_12 | 25 mer |
| 520 | AGAGGTGAAGCGAAACTGACCCTGT | SHANK3 H22A(+1712+1736) 2mm_1_13 | 25 mer |
| 521 | AGAGGTGAAGCGGAACTAACCCTGT | SHANK3 H22A(+1712+1736) 2mm_1_18 | 25 mer |
| 522 | AAAGGTGAAGCGGAACTGACCCTGT | SHANK3 H22A(+1712+1736) 2mm_1_2 | 25 mer |
| 523 | AGAGGTGAAGCGGAACTGACCCTAT | SHANK3 H22A(+1712+1736) 2mm_1_24 | 25 mer |
| 524 | AGAAGTGAAGCGGAACTGACCCTGT | SHANK3 H22A(+1712+1736) 2mm_1_4 | 25 mer |
| 525 | AGAGATGAAGCGGAACTGACCCTGT | SHANK3 H22A(+1712+1736) 2mm_1_5 | 25 mer |
| 526 | AGAGGTAAAGCGGAACTGACCCTGT | SHANK3 H22A(+1712+1736) 2mm_1_7 | 25 mer |
| 527 | GGAGGTGAAACAGAACTGACCCTGT | SHANK3 H22A(+1712+1736) 2mm_10_12 | 25 mer |
| 528 | GGAGGTGAAACGAAACTGACCCTGT | SHANK3 H22A(+1712+1736) 2mm_10_13 | 25 mer |
| 529 | GGAGGTGAAACGGAACTAACCCTGT | SHANK3 H22A(+1712+1736) 2mm_10_18 | 25 mer |
| 530 | GGAGGTGAAACGGAACTGACCCTAT | SHANK3 H22A(+1712+1736) 2mm_10_24 | 25 mer |
| 531 | GGAGGTGAAGCAAAACTGACCCTGT | SHANK3 H22A(+1712+1736) 2mm_12_13 | 25 mer |
| 532 | GGAGGTGAAGCAGAACTAACCCTGT | SHANK3 H22A(+1712+1736) 2mm_12_18 | 25 mer |
| 533 | GGAGGTGAAGCAGAACTGACCCTAT | SHANK3 H22A(+1712+1736) 2mm_12_24 | 25 mer |
| 534 | GGAGGTGAAGCGAAACTAACCCTGT | SHANK3 H22A(+1712+1736) 2mm_13_18 | 25 mer |
| 535 | GGAGGTGAAGCGAAACTGACCCTAT | SHANK3 H22A(+1712+1736) 2mm_13_24 | 25 mer |
| 536 | GAAGGTGAAACGGAACTGACCCTGT | SHANK3 H22A(+1712+1736) 2mm_2_10 | 25 mer |
| 537 | GAAGGTGAAGCAGAACTGACCCTGT | SHANK3 H22A(+1712+1736) 2mm_2_12 | 25 mer |
| 538 | GAAGGTGAAGCGAAACTGACCCTGT | SHANK3 H22A(+1712+1736) 2mm_2_13 | 25 mer |
| 539 | GAAGGTGAAGCGGAACTAACCCTGT | SHANK3 H22A(+1712+1736) 2mm_2_18 | 25 mer |
| 540 | GAAGGTGAAGCGGAACTGACCCTAT | SHANK3 H22A(+1712+1736) 2mm_2_24 | 25 mer |
| 541 | GAAAGTGAAGCGGAACTGACCCTGT | SHANK3 H22A(+1712+1736) 2mm_2_4 | 25 mer |
| 542 | GAAGATGAAGCGGAACTGACCCTGT | SHANK3 H22A(+1712+1736) 2mm_2_5 | 25 mer |
| 543 | GAAGGTAAAGCGGAACTGACCCTGT | SHANK3 H22A(+1712+1736) 2mm_2_7 | 25 mer |
| 544 | GGAAGTGAAACGGAACTGACCCTGT | SHANK3 H22A(+1712+1736) 2mm_4_10 | 25 mer |
| 545 | GGAAGTGAAGCAGAACTGACCCTGT | SHANK3 H22A(+1712+1736) 2mm_4_12 | 25 mer |
| 546 | GGAAGTGAAGCGAAACTGACCCTGT | SHANK3 H22A(+1712+1736) 2mm_4_13 | 25 mer |
| 547 | GGAAGTGAAGCGGAACTAACCCTGT | SHANK3 H22A(+1712+1736) 2mm_4_18 | 25 mer |
| 548 | GGAAGTGAAGCGGAACTGACCCTAT | SHANK3 H22A(+1712+1736) 2mm_4_24 | 25 mer |
| 549 | GGAAATGAAGCGGAACTGACCCTGT | SHANK3 H22A(+1712+1736) 2mm_4_5 | 25 mer |
| 550 | GGAAGTAAAGCGGAACTGACCCTGT | SHANK3 H22A(+1712+1736) 2mm_4_7 | 25 mer |
| 551 | GGAGATGAAACGGAACTGACCCTGT | SHANK3 H22A(+1712+1736) 2mm_5_10 | 25 mer |
| 552 | GGAGATGAAGCAGAACTGACCCTGT | SHANK3 H22A(+1712+1736) 2mm_5_12 | 25 mer |
| 553 | GGAGATGAAGCGAAACTGACCCTGT | SHANK3 H22A(+1712+1736) 2mm_5_13 | 25 mer |
| 554 | GGAGATGAAGCGGAACTAACCCTGT | SHANK3 H22A(+1712+1736) 2mm_5_18 | 25 mer |
| 555 | GGAGATGAAGCGGAACTGACCCTAT | SHANK3 H22A(+1712+1736) 2mm_5_24 | 25 mer |
| 556 | GGAGATAAAGCGGAACTGACCCTGT | SHANK3 H22A(+1712+1736) 2mm_5_7 | 25 mer |
| 557 | GGAGGTAAAACGGAACTGACCCTGT | SHANK3 H22A(+1712+1736) 2mm_7_10 | 25 mer |
| 558 | GGAGGTAAAGCAGAACTGACCCTGT | SHANK3 H22A(+1712+1736) 2mm_7_12 | 25 mer |
| 559 | GGAGGTAAAGCGAAACTGACCCTGT | SHANK3 H22A(+1712+1736) 2mm_7_13 | 25 mer |
| 560 | GGAGGTAAAGCGGAACTAACCCTGT | SHANK3 H22A(+1712+1736) 2mm_7_18 | 25 mer |
| 561 | GGAGGTAAAGCGGAACTGACCCTAT | SHANK3 H22A(+1712+1736) 2mm_7_24 | 25 mer |
| 562 | GGAGGTGAAGCGGAACTGACCCTAT | SHANK3 H22A(+1712+1736) mm1 | 25 mer |
| 563 | AGGAGGTGAAGCGGAACTGACCCTG | SHANK3 H22A(+1713+1737) | 25 mer |
| 564 | AGGAGGTGAATCGGAACTGACCCTG | SHANK3 H22A(+1713+1737) 1mm_11 | 25 mer |
| 565 | AGGAGGTGAAGTGGAACTGACCCTG | SHANK3 H22A(+1713+1737) 1mm_12 | 25 mer |
| 566 | AGGAGGTGAAGCTGAACTGACCCTG | SHANK3 H22A(+1713+1737) 1mm_13 | 25 mer |
| 567 | AGGAGGTGAAGCGTAACTGACCCTG | SHANK3 H22A(+1713+1737) 1mm_14 | 25 mer |
| 568 | AGGAGGTGAAGCGGAATTGACCCTG | SHANK3 H22A(+1713+1737) 1mm_17 | 25 mer |
| 569 | AGGAGGTGAAGCGGAACTTACCCTG | SHANK3 H22A(+1713+1737) 1mm_19 | 25 mer |
| 570 | ATGAGGTGAAGCGGAACTGACCCTG | SHANK3 H22A(+1713+1737) 1mm_2 | 25 mer |
| 571 | AGGAGGTGAAGCGGAACTGATCCTG | SHANK3 H22A(+1713+1737) 1mm_21 | 25 mer |
| 572 | AGGAGGTGAAGCGGAACTGACTCTG | SHANK3 H22A(+1713+1737) 1mm_22 | 25 mer |
| 573 | AGGAGGTGAAGCGGAACTGACCTTG | SHANK3 H22A(+1713+1737) 1mm_23 | 25 mer |
| 574 | AGTAGGTGAAGCGGAACTGACCCTG | SHANK3 H22A(+1713+1737) 1mm_3 | 25 mer |
| 575 | AGGATGTGAAGCGGAACTGACCCTG | SHANK3 H22A(+1713+1737) 1mm_5 | 25 mer |
| 576 | AGGAGTTGAAGCGGAACTGACCCTG | SHANK3 H22A(+1713+1737) 1mm_6 | 25 mer |
| 577 | AGGAGGTTAAGCGGAACTGACCCTG | SHANK3 H22A(+1713+1737) 1mm_8 | 25 mer |
| 578 | AGGAGGTGAAACAGAACTGACCCTG | SHANK3 H22A(+1713+1737) 2mm_11_13 | 25 mer |
| 579 | AGGAGGTGAAACGAAACTGACCCTG | SHANK3 H22A(+1713+1737) 2mm_11_14 | 25 mer |
| 580 | AGGAGGTGAAACGGAACTAACCCTG | SHANK3 H22A(+1713+1737) 2mm_11_19 | 25 mer |
| 581 | AGGAGGTGAAACGGAACTGACCCTA | SHANK3 H22A(+1713+1737) 2mm_11_25 | 25 mer |
| 582 | AGGAGGTGAAGCAAAACTGACCCTG | SHANK3 H22A(+1713+1737) 2mm_13_14 | 25 mer |
| 583 | AGGAGGTGAAGCAGAACTAACCCTG | SHANK3 H22A(+1713+1737) 2mm_13_19 | 25 mer |
| 584 | AGGAGGTGAAGCAGAACTGACCCTA | SHANK3 H22A(+1713+1737) 2mm_13_25 | 25 mer |
| 585 | AGGAGGTGAAGCGAAACTAACCCTG | SHANK3 H22A(+1713+1737) 2mm_14_19 | 25 mer |
| 586 | AGGAGGTGAAGCGAAACTGACCCTA | SHANK3 H22A(+1713+1737) 2mm_14_25 | 25 mer |
| 587 | AAGAGGTGAAACGGAACTGACCCTG | SHANK3 H22A(+1713+1737) 2mm_2_11 | 25 mer |
| 588 | AAGAGGTGAAGCAGAACTGACCCTG | SHANK3 H22A(+1713+1737) 2mm_2_13 | 25 mer |
| 589 | AAGAGGTGAAGCGAAACTGACCCTG | SHANK3 H22A(+1713+1737) 2mm_2_14 | 25 mer |
| 590 | AAGAGGTGAAGCGGAACTAACCCTG | SHANK3 H22A(+1713+1737) 2mm_2_19 | 25 mer |
| 591 | AAGAGGTGAAGCGGAACTGACCCTA | SHANK3 H22A(+1713+1737) 2mm_2_25 | 25 mer |
| 592 | AAAAGGTGAAGCGGAACTGACCCTG | SHANK3 H22A(+1713+1737) 2mm_2_3 | 25 mer |
| 593 | AAGAAGTGAAGCGGAACTGACCCTG | SHANK3 H22A(+1713+1737) 2mm_2_5 | 25 mer |
| 594 | AAGAGATGAAGCGGAACTGACCCTG | SHANK3 H22A(+1713+1737) 2mm_2_6 | 25 mer |
| 595 | AAGAGGTAAAGCGGAACTGACCCTG | SHANK3 H22A(+1713+1737) 2mm_2_8 | 25 mer |
| 596 | AGAAGGTGAAACGGAACTGACCCTG | SHANK3 H22A(+1713+1737) 2mm_3_11 | 25 mer |
| 597 | AGAAGGTGAAGCAGAACTGACCCTG | SHANK3 H22A(+1713+1737) 2mm_3_13 | 25 mer |
| 598 | AGAAGGTGAAGCGAAACTGACCCTG | SHANK3 H22A(+1713+1737) 2mm_3_14 | 25 mer |
| 599 | AGAAGGTGAAGCGGAACTAACCCTG | SHANK3 H22A(+1713+1737) 2mm_3_19 | 25 mer |
| 600 | AGAAGGTGAAGCGGAACTGACCCTA | SHANK3 H22A(+1713+1737) 2mm_3_25 | 25 mer |
| 601 | AGAAAGTGAAGCGGAACTGACCCTG | SHANK3 H22A(+1713+1737) 2mm_3_5 | 25 mer |
| 602 | AGAAGATGAAGCGGAACTGACCCTG | SHANK3 H22A(+1713+1737) 2mm_3_6 | 25 mer |
| 603 | AGAAGGTAAAGCGGAACTGACCCTG | SHANK3 H22A(+1713+1737) 2mm_3_8 | 25 mer |
| 604 | AGGAAGTGAAACGGAACTGACCCTG | SHANK3 H22A(+1713+1737) 2mm_5_11 | 25 mer |
| 605 | AGGAAGTGAAGCAGAACTGACCCTG | SHANK3 H22A(+1713+1737) 2mm_5_13 | 25 mer |
| 606 | AGGAAGTGAAGCGAAACTGACCCTG | SHANK3 H22A(+1713+1737) 2mm_5_14 | 25 mer |
| 607 | AGGAAGTGAAGCGGAACTAACCCTG | SHANK3 H22A(+1713+1737) 2mm_5_19 | 25 mer |
| 608 | AGGAAGTGAAGCGGAACTGACCCTA | SHANK3 H22A(+1713+1737) 2mm_5_25 | 25 mer |
| 609 | AGGAAATGAAGCGGAACTGACCCTG | SHANK3 H22A(+1713+1737) 2mm_5_6 | 25 mer |
| 610 | AGGAAGTAAAGCGGAACTGACCCTG | SHANK3 H22A(+1713+1737) 2mm_5_8 | 25 mer |
| 611 | AGGAGATGAAACGGAACTGACCCTG | SHANK3 H22A(+1713+1737) 2mm_6_11 | 25 mer |
| 612 | AGGAGATGAAGCAGAACTGACCCTG | SHANK3 H22A(+1713+1737) 2mm_6_13 | 25 mer |
| 613 | AGGAGATGAAGCGAAACTGACCCTG | SHANK3 H22A(+1713+1737) 2mm_6_14 | 25 mer |
| 614 | AGGAGATGAAGCGGAACTAACCCTG | SHANK3 H22A(+1713+1737) 2mm_6_19 | 25 mer |
| 615 | AGGAGATGAAGCGGAACTGACCCTA | SHANK3 H22A(+1713+1737) 2mm_6_25 | 25 mer |
| 616 | AGGAGATAAAGCGGAACTGACCCTG | SHANK3 H22A(+1713+1737) 2mm_6_8 | 25 mer |
| 617 | AGGAGGTAAAACGGAACTGACCCTG | SHANK3 H22A(+1713+1737) 2mm_8_11 | 25 mer |
| 618 | AGGAGGTAAAGCAGAACTGACCCTG | SHANK3 H22A(+1713+1737) 2mm_8_13 | 25 mer |
| 619 | AGGAGGTAAAGCGAAACTGACCCTG | SHANK3 H22A(+1713+1737) 2mm_8_14 | 25 mer |
| 620 | AGGAGGTAAAGCGGAACTAACCCTG | SHANK3 H22A(+1713+1737) 2mm_8_19 | 25 mer |
| 621 | AGGAGGTAAAGCGGAACTGACCCTA | SHANK3 H22A(+1713+1737) 2mm_8_25 | 25 mer |
| 622 | AGGAGGTGAAGCGGAACTGACCCTA | SHANK3 H22A(+1713+1737) mm1 | 25 mer |
| 623 | AAGGAGGTGAATCGGAACTGACCCT | SHANK3 H22A(+1714+1738) 1mm_12 | 25 mer |
| 624 | AAGGAGGTGAAGTGGAACTGACCCT | SHANK3 H22A(+1714+1738) 1mm_13 | 25 mer |
| 625 | AAGGAGGTGAAGCTGAACTGACCCT | SHANK3 H22A(+1714+1738) 1mm_14 | 25 mer |
| 626 | AAGGAGGTGAAGCGTAACTGACCCT | SHANK3 H22A(+1714+1738) 1mm_15 | 25 mer |
| 627 | AAGGAGGTGAAGCGGAATTGACCCT | SHANK3 H22A(+1714+1738) 1mm_18 | 25 mer |
| 628 | AAGGAGGTGAAGCGGAACTTACCCT | SHANK3 H22A(+1714+1738) 1mm_20 | 25 mer |
| 629 | AAGGAGGTGAAGCGGAACTGATCCT | SHANK3 H22A(+1714+1738) 1mm_22 | 25 mer |
| 630 | AAGGAGGTGAAGCGGAACTGACTCT | SHANK3 H22A(+1714+1738) 1mm_23 | 25 mer |
| 631 | AAGGAGGTGAAGCGGAACTGACCTT | SHANK3 H22A(+1714+1738) 1mm_24 | 25 mer |
| 632 | AATGAGGTGAAGCGGAACTGACCCT | SHANK3 H22A(+1714+1738) 1mm_3 | 25 mer |
| 633 | AAGTAGGTGAAGCGGAACTGACCCT | SHANK3 H22A(+1714+1738) 1mm_4 | 25 mer |
| 634 | AAGGATGTGAAGCGGAACTGACCCT | SHANK3 H22A(+1714+1738) 1mm_6 | 25 mer |
| 635 | AAGGAGTTGAAGCGGAACTGACCCT | SHANK3 H22A(+1714+1738) 1mm_7 | 25 mer |
| 636 | AAGGAGGTTAAGCGGAACTGACCCT | SHANK3 H22A(+1714+1738) 1mm_9 | 25 mer |
| 637 | AAGGAGGTGAAGCGGAACTAACCCT | SHANK3 H22A(+1714+1738) mm1 | 25 mer |
| 638 | AAGGAGGTGAAGCGAAACTAACCCT | SHANK3 H22A(+1714+1738) mm2 | 25 mer |
| 639 | AAAGGAGGTGAAGCGGAACTGACCC | SHANK3 H22A(+1715+1739) | 25 mer |
| 640 | AAAGGAGGTTAAGCGGAACTGACCC | SHANK3 H22A(+1715+1739) 1mm_10 | 25 mer |
| 641 | AAAGGAGGTGAATCGGAACTGACCC | SHANK3 H22A(+1715+1739) 1mm_13 | 25 mer |
| 642 | AAAGGAGGTGAAGTGGAACTGACCC | SHANK3 H22A(+1715+1739) 1mm_14 | 25 mer |
| 643 | AAAGGAGGTGAAGCTGAACTGACCC | SHANK3 H22A(+1715+1739) 1mm_15 | 25 mer |
| 644 | AAAGGAGGTGAAGCGTAACTGACCC | SHANK3 H22A(+1715+1739) 1mm_16 | 25 mer |
| 645 | AAAGGAGGTGAAGCGGAATTGACCC | SHANK3 H22A(+1715+1739) 1mm_19 | 25 mer |
| 646 | AAAGGAGGTGAAGCGGAACTTACCC | SHANK3 H22A(+1715+1739) 1mm_21 | 25 mer |
| 647 | AAAGGAGGTGAAGCGGAACTGATCC | SHANK3 H22A(+1715+1739) 1mm_23 | 25 mer |
| 648 | AAAGGAGGTGAAGCGGAACTGACTC | SHANK3 H22A(+1715+1739) 1mm_24 | 25 mer |
| 649 | AAAGGAGGTGAAGCGGAACTGACCT | SHANK3 H22A(+1715+1739) 1mm_25 | 25 mer |
| 650 | AAATGAGGTGAAGCGGAACTGACCC | SHANK3 H22A(+1715+1739) 1mm_4 | 25 mer |
| 651 | AAAGTAGGTGAAGCGGAACTGACCC | SHANK3 H22A(+1715+1739) 1mm_5 | 25 mer |
| 652 | AAAGGATGTGAAGCGGAACTGACCC | SHANK3 H22A(+1715+1739) 1mm_7 | 25 mer |
| 653 | AAAGGAGTTGAAGCGGAACTGACCC | SHANK3 H22A(+1715+1739) 1mm_8 | 25 mer |
| 654 | AAAGGAGGTGAAGCGGAACTAACCC | SHANK3 H22A(+1715+1739) mm1 | 25 mer |
| 655 | AAAGGAGGTGAAGCGAAACTAACCC | SHANK3 H22A(+1715+1739) mm2 | 25 mer |
| 656 | AAAAGGAGGTGAAGCGGAACTGACC | SHANK3 H22A(+1716+1740) | 25 mer |
| 657 | AAAAGGAGGTTAAGCGGAACTGACC | SHANK3 H22A(+1716+1740) 1mm_11 | 25 mer |
| 658 | AAAAGGAGGTGAATCGGAACTGACC | SHANK3 H22A(+1716+1740) 1mm_14 | 25 mer |
| 659 | AAAAGGAGGTGAAGTGGAACTGACC | SHANK3 H22A(+1716+1740) 1mm_15 | 25 mer |
| 660 | AAAAGGAGGTGAAGCTGAACTGACC | SHANK3 H22A(+1716+1740) 1mm_16 | 25 mer |
| 661 | AAAAGGAGGTGAAGCGTAACTGACC | SHANK3 H22A(+1716+1740) 1mm_17 | 25 mer |
| 662 | AAAAGGAGGTGAAGCGGAATTGACC | SHANK3 H22A(+1716+1740) 1mm_20 | 25 mer |
| 663 | AAAAGGAGGTGAAGCGGAACTTACC | SHANK3 H22A(+1716+1740) 1mm_22 | 25 mer |
| 664 | AAAAGGAGGTGAAGCGGAACTGATC | SHANK3 H22A(+1716+1740) 1mm_24 | 25 mer |
| 665 | AAAAGGAGGTGAAGCGGAACTGACT | SHANK3 H22A(+1716+1740) 1mm_25 | 25 mer |
| 666 | AAAATGAGGTGAAGCGGAACTGACC | SHANK3 H22A(+1716+1740) 1mm_5 | 25 mer |
| 667 | AAAAGTAGGTGAAGCGGAACTGACC | SHANK3 H22A(+1716+1740) 1mm_6 | 25 mer |
| 668 | AAAAGGATGTGAAGCGGAACTGACC | SHANK3 H22A(+1716+1740) 1mm_8 | 25 mer |
| 669 | AAAAGGAGTTGAAGCGGAACTGACC | SHANK3 H22A(+1716+1740) 1mm_9 | 25 mer |
| 670 | AAAAGGAGGTGAAGCGGAACTAACC | SHANK3 H22A(+1716+1740) mm1 | 25 mer |
| 671 | AAAAGGAGGTGAAGCGAAACTAACC | SHANK3 H22A(+1716+1740) mm2 | 25 mer |
| 672 | TAAAAGGAGGTGAAGCGGAACTGAC | SHANK3 H22A(+1717+1741) 1mm_1 | 25 mer |
| 673 | CAAAAGGAGTTGAAGCGGAACTGAC | SHANK3 H22A(+1717+1741) 1mm_10 | 25 mer |
| 674 | CAAAAGGAGGTTAAGCGGAACTGAC | SHANK3 H22A(+1717+1741) 1mm_12 | 25 mer |
| 675 | CAAAAGGAGGTGAATCGGAACTGAC | SHANK3 H22A(+1717+1741) 1mm_15 | 25 mer |
| 676 | CAAAAGGAGGTGAAGTGGAACTGAC | SHANK3 H22A(+1717+1741) 1mm_16 | 25 mer |
| 677 | CAAAAGGAGGTGAAGCTGAACTGAC | SHANK3 H22A(+1717+1741) 1mm_17 | 25 mer |
| 678 | CAAAAGGAGGTGAAGCGTAACTGAC | SHANK3 H22A(+1717+1741) 1mm_18 | 25 mer |
| 679 | CAAAAGGAGGTGAAGCGGAATTGAC | SHANK3 H22A(+1717+1741) 1mm_21 | 25 mer |
| 680 | CAAAAGGAGGTGAAGCGGAACTTAC | SHANK3 H22A(+1717+1741) 1mm_23 | 25 mer |
| 681 | CAAAAGGAGGTGAAGCGGAACTGAT | SHANK3 H22A(+1717+1741) 1mm_25 | 25 mer |
| 682 | CAAAATGAGGTGAAGCGGAACTGAC | SHANK3 H22A(+1717+1741) 1mm_6 | 25 mer |
| 683 | CAAAAGTAGGTGAAGCGGAACTGAC | SHANK3 H22A(+1717+1741) 1mm_7 | 25 mer |
| 684 | CAAAAGGATGTGAAGCGGAACTGAC | SHANK3 H22A(+1717+1741) 1mm_9 | 25 mer |
| 685 | CAAAAGGAGGTGAAGCGGAACTAAC | SHANK3 H22A(+1717+1741) mm1 | 25 mer |
| 686 | CAAAAGGAGGTGAAGCGAAACTAAC | SHANK3 H22A(+1717+1741) mm2 | 25 mer |
| 687 | ACAAAAGGAGGTGAAGCGGAACTGA | SHANK3 H22A(+1718+1742) | 25 mer |
| 688 | ACAAAAGGATGTGAAGCGGAACTGA | SHANK3 H22A(+1718+1742) 1mm_10 | 25 mer |
| 689 | ACAAAAGGAGTTGAAGCGGAACTGA | SHANK3 H22A(+1718+1742) 1mm_11 | 25 mer |
| 690 | ACAAAAGGAGGTTAAGCGGAACTGA | SHANK3 H22A(+1718+1742) 1mm_13 | 25 mer |
| 691 | ACAAAAGGAGGTGAATCGGAACTGA | SHANK3 H22A(+1718+1742) 1mm_16 | 25 mer |
| 692 | ACAAAAGGAGGTGAAGTGGAACTGA | SHANK3 H22A(+1718+1742) 1mm_17 | 25 mer |
| 693 | ACAAAAGGAGGTGAAGCTGAACTGA | SHANK3 H22A(+1718+1742) 1mm_18 | 25 mer |
| 694 | ACAAAAGGAGGTGAAGCGTAACTGA | SHANK3 H22A(+1718+1742) 1mm_19 | 25 mer |
| 695 | ATAAAAGGAGGTGAAGCGGAACTGA | SHANK3 H22A(+1718+1742) 1mm_2 | 25 mer |
| 696 | ACAAAAGGAGGTGAAGCGGAATTGA | SHANK3 H22A(+1718+1742) 1mm_22 | 25 mer |
| 697 | ACAAAAGGAGGTGAAGCGGAACTTA | SHANK3 H22A(+1718+1742) 1mm_24 | 25 mer |
| 698 | ACAAAATGAGGTGAAGCGGAACTGA | SHANK3 H22A(+1718+1742) 1mm_7 | 25 mer |
| 699 | ACAAAAGTAGGTGAAGCGGAACTGA | SHANK3 H22A(+1718+1742) 1mm_8 | 25 mer |
| 700 | ACAAAAGGAGGTGAAGCGGAACTAA | SHANK3 H22A(+1718+1742) mm1 | 25 mer |
| 701 | ACAAAAGGAGGTGAAGCGAAACTAA | SHANK3 H22A(+1718+1742) mm2 | 25 mer |
| 702 | TACAAAAGGAGGTGAAGCGGAACTG | SHANK3 H22A(+1719+1743) | 25 mer |
| 703 | TACAAAAGGATGTGAAGCGGAACTG | SHANK3 H22A(+1719+1743) 1mm_11 | 25 mer |
| 704 | TACAAAAGGAGTTGAAGCGGAACTG | SHANK3 H22A(+1719+1743) 1mm_12 | 25 mer |
| 705 | TACAAAAGGAGGTTAAGCGGAACTG | SHANK3 H22A(+1719+1743) 1mm_14 | 25 mer |
| 706 | TACAAAAGGAGGTGAATCGGAACTG | SHANK3 H22A(+1719+1743) 1mm_17 | 25 mer |
| 707 | TACAAAAGGAGGTGAAGTGGAACTG | SHANK3 H22A(+1719+1743) 1mm_18 | 25 mer |
| 708 | TACAAAAGGAGGTGAAGCTGAACTG | SHANK3 H22A(+1719+1743) 1mm_19 | 25 mer |
| 709 | TACAAAAGGAGGTGAAGCGTAACTG | SHANK3 H22A(+1719+1743) 1mm_20 | 25 mer |
| 710 | TACAAAAGGAGGTGAAGCGGAATTG | SHANK3 H22A(+1719+1743) 1mm_23 | 25 mer |
| 711 | TATAAAAGGAGGTGAAGCGGAACTG | SHANK3 H22A(+1719+1743) 1mm_3 | 25 mer |
| 712 | TACAAAATGAGGTGAAGCGGAACTG | SHANK3 H22A(+1719+1743) 1mm_8 | 25 mer |
| 713 | TACAAAAGTAGGTGAAGCGGAACTG | SHANK3 H22A(+1719+1743) 1mm_9 | 25 mer |
| 714 | TACAAAAGGAGGTGAAGCGGAACTA | SHANK3 H22A(+1719+1743) mm1 | 25 mer |
| 715 | TACAAAAGGAGGTGAAGCGAAACTA | SHANK3 H22A(+1719+1743) mm2 | 25 mer |
| 716 | ATACAAAAGTAGGTGAAGCGGAACT | SHANK3 H22A(+1720+1744) 1mm_10 | 25 mer |
| 717 | ATACAAAAGGATGTGAAGCGGAACT | SHANK3 H22A(+1720+1744) 1mm_12 | 25 mer |
| 718 | ATACAAAAGGAGTTGAAGCGGAACT | SHANK3 H22A(+1720+1744) 1mm_13 | 25 mer |
| 719 | ATACAAAAGGAGGTTAAGCGGAACT | SHANK3 H22A(+1720+1744) 1mm_15 | 25 mer |
| 720 | ATACAAAAGGAGGTGAATCGGAACT | SHANK3 H22A(+1720+1744) 1mm_18 | 25 mer |
| 721 | ATACAAAAGGAGGTGAAGTGGAACT | SHANK3 H22A(+1720+1744) 1mm_19 | 25 mer |
| 722 | ATACAAAAGGAGGTGAAGCTGAACT | SHANK3 H22A(+1720+1744) 1mm_20 | 25 mer |
| 723 | ATACAAAAGGAGGTGAAGCGTAACT | SHANK3 H22A(+1720+1744) 1mm_21 | 25 mer |
| 724 | ATACAAAAGGAGGTGAAGCGGAATT | SHANK3 H22A(+1720+1744) 1mm_24 | 25 mer |
| 725 | ATATAAAAGGAGGTGAAGCGGAACT | SHANK3 H22A(+1720+1744) 1mm_4 | 25 mer |
| 726 | ATACAAAATGAGGTGAAGCGGAACT | SHANK3 H22A(+1720+1744) 1mm_9 | 25 mer |
| 727 | ATACAAAAGGAGGTGAAACGAAACT | SHANK3 H22A(+1720+1744) 2mm_18_21 | 25 mer |
| 728 | ATACAAAAGGAGGTGAAGCGAAACT | SHANK3 H22A(+1720+1744) mm1 | 25 mer |
| 729 | ATACAAAAGGAGGTGAAGCAAAACT | SHANK3 H22A(+1720+1744) mm2 | 25 mer |
| 730 | TAAAAACCCAAATTTAAC | SHANK3 H22A(+1689+1706) 1mm_15 | 18 mer |
| 731 | TAAAAACCCAAATTGAAT | SHANK3 H22A(+1689+1706) 1mm_18 | 18 mer |
| 732 | TAAAAATCCAAATTGAAC | SHANK3 H22A(+1689+1706) 1mm_7 | 18 mer |
| 733 | TAAAAACTCAAATTGAAC | SHANK3 H22A(+1689+1706) 1mm_8 | 18 mer |
| 734 | TAAAAACCTAAATTGAAC | SHANK3 H22A(+1689+1706) 1mm_9 | 18 mer |
| 735 | TTAAAAACCTAAATTGAA | SHANK3 H22A(+1690+1707) 1mm_10 | 18 mer |
| 736 | TTAAAAACCCAAATTTAA | SHANK3 H22A(+1690+1707) 1mm_16 | 18 mer |
| 737 | TTAAAAATCCAAATTGAA | SHANK3 H22A(+1690+1707) 1mm_8 | 18 mer |
| 738 | TTAAAAACTCAAATTGAA | SHANK3 H22A(+1690+1707) 1mm_9 | 18 mer |
| 739 | TTTAAAAACCCAAATTGA | SHANK3 H22A(+1691+1708) 1mm_1 | 18 mer |
| 740 | GTTAAAAACTCAAATTGA | SHANK3 H22A(+1691+1708) 1mm_10 | 18 mer |
| 741 | GTTAAAAACCTAAATTGA | SHANK3 H22A(+1691+1708) 1mm_11 | 18 mer |
| 742 | GTTAAAAACCCAAATTTA | SHANK3 H22A(+1691+1708) 1mm_17 | 18 mer |
| 743 | GTTAAAAATCCAAATTGA | SHANK3 H22A(+1691+1708) 1mm_9 | 18 mer |
| 744 | AGTTAAAAATCCAAATTG | SHANK3 H22A(+1692+1709) 1mm_10 | 18 mer |
| 745 | AGTTAAAAACTCAAATTG | SHANK3 H22A(+1692+1709) 1mm_11 | 18 mer |
| 746 | AGTTAAAAACCTAAATTG | SHANK3 H22A(+1692+1709) 1mm_12 | 18 mer |
| 747 | AGTTAAAAACCCAAATTT | SHANK3 H22A(+1692+1709) 1mm_18 | 18 mer |
| 748 | ATTTAAAAACCCAAATTG | SHANK3 H22A(+1692+1709) 1mm_2 | 18 mer |
| 749 | AAGTTAAAAATCCAAATT | SHANK3 H22A(+1693+1710) 1mm_11 | 18 mer |
| 750 | AAGTTAAAAACTCAAATT | SHANK3 H22A(+1693+1710) 1mm_12 | 18 mer |
| 751 | AAGTTAAAAACCTAAATT | SHANK3 H22A(+1693+1710) 1mm_13 | 18 mer |
| 752 | AATTTAAAAACCCAAATT | SHANK3 H22A(+1693+1710) 1mm_3 | 18 mer |
| 753 | AAAGTTAAAAATCCAAAT | SHANK3 H22A(+1694+1711) 1mm_12 | 18 mer |
| 754 | AAAGTTAAAAACTCAAAT | SHANK3 H22A(+1694+1711) 1mm_13 | 18 mer |
| 755 | AAAGTTAAAAACCTAAAT | SHANK3 H22A(+1694+1711) 1mm_14 | 18 mer |
| 756 | AAATTTAAAAACCCAAAT | SHANK3 H22A(+1694+1711) 1mm_4 | 18 mer |
| 757 | TAAAGTTAAAAATCCAAA | SHANK3 H22A(+1695+1712) 1mm_13 | 18 mer |
| 758 | TAAAGTTAAAAACTCAAA | SHANK3 H22A(+1695+1712) 1mm_14 | 18 mer |
| 759 | TAAAGTTAAAAACCTAAA | SHANK3 H22A(+1695+1712) 1mm_15 | 18 mer |
| 760 | TAAATTTAAAAACCCAAA | SHANK3 H22A(+1695+1712) 1mm_5 | 18 mer |
| 761 | TTAAAGTTAAAAACCCAA | SHANK3 H22A(+1696+1713) 1mm_1 | 18 mer |
| 762 | GTAAAGTTAAAAATCCAA | SHANK3 H22A(+1696+1713) 1mm_14 | 18 mer |
| 763 | GTAAAGTTAAAAACTCAA | SHANK3 H22A(+1696+1713) 1mm_15 | 18 mer |
| 764 | GTAAAGTTAAAAACCTAA | SHANK3 H22A(+1696+1713) 1mm_16 | 18 mer |
| 765 | GTAAATTTAAAAACCCAA | SHANK3 H22A(+1696+1713) 1mm_6 | 18 mer |
| 766 | TGTAAAGTTAAAAATCCA | SHANK3 H22A(+1697+1714) 1mm_15 | 18 mer |
| 767 | TGTAAAGTTAAAAACTCA | SHANK3 H22A(+1697+1714) 1mm_16 | 18 mer |
| 768 | TGTAAAGTTAAAAACCTA | SHANK3 H22A(+1697+1714) 1mm_17 | 18 mer |
| 769 | TTTAAAGTTAAAAACCCA | SHANK3 H22A(+1697+1714) 1mm_2 | 18 mer |
| 770 | TGTAAATTTAAAAACCCA | SHANK3 H22A(+1697+1714) 1mm_7 | 18 mer |
| 771 | TTGTAAAGTTAAAAACCC | SHANK3 H22A(+1698+1715) 1mm_1 | 18 mer |
| 772 | CTGTAAAGTTAAAAATCC | SHANK3 H22A(+1698+1715) 1mm_16 | 18 mer |
| 773 | CTGTAAAGTTAAAAACTC | SHANK3 H22A(+1698+1715) 1mm_17 | 18 mer |
| 774 | CTGTAAAGTTAAAAACCT | SHANK3 H22A(+1698+1715) 1mm_18 | 18 mer |
| 775 | CTTTAAAGTTAAAAACCC | SHANK3 H22A(+1698+1715) 1mm_3 | 18 mer |
| 776 | CTGTAAATTTAAAAACCC | SHANK3 H22A(+1698+1715) 1mm_8 | 18 mer |
| 777 | TCTGTAAAGTTAAAAACC | SHANK3 H22A(+1699+1716) 1mm_1 | 18 mer |
| 778 | CCTGTAAAGTTAAAAATC | SHANK3 H22A(+1699+1716) 1mm_17 | 18 mer |
| 779 | CCTGTAAAGTTAAAAACT | SHANK3 H22A(+1699+1716) 1mm_18 | 18 mer |
| 780 | CTTGTAAAGTTAAAAACC | SHANK3 H22A(+1699+1716) 1mm_2 | 18 mer |
| 781 | CCTTTAAAGTTAAAAACC | SHANK3 H22A(+1699+1716) 1mm_4 | 18 mer |
| 782 | CCTGTAAATTTAAAAACC | SHANK3 H22A(+1699+1716) 1mm_9 | 18 mer |
| 783 | TCCTGTAAAGTTAAAAAC | SHANK3 H22A(+1700+1717) 1mm_1 | 18 mer |
| 784 | CCCTGTAAATTTAAAAAC | SHANK3 H22A(+1700+1717) 1mm_10 | 18 mer |
| 785 | CCCTGTAAAGTTAAAAAT | SHANK3 H22A(+1700+1717) 1mm_18 | 18 mer |
| 786 | CTCTGTAAAGTTAAAAAC | SHANK3 H22A(+1700+1717) 1mm_2 | 18 mer |
| 787 | CCTTGTAAAGTTAAAAAC | SHANK3 H22A(+1700+1717) 1mm_3 | 18 mer |
| 788 | CCCTTTAAAGTTAAAAAC | SHANK3 H22A(+1700+1717) 1mm_5 | 18 mer |
| 789 | ACCCTGTAAATTTAAAAA | SHANK3 H22A(+1701+1718) 1mm_11 | 18 mer |
| 790 | ATCCTGTAAAGTTAAAAA | SHANK3 H22A(+1701+1718) 1mm_2 | 18 mer |
| 791 | ACTCTGTAAAGTTAAAAA | SHANK3 H22A(+1701+1718) 1mm_3 | 18 mer |
| 792 | ACCTTGTAAAGTTAAAAA | SHANK3 H22A(+1701+1718) 1mm_4 | 18 mer |
| 793 | ACCCTTTAAAGTTAAAAA | SHANK3 H22A(+1701+1718) 1mm_6 | 18 mer |
| 794 | TACCCTGTAAAGTTAAAA | SHANK3 H22A(+1702+1719) 1mm_1 | 18 mer |
| 795 | GACCCTGTAAATTTAAAA | SHANK3 H22A(+1702+1719) 1mm_12 | 18 mer |
| 796 | GATCCTGTAAAGTTAAAA | SHANK3 H22A(+1702+1719) 1mm_3 | 18 mer |
| 797 | GACTCTGTAAAGTTAAAA | SHANK3 H22A(+1702+1719) 1mm_4 | 18 mer |
| 798 | GACCTTGTAAAGTTAAAA | SHANK3 H22A(+1702+1719) 1mm_5 | 18 mer |
| 799 | GACCCTTTAAAGTTAAAA | SHANK3 H22A(+1702+1719) 1mm_7 | 18 mer |
| 800 | TGACCCTGTAAAGTTAAA | SHANK3 H22A(+1703+1720) | 18 mer |
| 801 | TGACCCTGTAAATTTAAA | SHANK3 H22A(+1703+1720) 1mm_13 | 18 mer |
| 802 | TTACCCTGTAAAGTTAAA | SHANK3 H22A(+1703+1720) 1mm_2 | 18 mer |
| 803 | TGATCCTGTAAAGTTAAA | SHANK3 H22A(+1703+1720) 1mm_4 | 18 mer |
| 804 | TGACTCTGTAAAGTTAAA | SHANK3 H22A(+1703+1720) 1mm_5 | 18 mer |
| 805 | TGACCTTGTAAAGTTAAA | SHANK3 H22A(+1703+1720) 1mm_6 | 18 mer |
| 806 | TGACCCTTTAAAGTTAAA | SHANK3 H22A(+1703+1720) 1mm_8 | 18 mer |
| 807 | TGACCCTGTAAAATTAAA | SHANK3 H22A(+1703+1720) mm1 | 18 mer |
| 808 | CTGACCCTGTAAAGTTAA | SHANK3 H22A(+1704+1721) | 18 mer |
| 809 | TTGACCCTGTAAAGTTAA | SHANK3 H22A(+1704+1721) 1mm_1 | 18 mer |
| 810 | CTGACCCTGTAAATTTAA | SHANK3 H22A(+1704+1721) 1mm_14 | 18 mer |
| 811 | CTTACCCTGTAAAGTTAA | SHANK3 H22A(+1704+1721) 1mm_3 | 18 mer |
| 812 | CTGATCCTGTAAAGTTAA | SHANK3 H22A(+1704+1721) 1mm_5 | 18 mer |
| 813 | CTGACTCTGTAAAGTTAA | SHANK3 H22A(+1704+1721) 1mm_6 | 18 mer |
| 814 | CTGACCTTGTAAAGTTAA | SHANK3 H22A(+1704+1721) 1mm_7 | 18 mer |
| 815 | CTGACCCTTTAAAGTTAA | SHANK3 H22A(+1704+1721) 1mm_9 | 18 mer |
| 816 | CTGACCCTGTAAAATTAA | SHANK3 H22A(+1704+1721) mm1 | 18 mer |
| 817 | ACTGACCCTGTAAAGTTA | SHANK3 H22A(+1705+1722) | 18 mer |
| 818 | ACTGACCCTTTAAAGTTA | SHANK3 H22A(+1705+1722) 1mm_10 | 18 mer |
| 819 | ACTGACCCTGTAAATTTA | SHANK3 H22A(+1705+1722) 1mm_15 | 18 mer |
| 820 | ATTGACCCTGTAAAGTTA | SHANK3 H22A(+1705+1722) 1mm_2 | 18 mer |
| 821 | ACTTACCCTGTAAAGTTA | SHANK3 H22A(+1705+1722) 1mm_4 | 18 mer |
| 822 | ACTGATCCTGTAAAGTTA | SHANK3 H22A(+1705+1722) 1mm_6 | 18 mer |
| 823 | ACTGACTCTGTAAAGTTA | SHANK3 H22A(+1705+1722) 1mm_7 | 18 mer |
| 824 | ACTGACCTTGTAAAGTTA | SHANK3 H22A(+1705+1722) 1mm_8 | 18 mer |
| 825 | ACTGACCCTGTAAAATTA | SHANK3 H22A(+1705+1722) mm1 | 18 mer |
| 826 | AACTGACCCTGTAAAGTT | SHANK3 H22A(+1706+1723) | 18 mer |
| 827 | AACTGACCCTTTAAAGTT | SHANK3 H22A(+1706+1723) 1mm_11 | 18 mer |
| 828 | AACTGACCCTGTAAATTT | SHANK3 H22A(+1706+1723) 1mm_16 | 18 mer |
| 829 | AATTGACCCTGTAAAGTT | SHANK3 H22A(+1706+1723) 1mm_3 | 18 mer |
| 830 | AACTTACCCTGTAAAGTT | SHANK3 H22A(+1706+1723) 1mm_5 | 18 mer |
| 831 | AACTGATCCTGTAAAGTT | SHANK3 H22A(+1706+1723) 1mm_7 | 18 mer |
| 832 | AACTGACTCTGTAAAGTT | SHANK3 H22A(+1706+1723) 1mm_8 | 18 mer |
| 833 | AACTGACCTTGTAAAGTT | SHANK3 H22A(+1706+1723) 1mm_9 | 18 mer |
| 834 | AACTGACCCTGTAAAATT | SHANK3 H22A(+1706+1723) mm1 | 18 mer |
| 835 | GAACTGACCCTGTAAAGT | SHANK3 H22A(+1707+1724) | 18 mer |
| 836 | TAACTGACCCTGTAAAGT | SHANK3 H22A(+1707+1724) 1mm_1 | 18 mer |
| 837 | GAACTGACCTTGTAAAGT | SHANK3 H22A(+1707+1724) 1mm_10 | 18 mer |
| 838 | GAACTGACCCTTTAAAGT | SHANK3 H22A(+1707+1724) 1mm_12 | 18 mer |
| 839 | GAACTGACCCTGTAAATT | SHANK3 H22A(+1707+1724) 1mm_17 | 18 mer |
| 840 | GAATTGACCCTGTAAAGT | SHANK3 H22A(+1707+1724) 1mm_4 | 18 mer |
| 841 | GAACTTACCCTGTAAAGT | SHANK3 H22A(+1707+1724) 1mm_6 | 18 mer |
| 842 | GAACTGATCCTGTAAAGT | SHANK3 H22A(+1707+1724) 1mm_8 | 18 mer |
| 843 | GAACTGACTCTGTAAAGT | SHANK3 H22A(+1707+1724) 1mm_9 | 18 mer |
| 844 | GAACTGACCCTGTAAAAT | SHANK3 H22A(+1707+1724) mm1 | 18 mer |
| 845 | GGAACTGACCCTGTAAAG | SHANK3 H22A(+1708+1725) | 18 mer |
| 846 | TGAACTGACCCTGTAAAG | SHANK3 H22A(+1708+1725) 1mm_1 | 18 mer |
| 847 | GGAACTGACTCTGTAAAG | SHANK3 H22A(+1708+1725) 1mm_10 | 18 mer |
| 848 | GGAACTGACCTTGTAAAG | SHANK3 H22A(+1708+1725) 1mm_11 | 18 mer |
| 849 | GGAACTGACCCTTTAAAG | SHANK3 H22A(+1708+1725) 1mm_13 | 18 mer |
| 850 | GGAACTGACCCTGTAAAT | SHANK3 H22A(+1708+1725) 1mm_18 | 18 mer |
| 851 | GTAACTGACCCTGTAAAG | SHANK3 H22A(+1708+1725) 1mm_2 | 18 mer |
| 852 | GGAATTGACCCTGTAAAG | SHANK3 H22A(+1708+1725) 1mm_5 | 18 mer |
| 853 | GGAACTTACCCTGTAAAG | SHANK3 H22A(+1708+1725) 1mm_7 | 18 mer |
| 854 | GGAACTGATCCTGTAAAG | SHANK3 H22A(+1708+1725) 1mm_9 | 18 mer |
| 855 | GGAACTGACCCTGTAAAA | SHANK3 H22A(+1708+1725) mm1 | 18 mer |
| 856 | CGGAACTGACCCTGTAAA | SHANK3 H22A(+1709+1726) | 18 mer |
| 857 | TGGAACTGACCCTGTAAA | SHANK3 H22A(+1709+1726) 1mm_1 | 18 mer |
| 858 | CGGAACTGATCCTGTAAA | SHANK3 H22A(+1709+1726) 1mm_10 | 18 mer |
| 859 | CGGAACTGACTCTGTAAA | SHANK3 H22A(+1709+1726) 1mm_11 | 18 mer |
| 860 | CGGAACTGACCTTGTAAA | SHANK3 H22A(+1709+1726) 1mm_12 | 18 mer |
| 861 | CGGAACTGACCCTTTAAA | SHANK3 H22A(+1709+1726) 1mm_14 | 18 mer |
| 862 | CTGAACTGACCCTGTAAA | SHANK3 H22A(+1709+1726) 1mm_2 | 18 mer |
| 863 | CGTAACTGACCCTGTAAA | SHANK3 H22A(+1709+1726) 1mm_3 | 18 mer |
| 864 | CGGAATTGACCCTGTAAA | SHANK3 H22A(+1709+1726) 1mm_6 | 18 mer |
| 865 | CGGAACTTACCCTGTAAA | SHANK3 H22A(+1709+1726) 1mm_8 | 18 mer |
| 866 | CGGAACTGACCCTATAAA | SHANK3 H22A(+1709+1726) mm1 | 18 mer |
| 867 | GCGGAACTGACCCTGTAA | SHANK3 H22A(+1710+1727) | 18 mer |
| 868 | TCGGAACTGACCCTGTAA | SHANK3 H22A(+1710+1727) 1mm_1 | 18 mer |
| 869 | GCGGAACTGATCCTGTAA | SHANK3 H22A(+1710+1727) 1mm_11 | 18 mer |
| 870 | GCGGAACTGACTCTGTAA | SHANK3 H22A(+1710+1727) 1mm_12 | 18 mer |
| 871 | GCGGAACTGACCTTGTAA | SHANK3 H22A(+1710+1727) 1mm_13 | 18 mer |
| 872 | GCGGAACTGACCCTTTAA | SHANK3 H22A(+1710+1727) 1mm_15 | 18 mer |
| 873 | GTGGAACTGACCCTGTAA | SHANK3 H22A(+1710+1727) 1mm_2 | 18 mer |
| 874 | GCTGAACTGACCCTGTAA | SHANK3 H22A(+1710+1727) 1mm_3 | 18 mer |
| 875 | GCGTAACTGACCCTGTAA | SHANK3 H22A(+1710+1727) 1mm_4 | 18 mer |
| 876 | GCGGAATTGACCCTGTAA | SHANK3 H22A(+1710+1727) 1mm_7 | 18 mer |
| 877 | GCGGAACTTACCCTGTAA | SHANK3 H22A(+1710+1727) 1mm_9 | 18 mer |
| 878 | GCGGAACTGACCCTATAA | SHANK3 H22A(+1710+1727) mm1 | 18 mer |
| 879 | AGCGGAACTGACCCTGTA | SHANK3 H22A(+1711+1728) | 18 mer |
| 880 | AGCGGAACTTACCCTGTA | SHANK3 H22A(+1711+1728) 1mm_10 | 18 mer |
| 881 | AGCGGAACTGATCCTGTA | SHANK3 H22A(+1711+1728) 1mm_12 | 18 mer |
| 882 | AGCGGAACTGACTCTGTA | SHANK3 H22A(+1711+1728) 1mm_13 | 18 mer |
| 883 | AGCGGAACTGACCTTGTA | SHANK3 H22A(+1711+1728) 1mm_14 | 18 mer |
| 884 | AGCGGAACTGACCCTTTA | SHANK3 H22A(+1711+1728) 1mm_16 | 18 mer |
| 885 | ATCGGAACTGACCCTGTA | SHANK3 H22A(+1711+1728) 1mm_2 | 18 mer |
| 886 | AGTGGAACTGACCCTGTA | SHANK3 H22A(+1711+1728) 1mm_3 | 18 mer |
| 887 | AGCTGAACTGACCCTGTA | SHANK3 H22A(+1711+1728) 1mm_4 | 18 mer |
| 888 | AGCGTAACTGACCCTGTA | SHANK3 H22A(+1711+1728) 1mm_5 | 18 mer |
| 889 | AGCGGAATTGACCCTGTA | SHANK3 H22A(+1711+1728) 1mm_8 | 18 mer |
| 890 | AGCGGAACTGACCCTATA | SHANK3 H22A(+1711+1728) mm1 | 18 mer |
| 891 | AAGCGGAACTGACCCTGT | SHANK3 H22A(+1712+1729) | 18 mer |
| 892 | AAGCGGAACTTACCCTGT | SHANK3 H22A(+1712+1729) 1mm_11 | 18 mer |
| 893 | AAGCGGAACTGATCCTGT | SHANK3 H22A(+1712+1729) 1mm_13 | 18 mer |
| 894 | AAGCGGAACTGACTCTGT | SHANK3 H22A(+1712+1729) 1mm_14 | 18 mer |
| 895 | AAGCGGAACTGACCTTGT | SHANK3 H22A(+1712+1729) 1mm_15 | 18 mer |
| 896 | AAGCGGAACTGACCCTTT | SHANK3 H22A(+1712+1729) 1mm_17 | 18 mer |
| 897 | AATCGGAACTGACCCTGT | SHANK3 H22A(+1712+1729) 1mm_3 | 18 mer |
| 898 | AAGTGGAACTGACCCTGT | SHANK3 H22A(+1712+1729) 1mm_4 | 18 mer |
| 899 | AAGCTGAACTGACCCTGT | SHANK3 H22A(+1712+1729) 1mm_5 | 18 mer |
| 900 | AAGCGTAACTGACCCTGT | SHANK3 H22A(+1712+1729) 1mm_6 | 18 mer |
| 901 | AAGCGGAATTGACCCTGT | SHANK3 H22A(+1712+1729) 1mm_9 | 18 mer |
| 902 | AAGCGGAACTGACCCTAT | SHANK3 H22A(+1712+1729) mm1 | 18 mer |
| 903 | GAAGCGGAACTGACCCTG | SHANK3 H22A(+1713+1730) | 18 mer |
| 904 | TAAGCGGAACTGACCCTG | SHANK3 H22A(+1713+1730) 1mm_1 | 18 mer |
| 905 | GAAGCGGAATTGACCCTG | SHANK3 H22A(+1713+1730) 1mm_10 | 18 mer |
| 906 | GAAGCGGAACTTACCCTG | SHANK3 H22A(+1713+1730) 1mm_12 | 18 mer |
| 907 | GAAGCGGAACTGATCCTG | SHANK3 H22A(+1713+1730) 1mm_14 | 18 mer |
| 908 | GAAGCGGAACTGACTCTG | SHANK3 H22A(+1713+1730) 1mm_15 | 18 mer |
| 909 | GAAGCGGAACTGACCTTG | SHANK3 H22A(+1713+1730) 1mm_16 | 18 mer |
| 910 | GAAGCGGAACTGACCCTT | SHANK3 H22A(+1713+1730) 1mm_18 | 18 mer |
| 911 | GAATCGGAACTGACCCTG | SHANK3 H22A(+1713+1730) 1mm_4 | 18 mer |
| 912 | GAAGTGGAACTGACCCTG | SHANK3 H22A(+1713+1730) 1mm_5 | 18 mer |
| 913 | GAAGCTGAACTGACCCTG | SHANK3 H22A(+1713+1730) 1mm_6 | 18 mer |
| 914 | GAAGCGTAACTGACCCTG | SHANK3 H22A(+1713+1730) 1mm_7 | 18 mer |
| 915 | GAAGCGGAACTGACCCTA | SHANK3 H22A(+1713+1730) mm1 | 18 mer |
| 916 | TGAAGCGGAACTGACCCT | SHANK3 H22A(+1714+1731) | 18 mer |
| 917 | TGAAGCGGAATTGACCCT | SHANK3 H22A(+1714+1731) 1mm_11 | 18 mer |
| 918 | TGAAGCGGAACTTACCCT | SHANK3 H22A(+1714+1731) 1mm_13 | 18 mer |
| 919 | TGAAGCGGAACTGATCCT | SHANK3 H22A(+1714+1731) 1mm_15 | 18 mer |
| 920 | TGAAGCGGAACTGACTCT | SHANK3 H22A(+1714+1731) 1mm_16 | 18 mer |
| 921 | TGAAGCGGAACTGACCTT | SHANK3 H22A(+1714+1731) 1mm_17 | 18 mer |
| 922 | TTAAGCGGAACTGACCCT | SHANK3 H22A(+1714+1731) 1mm_2 | 18 mer |
| 923 | TGAATCGGAACTGACCCT | SHANK3 H22A(+1714+1731) 1mm_5 | 18 mer |
| 924 | TGAAGTGGAACTGACCCT | SHANK3 H22A(+1714+1731) 1mm_6 | 18 mer |
| 925 | TGAAGCTGAACTGACCCT | SHANK3 H22A(+1714+1731) 1mm_7 | 18 mer |
| 926 | TGAAGCGTAACTGACCCT | SHANK3 H22A(+1714+1731) 1mm_8 | 18 mer |
| 927 | TGAAGCGGAACTAACCCT | SHANK3 H22A(+1714+1731) mm1 | 18 mer |
| 928 | GTGAAGCGGAACTGACCC | SHANK3 H22A(+1715+1732) | 18 mer |
| 929 | TTGAAGCGGAACTGACCC | SHANK3 H22A(+1715+1732) 1mm_1 | 18 mer |
| 930 | GTGAAGCGGAATTGACCC | SHANK3 H22A(+1715+1732) 1mm_12 | 18 mer |
| 931 | GTGAAGCGGAACTTACCC | SHANK3 H22A(+1715+1732) 1mm_14 | 18 mer |
| 932 | GTGAAGCGGAACTGATCC | SHANK3 H22A(+1715+1732) 1mm_16 | 18 mer |
| 933 | GTGAAGCGGAACTGACTC | SHANK3 H22A(+1715+1732) 1mm_17 | 18 mer |
| 934 | GTGAAGCGGAACTGACCT | SHANK3 H22A(+1715+1732) 1mm_18 | 18 mer |
| 935 | GTTAAGCGGAACTGACCC | SHANK3 H22A(+1715+1732) 1mm_3 | 18 mer |
| 936 | GTGAATCGGAACTGACCC | SHANK3 H22A(+1715+1732) 1mm_6 | 18 mer |
| 937 | GTGAAGTGGAACTGACCC | SHANK3 H22A(+1715+1732) 1mm_7 | 18 mer |
| 938 | GTGAAGCTGAACTGACCC | SHANK3 H22A(+1715+1732) 1mm_8 | 18 mer |
| 939 | GTGAAGCGTAACTGACCC | SHANK3 H22A(+1715+1732) 1mm_9 | 18 mer |
| 940 | GTGAAGCGGAACTAACCC | SHANK3 H22A(+1715+1732) mm1 | 18 mer |
| 941 | GGTGAAGCGGAACTGACC | SHANK3 H22A(+1716+1733) | 18 mer |
| 942 | TGTGAAGCGGAACTGACC | SHANK3 H22A(+1716+1733) 1mm_1 | 18 mer |
| 943 | GGTGAAGCGTAACTGACC | SHANK3 H22A(+1716+1733) 1mm_10 | 18 mer |
| 944 | GGTGAAGCGGAATTGACC | SHANK3 H22A(+1716+1733) 1mm_13 | 18 mer |
| 945 | GGTGAAGCGGAACTTACC | SHANK3 H22A(+1716+1733) 1mm_15 | 18 mer |
| 946 | GGTGAAGCGGAACTGATC | SHANK3 H22A(+1716+1733) 1mm_17 | 18 mer |
| 947 | GGTGAAGCGGAACTGACT | SHANK3 H22A(+1716+1733) 1mm_18 | 18 mer |
| 948 | GTTGAAGCGGAACTGACC | SHANK3 H22A(+1716+1733) 1mm_2 | 18 mer |
| 949 | GGTTAAGCGGAACTGACC | SHANK3 H22A(+1716+1733) 1mm_4 | 18 mer |
| 950 | GGTGAATCGGAACTGACC | SHANK3 H22A(+1716+1733) 1mm_7 | 18 mer |
| 951 | GGTGAAGTGGAACTGACC | SHANK3 H22A(+1716+1733) 1mm_8 | 18 mer |
| 952 | GGTGAAGCTGAACTGACC | SHANK3 H22A(+1716+1733) 1mm_9 | 18 mer |
| 953 | GGTGAAGCGGAACTAACC | SHANK3 H22A(+1716+1733) mm1 | 18 mer |
| 954 | AGGTGAAGCGGAACTGAC | SHANK3 H22A(+1717+1734) | 18 mer |
| 955 | AGGTGAAGCTGAACTGAC | SHANK3 H22A(+1717+1734) 1mm_10 | 18 mer |
| 956 | AGGTGAAGCGTAACTGAC | SHANK3 H22A(+1717+1734) 1mm_11 | 18 mer |
| 957 | AGGTGAAGCGGAATTGAC | SHANK3 H22A(+1717+1734) 1mm_14 | 18 mer |
| 958 | AGGTGAAGCGGAACTTAC | SHANK3 H22A(+1717+1734) 1mm_16 | 18 mer |
| 959 | AGGTGAAGCGGAACTGAT | SHANK3 H22A(+1717+1734) 1mm_18 | 18 mer |
| 960 | ATGTGAAGCGGAACTGAC | SHANK3 H22A(+1717+1734) 1mm_2 | 18 mer |
| 961 | AGTTGAAGCGGAACTGAC | SHANK3 H22A(+1717+1734) 1mm_3 | 18 mer |
| 962 | AGGTTAAGCGGAACTGAC | SHANK3 H22A(+1717+1734) 1mm_5 | 18 mer |
| 963 | AGGTGAATCGGAACTGAC | SHANK3 H22A(+1717+1734) 1mm_8 | 18 mer |
| 964 | AGGTGAAGTGGAACTGAC | SHANK3 H22A(+1717+1734) 1mm_9 | 18 mer |
| 965 | AGGTGAAGCGGAACTAAC | SHANK3 H22A(+1717+1734) mm1 | 18 mer |
| 966 | GAGGTGAAGCGGAACTGA | SHANK3 H22A(+1718+1735) | 18 mer |
| 967 | TAGGTGAAGCGGAACTGA | SHANK3 H22A(+1718+1735) 1mm_1 | 18 mer |
| 968 | GAGGTGAAGTGGAACTGA | SHANK3 H22A(+1718+1735) 1mm_10 | 18 mer |
| 969 | GAGGTGAAGCTGAACTGA | SHANK3 H22A(+1718+1735) 1mm_11 | 18 mer |
| 970 | GAGGTGAAGCGTAACTGA | SHANK3 H22A(+1718+1735) 1mm_12 | 18 mer |
| 971 | GAGGTGAAGCGGAATTGA | SHANK3 H22A(+1718+1735) 1mm_15 | 18 mer |
| 972 | GAGGTGAAGCGGAACTTA | SHANK3 H22A(+1718+1735) 1mm_17 | 18 mer |
| 973 | GATGTGAAGCGGAACTGA | SHANK3 H22A(+1718+1735) 1mm_3 | 18 mer |
| 974 | GAGTTGAAGCGGAACTGA | SHANK3 H22A(+1718+1735) 1mm_4 | 18 mer |
| 975 | GAGGTTAAGCGGAACTGA | SHANK3 H22A(+1718+1735) 1mm_6 | 18 mer |
| 976 | GAGGTGAATCGGAACTGA | SHANK3 H22A(+1718+1735) 1mm_9 | 18 mer |
| 977 | GAGGTGAAGCGGAACTAA | SHANK3 H22A(+1718+1735) mm1 | 18 mer |
| 978 | GGAGGTGAAGCGGAACTG | SHANK3 H22A(+1719+1736) | 18 mer |
| 979 | TGAGGTGAAGCGGAACTG | SHANK3 H22A(+1719+1736) 1mm_1 | 18 mer |
| 980 | GGAGGTGAATCGGAACTG | SHANK3 H22A(+1719+1736) 1mm_10 | 18 mer |
| 981 | GGAGGTGAAGTGGAACTG | SHANK3 H22A(+1719+1736) 1mm_11 | 18 mer |
| 982 | GGAGGTGAAGCTGAACTG | SHANK3 H22A(+1719+1736) 1mm_12 | 18 mer |
| 983 | GGAGGTGAAGCGTAACTG | SHANK3 H22A(+1719+1736) 1mm_13 | 18 mer |
| 984 | GGAGGTGAAGCGGAATTG | SHANK3 H22A(+1719+1736) 1mm_16 | 18 mer |
| 985 | GGAGGTGAAGCGGAACTT | SHANK3 H22A(+1719+1736) 1mm_18 | 18 mer |
| 986 | GTAGGTGAAGCGGAACTG | SHANK3 H22A(+1719+1736) 1mm_2 | 18 mer |
| 987 | GGATGTGAAGCGGAACTG | SHANK3 H22A(+1719+1736) 1mm_4 | 18 mer |
| 988 | GGAGTTGAAGCGGAACTG | SHANK3 H22A(+1719+1736) 1mm_5 | 18 mer |
| 989 | GGAGGTTAAGCGGAACTG | SHANK3 H22A(+1719+1736) 1mm_7 | 18 mer |
| 990 | GGAGGTGAAGCGGAACTA | SHANK3 H22A(+1719+1736) mm1 | 18 mer |
| 991 | AGGAGGTGAAGCGGAACT | SHANK3 H22A(+1720+1737) | 18 mer |
| 992 | AGGAGGTGAATCGGAACT | SHANK3 H22A(+1720+1737) 1mm_11 | 18 mer |
| 993 | AGGAGGTGAAGTGGAACT | SHANK3 H22A(+1720+1737) 1mm_12 | 18 mer |
| 994 | AGGAGGTGAAGCTGAACT | SHANK3 H22A(+1720+1737) 1mm_13 | 18 mer |
| 995 | AGGAGGTGAAGCGTAACT | SHANK3 H22A(+1720+1737) 1mm_14 | 18 mer |
| 996 | AGGAGGTGAAGCGGAATT | SHANK3 H22A(+1720+1737) 1mm_17 | 18 mer |
| 997 | ATGAGGTGAAGCGGAACT | SHANK3 H22A(+1720+1737) 1mm_2 | 18 mer |
| 998 | AGTAGGTGAAGCGGAACT | SHANK3 H22A(+1720+1737) 1mm_3 | 18 mer |
| 999 | AGGATGTGAAGCGGAACT | SHANK3 H22A(+1720+1737) 1mm_5 | 18 mer |
| 1000 | AGGAGTTGAAGCGGAACT | SHANK3 H22A(+1720+1737) 1mm_6 | 18 mer |
| 1001 | AGGAGGTTAAGCGGAACT | SHANK3 H22A(+1720+1737) 1mm_8 | 18 mer |
| 1002 | AGGAGGTGAAGCGAAACT | SHANK3 H22A(+1720+1737) mm1 | 18 mer |
| 1003 | AAGGAGGTGAAGCGGAAC | SHANK3 H22A(+1721+1738) | 18 mer |
| 1004 | AAGGAGGTGAATCGGAAC | SHANK3 H22A(+1721+1738) 1mm_12 | 18 mer |
| 1005 | AAGGAGGTGAAGTGGAAC | SHANK3 H22A(+1721+1738) 1mm_13 | 18 mer |
| 1006 | AAGGAGGTGAAGCTGAAC | SHANK3 H22A(+1721+1738) 1mm_14 | 18 mer |
| 1007 | AAGGAGGTGAAGCGTAAC | SHANK3 H22A(+1721+1738) 1mm_15 | 18 mer |
| 1008 | AAGGAGGTGAAGCGGAAT | SHANK3 H22A(+1721+1738) 1mm_18 | 18 mer |
| 1009 | AATGAGGTGAAGCGGAAC | SHANK3 H22A(+1721+1738) 1mm_3 | 18 mer |
| 1010 | AAGTAGGTGAAGCGGAAC | SHANK3 H22A(+1721+1738) 1mm_4 | 18 mer |
| 1011 | AAGGATGTGAAGCGGAAC | SHANK3 H22A(+1721+1738) 1mm_6 | 18 mer |
| 1012 | AAGGAGTTGAAGCGGAAC | SHANK3 H22A(+1721+1738) 1mm_7 | 18 mer |
| 1013 | AAGGAGGTTAAGCGGAAC | SHANK3 H22A(+1721+1738) 1mm_9 | 18 mer |
| 1014 | AAGGAGGTGAAGCGAAAC | SHANK3 H22A(+1721+1738) mm1 | 18 mer |
| 1015 | AAAGGAGGTGAAGCGGAA | SHANK3 H22A(+1722+1739) | 18 mer |
| 1016 | AAAGGAGGTTAAGCGGAA | SHANK3 H22A(+1722+1739) 1mm_10 | 18 mer |
| 1017 | AAAGGAGGTGAATCGGAA | SHANK3 H22A(+1722+1739) 1mm_13 | 18 mer |
| 1018 | AAAGGAGGTGAAGTGGAA | SHANK3 H22A(+1722+1739) 1mm_14 | 18 mer |
| 1019 | AAAGGAGGTGAAGCTGAA | SHANK3 H22A(+1722+1739) 1mm_15 | 18 mer |
| 1020 | AAAGGAGGTGAAGCGTAA | SHANK3 H22A(+1722+1739) 1mm_16 | 18 mer |
| 1021 | AAATGAGGTGAAGCGGAA | SHANK3 H22A(+1722+1739) 1mm_4 | 18 mer |
| 1022 | AAAGTAGGTGAAGCGGAA | SHANK3 H22A(+1722+1739) 1mm_5 | 18 mer |
| 1023 | AAAGGATGTGAAGCGGAA | SHANK3 H22A(+1722+1739) 1mm_7 | 18 mer |
| 1024 | AAAGGAGTTGAAGCGGAA | SHANK3 H22A(+1722+1739) 1mm_8 | 18 mer |
| 1025 | AAAGGAGGTGAAGCGAAA | SHANK3 H22A(+1722+1739) mm1 | 18 mer |
| 1026 | AAAAGGAGGTGAAGCGGA | SHANK3 H22A(+1723+1740) | 18 mer |
| 1027 | AAAAGGAGGTTAAGCGGA | SHANK3 H22A(+1723+1740) 1mm_11 | 18 mer |
| 1028 | AAAAGGAGGTGAATCGGA | SHANK3 H22A(+1723+1740) 1mm_14 | 18 mer |
| 1029 | AAAAGGAGGTGAAGTGGA | SHANK3 H22A(+1723+1740) 1mm_15 | 18 mer |
| 1030 | AAAAGGAGGTGAAGCTGA | SHANK3 H22A(+1723+1740) 1mm_16 | 18 mer |
| 1031 | AAAAGGAGGTGAAGCGTA | SHANK3 H22A(+1723+1740) 1mm_17 | 18 mer |
| 1032 | AAAATGAGGTGAAGCGGA | SHANK3 H22A(+1723+1740) 1mm_5 | 18 mer |
| 1033 | AAAAGTAGGTGAAGCGGA | SHANK3 H22A(+1723+1740) 1mm_6 | 18 mer |
| 1034 | AAAAGGATGTGAAGCGGA | SHANK3 H22A(+1723+1740) 1mm_8 | 18 mer |
| 1035 | AAAAGGAGTTGAAGCGGA | SHANK3 H22A(+1723+1740) 1mm_9 | 18 mer |
| 1036 | AAAAGGAGGTGAAGCGAA | SHANK3 H22A(+1723+1740) mm1 | 18 mer |
| 1037 | CAAAAGGAGGTGAAGCGG | SHANK3 H22A(+1724+1741) | 18 mer |
| 1038 | TAAAAGGAGGTGAAGCGG | SHANK3 H22A(+1724+1741) 1mm_1 | 18 mer |
| 1039 | CAAAAGGAGTTGAAGCGG | SHANK3 H22A(+1724+1741) 1mm_10 | 18 mer |
| 1040 | CAAAAGGAGGTTAAGCGG | SHANK3 H22A(+1724+1741) 1mm_12 | 18 mer |
| 1041 | CAAAAGGAGGTGAATCGG | SHANK3 H22A(+1724+1741) 1mm_15 | 18 mer |
| 1042 | CAAAAGGAGGTGAAGTGG | SHANK3 H22A(+1724+1741) 1mm_16 | 18 mer |
| 1043 | CAAAAGGAGGTGAAGCTG | SHANK3 H22A(+1724+1741) 1mm_17 | 18 mer |
| 1044 | CAAAAGGAGGTGAAGCGT | SHANK3 H22A(+1724+1741) 1mm_18 | 18 mer |
| 1045 | CAAAATGAGGTGAAGCGG | SHANK3 H22A(+1724+1741) 1mm_6 | 18 mer |
| 1046 | CAAAAGTAGGTGAAGCGG | SHANK3 H22A(+1724+1741) 1mm_7 | 18 mer |
| 1047 | CAAAAGGATGTGAAGCGG | SHANK3 H22A(+1724+1741) 1mm_9 | 18 mer |
| 1048 | CAAAAGGAGGTGAAGCGA | SHANK3 H22A(+1724+1741) mm1 | 18 mer |
| 1049 | ACAAAAGGAGGTGAAGCG | SHANK3 H22A(+1725+1742) | 18 mer |
| 1050 | ACAAAAGGATGTGAAGCG | SHANK3 H22A(+1725+1742) 1mm_10 | 18 mer |
| 1051 | ACAAAAGGAGTTGAAGCG | SHANK3 H22A(+1725+1742) 1mm_11 | 18 mer |
| 1052 | ACAAAAGGAGGTTAAGCG | SHANK3 H22A(+1725+1742) 1mm_13 | 18 mer |
| 1053 | ACAAAAGGAGGTGAATCG | SHANK3 H22A(+1725+1742) 1mm_16 | 18 mer |
| 1054 | ACAAAAGGAGGTGAAGTG | SHANK3 H22A(+1725+1742) 1mm_17 | 18 mer |
| 1055 | ACAAAAGGAGGTGAAGCT | SHANK3 H22A(+1725+1742) 1mm_18 | 18 mer |
| 1056 | ATAAAAGGAGGTGAAGCG | SHANK3 H22A(+1725+1742) 1mm_2 | 18 mer |
| 1057 | ACAAAATGAGGTGAAGCG | SHANK3 H22A(+1725+1742) 1mm_7 | 18 mer |
| 1058 | ACAAAAGTAGGTGAAGCG | SHANK3 H22A(+1725+1742) 1mm_8 | 18 mer |
| 1059 | ACAAAAGGAGGTGAAGCA | SHANK3 H22A(+1725+1742) mm1 | 18 mer |
| 1060 | TACAAAAGGAGGTGAAGC | SHANK3 H22A(+1726+1743) | 18 mer |
| 1061 | TACAAAAGGATGTGAAGC | SHANK3 H22A(+1726+1743) 1mm_11 | 18 mer |
| 1062 | TACAAAAGGAGTTGAAGC | SHANK3 H22A(+1726+1743) 1mm_12 | 18 mer |
| 1063 | TACAAAAGGAGGTTAAGC | SHANK3 H22A(+1726+1743) 1mm_14 | 18 mer |
| 1064 | TACAAAAGGAGGTGAATC | SHANK3 H22A(+1726+1743) 1mm_17 | 18 mer |
| 1065 | TACAAAAGGAGGTGAAGT | SHANK3 H22A(+1726+1743) 1mm_18 | 18 mer |
| 1066 | TATAAAAGGAGGTGAAGC | SHANK3 H22A(+1726+1743) 1mm_3 | 18 mer |
| 1067 | TACAAAATGAGGTGAAGC | SHANK3 H22A(+1726+1743) 1mm_8 | 18 mer |
| 1068 | TACAAAAGTAGGTGAAGC | SHANK3 H22A(+1726+1743) 1mm_9 | 18 mer |
| 1069 | TACAAAAGGAGGTGAAAC | SHANK3 H22A(+1726+1743) mm1 | 18 mer |
| 1070 | ATACAAAAGGAGGTGAAG | SHANK3 H22A(+1727+1744) | 18 mer |
| 1071 | ATACAAAAGTAGGTGAAG | SHANK3 H22A(+1727+1744) 1mm_10 | 18 mer |
| 1072 | ATACAAAAGGATGTGAAG | SHANK3 H22A(+1727+1744) 1mm_12 | 18 mer |
| 1073 | ATACAAAAGGAGTTGAAG | SHANK3 H22A(+1727+1744) 1mm_13 | 18 mer |
| 1074 | ATACAAAAGGAGGTTAAG | SHANK3 H22A(+1727+1744) 1mm_15 | 18 mer |
| 1075 | ATACAAAAGGAGGTGAAT | SHANK3 H22A(+1727+1744) 1mm_18 | 18 mer |
| 1076 | ATATAAAAGGAGGTGAAG | SHANK3 H22A(+1727+1744) 1mm_4 | 18 mer |
| 1077 | ATACAAAATGAGGTGAAG | SHANK3 H22A(+1727+1744) 1mm_9 | 18 mer |
| 1078 | ATACAAAAGGAGGTGAAA | SHANK3 H22A(+1727+1744) mm1 | 18 mer |

TABLE 2
Exemplary length-matured ASOs targeting a region
(SEQ ID NO: 1079) of the 3′ UTR of SHANK3

| SEQ ID | | | ASO |
|---|---|---|---|
| NO. | ASO Sequence | ASO Name | length |
| 1080 | ACTGACCCTGTAAAGTTAAA | SHANK3_H22A(+1703+1722) | 20 mer |
| 1081 | GAACTGACCCTGTAAAGTTA | SHANK3_H22A(+1705+1724) | 20 mer |
| 1082 | CGGAACTGACCCTGTAAAGT | SHANK3_H22A(+1707+1726) | 20 mer |
| 1083 | ACTGACCCTGTAAAGT | SHANK3_H22A(+1707+1722) | 16 mer |
| 1084 | AACTGACCCTGTAAA | SHANK3_H22A(+1709+1723) | 15 mer |
| 1085 | ACCCTGTAAAGTTAAAAA | SHANK3_H22A(+1701+1718) | 18 mer |
| 1086 | TGACCCTGTAAAGTTAAAAA | SHANK3_H22A(+1701+1720) | 20 mer |
| 1087 | AGCGGAACTGACCCTGTAAA | SHANK3_H22A(+1709+1728) | 20 mer |
| 1088 | AAGCGGAACTGACCCTGTAA | SHANK3_H22A(+1710+1729) | 20 mer |
| 1089 | CATACAAAAGGAGGTGAA | SHANK3_H22A(+1728+1745) | 18 mer |
| 1090 | TCCATACAAAAGGAGGTGAA | SHANK3_H22A(+1728+1747) | 20 mer |
| 1091 | ATGGAACTCCATACAAAA | SHANK3_H22A(+1737+1754) | 18 mer |
| 1092 | AATTGAACGGAACCAAAA | SHANK3_H22A(+1679+1696) | 18 mer |
| 1093 | TAAAGTTAAAAACCCAAA | SHANK3_H22A(+1695+1712) | 18 mer |
| 1094 | GCGGAACTGACCCTGTA | SHANK3_H22A(+1711+1727) | 17 mer |
| 1095 | CGGAACTGACCCTGTAAAGTTAAAA | SHANK3_H22A(+1702+1726) | 25 mer |

TABLE 3
Exemplary chemistry-matured ASOs a region
(SEQ ID NO: 1079) of the 3′ UTR of SHANK3

| SEQ | | | |
|---|---|---|---|
| ID | | | ASO |
| NO. | ASO Sequence | ASO Name | length |
| 1096 | AACT*GACC*CTGT*AAA | SHANK3_H22A(+1709+1723)_31na_4_8_12 | 15 mer |
| 1097 | AACT*GAC*CCTGT*AAA | SHANK3_H22A(+1709+1723)_31na_4_7_12 | 15 mer |
| 1098 | AACT*GACCCTGT*AAA | SHANK3_H22A(+1709+1723)_21na_4_12 | 15 mer |
| 1099 | AACTGAC*CCTGT*AAA | SHANK3_H22A(+1709+1723)_21na_7_12 | 15 mer |
| 1100 | AACTGA*CCCTGT*AAA | SHANK3_H22A(+1709+1723)_21na_6_12 | 15 mer |
| 1101 | A*ACTGACCCT*GTAAA | SHANK3_H22A(+1709+1723)_21na_1_10 | 15 mer |
| 1102 | AACTGA*CCCTGTAAA* | SHANK3_H22A(+1709+1723)_21na_6_15 | 15 mer |
| 1103 | A*ACTGACC*CTGTAAA* | SHANK3_H22A(+1709+1723)_31na_1_8_15 | 15 mer |
| 1104 | AGCG*SA*ACTGACCCTGTAAA | SHANK3_H22A(+1709+1728)_1spr_5_21na_4_6 | 20 mer |
| 1105 | AGCG*SA*ACTGACSCTGTAAA | SHANK3_H22A(+1709+1728)_2spr_5_13_21na_4_6 | 20 mer |
| 1106 | AGCGGAACT*SA*CCCTGTAAA | SHANK3_H22A(+1709+1728)_1spr_10_21na_9_11 | 20 mer |
| 1107 | AGC*GGAACTGACCCTGT*AAA | SHANK3_H22A(+1709+1728)_21na_3_17 | 20 mer |
| 1108 | AGCG*SAACTGACCCTGT*AAA | SHANK3_H22A(+1709+1728)_1spr_5_21na_4_17 | 20 mer |
| 1109 | GCG*SA*ACTGACCCTGTA | SHANK3_H22A(+1711+1727)_1spr_4_21na_3_5 | 17 mer |
| 1110 | GCGGAACT*SA*CCCTGTA | SHANK3_H22A(+1711+1727)_1spr_9_2lna_8_10 | 17 mer |
| 1111 | GCGGA*ACTGA*CCCT*GTA | SHANK3_H22A(+1711+1727)_31na_5_10_14 | 17 mer |
| 1112 | GC*GGAACTGACCCTGT*A | SHANK3_H22A(+1711+1727)_21na_2_16 | 17 mer |
| 1113 | GC*SGAACTGACCCTGT*A | SHANK3_H22A(+1711+1727)_1spr_3_2lna_2_16 | 17 mer |
| 1114 | ATG*SA*ACTCCATACAAAA | SHANK3_H22A(+1737+1754)_1spr_4_21na_3_5 | 18 mer |
| 1115 | ATG*SA*ACTCCATASAAAA | SHANK3_H22A(+1737+1754)_2spr_4_14_21na_3_5 | 18 mer |
| 1116 | ATG*SA*ACTCSATACAAAA | SHANK3_H22A(+1737+1754)_2spr_4_10_21na_3_5 | 18 mer |
| 1117 | ATGGAACTC*SA*TACAAAA | SHANK3_H22A(+1737+1754)_1spr_10_21na_9_11 | 18 mer |
| 1118 | ATGGAACTCCATA*SA*AAA | SHANK3_H22A(+1737+1754)_1spr_14_21na_13_15 | 18 mer |
| 1119 | AT*GGAACTCCATACAA*AA | SHANK3_H22A(+1737+1754)_21na_2_16 | 18 mer |
| 1120 | ATG*GAACTCCATACAA*AA | SHANK3_H22A(+1737+1754)_21na_3_16 | 18 mer |
| 1121 | TAAAGTTAAAAAC*SC*AAA | SHANK3_H22A(+1695+1712)_1spr_14_21na_13_15 | 18 mer |
| 1122 | TAA*SG*TTAAAAACCCAAA | SHANK3_H22A(+1695+1712)_1spr_4_21na_3_5 | 18 mer |
| 1123 | TAA*SG*TTAAAAACSCAAA | SHANK3_H22A(+1695+1712)_2spr_4_14_21na_3_5 | 18 mer |
| 1124 | TAAAGTTAA*SA*ACCCAAA | SHANK3_H22A(+1695+1712)_1spr_10_21na_9_11 | 18 mer |
| 1125 | TAA*AGTT*AAAAACCCA*AA | SHANK3_H22A(+1695+1712)_31na_3_7_16 | 18 mer |
| 1126 | AATTGAA*SG*GAACCAAAA | SHANK3_H22A(+1679+1696)_1spr_8_21na_7_9 | 18 mer |
| 1127 | AATTGAACG*SA*ACCAAAA | SHANK3_H22A(+1679+1696)_1spr_10_2lna_9_11 | 18 mer |
| 1128 | AATTGAACGGAA*SC*AAAA | SHANK3_H22A(+1679+1696)_1spr_13_21na_12_14 | 18 mer |
| 1129 | AATTG*SA*CGGAACCAAAA | SHANK3_H22A(+1679+1696)_1spr_6_21na_5_7 | 18 mer |
| 1130 | AATTSAACG*SA*ACCAAAA | SHANK3_H22A(+1679+1696)_2spr_5_10_21na_9_11 | 18 mer |
| 1131 | AATTG*SA*CGGAASCAAAA | SHANK3_H22A(+1679+1696)_2spr_6 13_2lna_5_7 | 18 mer |
| 1132 | AA*TTGAACGGA*ACCAAAA | SHANK3_H22A(+1679+1696)_21na_2_11 | 18 mer |
| 1133 | AA*TTGAACGSA*ACCAAAA | SHANK3_H22A(+1679+1696)_1spr_10_2lna_2_11 | 18 mer |

(Note:
LNA modified nucleotides are denoted by a ″*″ and abasic spacers with an ″S″.)

1-64. (canceled)

65. An antisense oligonucleotide (ASO), wherein the nucleotide sequence of the antisense oligonucleotide consists of SEQ ID NO:1092, and wherein each nucleotide of the ASO comprises a 2′-O-methoxyethyl moiety.

66. A pharmaceutical composition comprising the ASO according to claim 65 and a pharmaceutically acceptable carrier.

67. A method for treating a condition associated with SHANK3 haploinsufficiency, the method comprising administering to a subject in need thereof a therapeutically effective amount of the ASO according to claim 65.

68. The method according to claim 67, wherein the condition is Phelan-McDermid syndrome, an autism spectrum disorder, schizophrenia, or an intellectual disability.

69. The method according to claim 67, wherein the condition is Phelan-McDermid syndrome.

70. A method for treating a condition associated with SHANK3 haploinsufficiency, the method comprising administering to a subject in need thereof a therapeutically effective amount of the pharmaceutical composition according to claim 66.

71. The method according to claim 70, wherein the condition is Phelan-McDermid syndrome, an autism spectrum disorder, schizophrenia, or an intellectual disability.

72. The method according to claim 70, wherein the condition is Phelan-McDermid syndrome.
